(19) United States
(12) Patent Application Publication
(10) Pub. No.: US 2023/0193285 A1
(43) Pub. Date: Jun. 22, 2023

(54) BISPECIFIC PERSONALIZED APTAMERS

(71) Applicant: Aummune Ltd., Tel Aviv-Yafo (IL)

(72) Inventors: Guy NEEV, Bnei Dror (IL); Irit Carmi-Levy, Tel Aviv (IL); Yaniv Amir, Tel Aviv (IL); Erez Lavi, Bat Yam (IL); Nir Skalka, Rehovot (IL); Neta Zilony-Hanin, Tel Aviv (IL); Zohar Pode, Tel Aviv (IL)

(21) Appl. No.: 17/926,067

(22) PCT Filed: May 19, 2021
(86) PCT No.: PCT/IB2021/000340

§ 371 Date: Nov. 17, 2022

Related U.S. Application Data
(60) Provisional application No. 63/121,079, filed on Dec. 3, 2020.
(60) Provisional application No. 63/027,629, filed on May 20, 2020.

(51) Int. Cl. C12N 15/115; A61P 35/00; A61K 45/06; A61K 31/7088

(52) U.S. Cl. CPC C12N 15/115; A61K 31/7088; A61K 45/06; A61P 35/00; C12N 2310/16; C12N 2310/17; C12N 2310/51; C12N 2310/315; C12N 2310/321; C12N 2310/322; C12N 2310/335; C12N 2310/351; C12N 2310/3231; C12N 2310/3515; C12N 2310/3519; C12N 2330/31

(57) ABSTRACT

Provided herein are bispecific personalized aptamers that induce the cell death of cancer cells and methods of use thereof.

### RELATED APPLICATIONS

This application claims the benefit of priority to U.S. Provisional Pat. Application serial numbers 63/027,629, filed May 20, 2020, and 63/121,079, filed Dec. 3, 2020, each of which is hereby incorporated by reference in its entirety.

### BACKGROUND

Aptamers are short, single-stranded nucleic acid oligomers that can bind to a specific target molecule. Aptamers are typically selected from a large random pool of oligonucleotides in an iterative process.

Aptamer-based therapeutics offer a number of advantages over traditional antibody-based therapeutics, including their quick chemical production, their amenability to chemical modification, their high stability and their lack of immunogenicity. Thus, aptamers that are capable of selectively targeting and killing cancer cells would have great potential as anti-cancer therapeutics.

### SUMMARY

Provided herein are bispecific personalized aptamers useful as cancer therapeutics, as well as pharmaceutical compositions comprising such bispecific personalized aptamers, and methods of making and using such aptamers. In certain embodiments, the bispecific personalized aptamers provided herein are cancer therapeutic species comprised of three functionally distinct moieties: (1) a cancer-cell target-specific moiety able to bind and induce cytotoxicity on the target cancer cell; (2) an immune-cell engaging moiety; (3) and a CpG motif.

In certain aspects, the compositions and methods disclosed herein provide and facilitate patient-tailored cancer therapeutics to treat patients with individualized solutions optimized for the unique set of conditions and potential drug targets presented by each patient as reflected by fresh sample tissue of their tumor. In certain embodiments, the bispecific personalized aptamers disclosed herein are composed of two arms. One aptameric arm is directed against an individual subject’s tumor. This tumor-targeting arm is a functional aptamer selected for its ability to both bind target cancer cells as well as specifically induce cell death on those tumor cells. This moiety is variable and custom-made for each individual patient. The second aptameric arm targets immune effector cells, functioning as an “engager” and leading to tumor cell lysis by the immune cells. This latter immune-modulating arm is designed to be shared across different patients. In embodiments, the two aptamer arms of the bispecific structure are bridged together by nucleic-base hybridization of single stranded overhangs of complementary sequences. This hybridization domain is CpG-rich and designed to induce toll-like receptor 9 (TLR9)-mediated Antigen Presenting Cells (APC) stimulation and increase uptake of tumor antigens. Thus, in certain embodiments, the disclosed aptamers’ bispecificity coupled with their TLR9 agonistic activity makes them valuable components in a multi-faceted approach to treating cancers.

In certain aspects, provided herein are bispecific personalized aptamers that comprise a cancer cell-binding strand that selectively binds to and/or selectively kills cancer cells (e.g., breast cancer cells, colorectal carcinoma cells), including by inducing apoptosis. The bispecific personalized aptamers also comprise an immune effector cell-binding strand that, for example, facilitates cancer cell lysis through T cell or natural killer (NK) cell-mediated cytotoxicity. In some embodiments, the cancer cell-binding strand is linked to the immune effector cell-binding strand by a CpG-rich TLR9 agonistic sequence that induces TLR9-mediated APCs stimulation and/or increased uptake of tumor antigens. In some aspects, provided herein are pharmaceutical compositions comprising such bispecific personalized aptamers, methods of using such bispecific personalized aptamers to treat cancer and/or to kill cancer cells and methods of making such bispecific personalized aptamers.

In certain aspects, provided herein are bispecific personalized aptamers comprising (a) a cancer cell-binding strand that specifically binds to a target expressed on a cancer cell; (b) a TLR9 agonistic CpG motif; and (c) an immune effector cell-binding strand that specifically binds to an immune effector cell, wherein the cancer cell-binding strand is linked to the immune effector cell-binding strand by the CpG motif.

In some embodiments, the cancer cell-binding strand induces cell death (e.g., apoptosis) when contacted to a cancer cell. In some embodiments, the cancer cell is a patient-derived cancer cell. The cancer cell may be a solid tumor cell (e.g., a breast cancer cell or a colorectal carcinoma cell), a sarcoma cell (e.g., a soft tissue sarcoma cell), or a hematological cancer cell (e.g., a lymphoma cell). The cancer cell-binding strand induces cell death when contacted to the cancer cell in vitro or in vivo. In some embodiments, the immune effector cell-binding strand mediates lysis of the cancer cell through T cell or NK cell-mediated cytotoxicity. In some embodiments, the cancer cell-binding strand and the immune effector cell-binding strand are linked together by hybridization of a 5′ sequence of the cancer cell-binding strand to a 5′ sequence of the immune effector cell-binding strand. In some embodiments, the 5′ sequence of the cancer cell-binding strand hybridizes to the 5′ sequence of the immune effector cell-binding strand to form the TLR9 agonist sequence. In some embodiments, the TLR9 agonist sequence comprises a double-stranded region of a CpG motif. In some embodiments, the CpG motif induces TLR9-mediated APCs stimulation and/or increased uptake of tumor antigens. In some embodiments, the TLR9 agonist sequence induces an anti-tumor immune response. In some embodiments, the TLR9 agonist sequence induces IFNα secretion, IL6 secretion, and/or B-cell activation.

In some embodiments, the CpG motif is a double-stranded nucleic acid sequence comprising a sequence that is at least 60% identical (e.g., at least 65% identical, at least 70% identical, at least 75% identical, at least 80% identical, at least 85% identical, at least 90% identical, at least 92% identical, at least 94% identical, at least 96% identical, at least 98% identical) to any one of SEQ ID NOs: 63-66. In some embodiments, the CpG motif is a double-stranded nucleic acid sequence comprising a sequence of any one of SEQ ID NOs: 63-66.

In certain embodiments, the CpG motif is a double-stranded nucleic acid sequence comprising at least 12 (e.g., at least 13, at least 14, at least 15, at least 16, at least 17, at least 18, at least 19, at least 20, at least 21, at least 22) consecutive nucleotides of any one of SEQ ID NO: 63-66. In some embodiments, the CpG motif provided herein has a sequence consisting essentially of SEQ ID NOs: 63-66. In certain embodiments, the CpG motif provided herein has a sequence consisting of SEQ ID NO: 63-66.

In certain embodiments, the CpG motif is no more than 35 nucleotides in length (e.g., no more than 34 nucleotides in length, no more than 33 nucleotides in length, no more than 32 nucleotides in length, no more than 31 nucleotides in length, no more than 30 nucleotides in length, no more than 29 nucleotides in length, no more than 28 nucleotides in length, no more than 27 nucleotides in length, no more than 26 nucleotides in length, no more than 25 nucleotides in length, no more than 24 nucleotides in length, no more than 23 nucleotides in length, or no more than 22 nucleotides in length).

In certain embodiments, the cancer cell-binding strand is a personalized aptamer strand selected to binding and/or killing tumor cells obtained from an individual patient (e.g., selected using aptamer selection methods provided herein). In some embodiments, the cancer cell-binding strand binds to a cancer antigen. In certain embodiments the cancer antigen is selected from Major histocompatibility complex (MHC)- tumor-associated antigens (TAA) peptide complexes, Prostate Membrane Antigen (PSMA), Cancer antigen 15-3 (CA-15-3), Carcinoembryonic antigen (CEA), Cancer antigen 125 (CA-125), Tyrosinase, glycoprotein 100 (gp100), Melanoma Antigen Recognized by T-cells 1 (MART-1)/melan-A, heat shock protein 70 (HSP70)-2-m, human leukocyte antigen (HLA)-A2-R17OJ, human papillomavirus 16 (HPV16)-E7, Mucin 1 (MUC-1), human epidermal growth factor receptor 2 (HER-2)/neu, or Mammaglobin-A. In some embodiments, the cancer cell-binding strand comprises a nucleic acid sequence that is at least 60% identical (e.g., at least 65% identical, at least 70% identical, at least 75% identical, at least 80% identical, at least 85% identical, at least 90% identical, at least 92% identical, at least 94% identical, at least 96% identical, at least 98% identical) to any one of SEQ ID NOs: 43-62 or 107-115. In some embodiments, the cancer cell-binding strand comprises a nucleic acid sequence of any one of SEQ ID NOs: 43-62 or 107-115.

In certain embodiments, the cancer cell-binding strand comprises at least 30 (e.g., at least 35, at least 40, at least 45, at least 50, at least 55, at least 60) consecutive nucleotides of any one of SEQ ID NO: 43-62 or 107-115. In some embodiments, the cancer cell-binding strand provided herein has a sequence consisting essentially of SEQ ID NOs: 43-62 or 107-115. In certain embodiments, the cancer cell-binding strand provided herein has a sequence consisting of SEQ ID NO: 43-62 or 107-115.

In certain embodiments, the cancer cell-binding strand is no more than 120 nucleotides in length (e.g., no more than 115 nucleotides in length, no more than 110 nucleotides in length, no more than 105 nucleotides in length, no more than 100 nucleotides in length, no more than 95 nucleotides in length, no more than 90 nucleotides in length, no more than 85 nucleotides in length, no more than 80 nucleotides in length, no more than 75 nucleotides in length, no more than 70 nucleotides in length, no more than 69 nucleotides in length, no more than 68 nucleotides in length, no more than 67 nucleotides in length, no more than 66 nucleotides in length, no more than 65 nucleotides in length, no more than 64 nucleotides in length, or no more than 63 nucleotides in length). In certain embodiment, the cancer cell-binding strand is about 63 nucleotides in length.

In some embodiments, the cancer cell-binding strands are 53-73 nucleotides in length. In certain embodiments, the cancer cell-binding strands are 58-68 nucleotides in length. In certain embodiments, the cancer cell-binding strands are about 63 nucleotides in length. In some embodiments the cancer cell-binding strands comprise a cancer-targeting moiety of about 40 nucleotides in length. In certain embodiments, the cancer cell-binding strands comprise a CpG complementary motif of about 23 nucleotides.

In some embodiments, the immune effector cell-binding strand binds to an antigen expressed by T cells (e.g., CD8+ T cell), NK cells, B cells, macrophages, dendritic cells, neutrophils, basophils or eosinophils. In some embodiments, the immune effector cell-binding strand binds to an immune effector cell antigen selected from CD16, Notch-2, other Notch family members, KCNK17, CD3, CD28, 4-1BB, CTLA-4, ICOS, CD40L, PD-1, OX40, LFA-1, CD27 PARP16, IGSF9, SLC15A3, WRB and GALR2.

In some embodiments, the immune effector cell-binding strand comprises a nucleic acid sequence that is at least 60% identical (e.g., at least 65% identical, at least 70% identical, at least 75% identical, at least 80% identical, at least 85% identical, at least 90% identical, at least 92% identical, at least 94% identical, at least 96% identical, at least 98% identical) to any one of SEQ ID NOs: 1-42, 88-106 or 116. In some embodiments, the immune effector cell-binding strand comprises a nucleic acid sequence of any one of SEQ ID NOs: 1-42, 88-106 or 116.

In certain embodiments, the immune effector cell-binding strand comprises at least 20 (e.g., at least 25, at least 30, at least 35, at least 40, at least 45, at least 50) consecutive nucleotides of any one of SEQ ID NOs: 1-42, 88-106 or 116. In some embodiments, the immune effector cell-binding strand provided herein has a sequence consisting essentially of SEQ ID NOs: 1-42, 88-106 or 116. In certain embodiments, the immune effector cell-binding strand provided herein has a sequence consisting of SEQ ID NO: 1-42, 88-106 or 116.

In certain embodiments, the immune effector cell-binding strand is no more than 120 nucleotides in length (e.g., no more than 115 nucleotides in length, no more than 110 nucleotides in length, no more than 105 nucleotides in length, no more than 100 nucleotides in length, no more than 95 nucleotides in length, no more than 90 nucleotides in length, no more than 85 nucleotides in length, no more than 80 nucleotides in length, no more than 75 nucleotides in length, no more than 74 nucleotides in length, or no more than 73 nucleotides in length). In certain embodiments, the immune effector cell-binding strand is about 73 nucleotides in length.

In some embodiments, the immune effector cell-binding strands are 63-83 nucleotides in length. In certain embodiments, the immune effector cell-binding strands are 68-78 nucleotides in length. In certain embodiments, the immune effector cell-binding strands are about 73 nucleotides in length. In some embodiments the immune effector cell-binding strands comprise a cancer-targeting moiety of about 50 nucleotides in length. In certain embodiments, the immune effector cell-binding strands comprise a CpG complementary motif of about 23 nucleotides.

In some embodiments, the bispecific personalized aptamer comprises a combination of two strands, with one strand selected from any one of SEQ ID NOs: 1-42, 88-106 or 116, and the other strand selected from any one of SEQ ID NOs: 43-62 or 107-115. For example, in certain embodiments, the paired strands are selected from SEQ ID NOs: 29 and 54, 29 and 50, 32 and 50, 33 and 48, 41 and 49, 34 and 59.

In some embodiments, the bispecific personalized aptamers provided herein comprise one or more chemical modifications. In some embodiments, the bispecific personalized aptamers are chemically modified with poly-ethylene glycol (PEG) (e.g., attached to the 5′ end or the 3′ end of the aptamer). In some embodiments, the bispecific personalized aptamers comprise a 5′ end cap. In certain embodiments, the aptamers comprise a 3′ end cap (e.g., is an inverted thymidine, biotin). In some embodiments, the bispecific personalized aptamers comprise one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 30 31, 32, 33, 34, 35, 36, 37, 38, 39, 40, 41, 42, 43, 44, 45, 46, 47, 48, 49, 50, 51, 52, 53, or 54) 2′ sugar substitutions (e.g. a 2′-fluoro, a 2′-amino, or a 2′-O-methyl substitution). In certain embodiments, the bispecific personalized aptamers comprise locked nucleic acid (LNA), unlocked nucleic acid (UNA) and/or 2′deozy-2′fluoro-D-arabinonucleic acid (2′-F ANA) sugars in their backbone.

In certain embodiments, the aptamers comprise one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 30 31, 32, 33, 34, 35, 36, 37, 38, 39, 40, 41, 42, 43, 44, 45, 46, 47, 48, 49, 50, 51, 52, 53, or 54) methylphosphonate internucleotide bonds and/or a phosphorothioate (PS) internucleotide bonds. In certain embodiments, the double-stranded CpG motif comprises a partial PS modification. In certain embodiments, 5 nucleotides from 5′ ends of the double-stranded CpG motif are modified. In other embodiments, 5 nucleotides from both 5′ and 3′ ends of the double-stranded CpG motif are modified. In certain embodiments, the double-stranded CpG motif comprises a complete PS modification. In certain embodiments, the bispecific personalized aptamers comprise one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 30 31, 32, 33, 34, 35, 36, 37, 38, 39, 40, 41, 42, 43, 44, 45, 46, 47, 48, 49, 50, 51, 52, 53, or 54) triazole internucleotide bonds. In certain embodiments, the bispecific personalized aptamers are modified with a cholesterol or a dialkyl lipid (e.g., on their 5′ end). In some embodiments, the bispecific personalized aptamers comprise one or more modified bases.

In certain embodiments, the bispecific personalized aptamers provided herein are DNA aptamers (e.g., D-DNA aptamers or enantiomer L-DNA aptamers). In some embodiments, the bispecific personalized aptamers provided herein are RNA aptamers (e.g., D-RNA aptamers or enantiomer L-RNA aptamers). In some embodiments, the bispecific personalized aptamers comprise a mixture of DNA and RNA.

In certain aspects, provided herein are pharmaceutical compositions comprising a bispecific personalized aptamer (e.g., a therapeutically effective amount of a bispecific personalized aptamer) provided herein. In some embodiments, the pharmaceutical compositions further comprising a pharmaceutically acceptable carrier. In some embodiments, the pharmaceutical composition is formulated for parenteral administration.

In certain embodiments, the pharmaceutical composition is for use in treating cancer. In some embodiments, the cancer is a solid tumor (e.g., a breast cancer). In certain embodiments, the cancer is a carcinoma (e.g., a colorectal carcinoma).

In certain aspects, provided herein is a method of treating cancer in a subject, the method comprising administering to the subject a bispecific personalized aptamer (e.g., a therapeutically effective amount of a bispecific personalized aptamer) and/or a pharmaceutical composition provided herein. In some embodiments, the administration is parenteral administration (e.g., subcutaneous administration). The administration may be an intratumoral injection or a peritumoral injection. In some embodiments, two or more doses are administered. In certain embodiments, at least 10 to 12 doses are administered. In some embodiments, the administration to the subject of the two or more doses are separated by at least 1 day.

In some embodiments, the cancer is a solid tumor (e.g., a breast cancer, head and neck squamous cell carcinoma, adenoid cystic carcinoma, bladder cancer, pancreatic cancer, hepatocellular carcinoma, melanoma, merkel cell carcinoma, or a colorectal carcinoma). In some embodiment, the solid tumor is accessible for intratumoral administration. In certain embodiment, the cancer is a sarcoma (e.g., soft tissue sarcoma). In certain embodiments, the cancer is a hematologic cancer (e.g., a lymphoma). In certain embodiments, the subject is a subject who has received chemotherapy.

In some embodiments, the therapeutic methods provided herein further comprise administering to the subject an additional cancer therapy. In some embodiments, the additional cancer therapy comprises chemotherapy. In certain embodiments, the additional cancer therapy comprises radiation therapy. In some embodiments, the additional cancer therapy comprises surgical removal of a tumor. In certain embodiments, the additional cancer therapy comprises administration of an immune checkpoint inhibitor (e.g., an anti-PD-1 antibody, an anti-PD-L1 antibody, an anti-PD-L2 antibody, or an anti-CTLA4 antibody) to the subject.

In certain aspects, provided herein is a method of killing a cancer cell, the method comprising contacting the cancer cell with a bispecific personalized aptamer provided herein. In some embodiments, the cancer cell is killed by apoptosis, necrosis, immunological cell death (ICD), autophagy or necroptosis. In some embodiments, the cancer cell is a solid tumor cell (e.g., a breast cancer cell or a colorectal carcinoma cell), a sarcoma cell (e.g., a soft tissue sarcoma cell), or a hematologic cancer cell (e.g., a lymphoma cell). In some embodiments, the cancer cell is killed when contacted with the bispecific personalized aptamer in vitro. In certain embodiments, the cancer cell is killed when contacted with the bispecific personalized aptamer in vivo (e.g., in a human and/or an animal model).

In certain aspects, provided herein is a method of making a bispecific personalized aptamer. In some embodiments, the method comprises (1) synthesizing a cancer cell-binding strand; (2) synthesizing an immune effector cell-binding strand; (3) hybridizing both strands to form the bispecific personalized aptamer.

In some embodiments, the cancer cell-binding strand is identified using a systematic evolution of ligands by exponential enrichment (selex) process. In certain embodiments, multiple rounds (e.g., 3 rounds) of binding selex is performed using targeted cancer cells to identify aptamers than bind to the cancer cell target. In certain embodiments, a functional selex assay is also performed via a process comprising: (a) contacting cancer cells with a plurality of particles on which are immobilized a library of aptamer clusters (“aptamer cluster particles”), wherein at least a subset of the immobilized aptamer clusters bind to at least a subset of the cancer cells to form cell-aptamer cluster particle complexes; (b) incubating the cell-aptamer cluster particle complexes for a period of time sufficient for at least some of the cancer cells in the cell-aptamer cluster particle complexes to undergo cell function; (c) detecting the cell-aptamer cluster particle complexes undergoing the cell function (e.g., using a functional reporter added to the reaction either before or after the aptamer cluster particle complexes are formed); (d) separating cell-aptamer cluster particle complexes comprising cancer cell undergoing the cell function detected in step (c) from other cell-aptamer cluster particle complexes; (e) amplifying the aptamers in the separated cell-aptamer cluster particle complexes to generate a functionally enriched population of aptamers; and (f) identifying the enriched population of aptamers via sequencing, thereby identifying the cancer cell-binding strand.

In some embodiments, steps (c) and (d) are performed using a flow cytometer. In some embodiments, the methods described herein further comprise separating the aptamer cluster particles from the target cells in the cell-aptamer cluster particle complexes separated in step (d). In some embodiments, the methods described herein further comprise the step of dissociating the aptamers from the particles in the separated aptamer cluster particles. In some embodiments, the methods described herein further comprise a step (e′) after step (e) and before step (f): (i) forming aptamer cluster particles from the functionally enriched population of aptamers of step (e); and (ii) repeating steps (a) - (e) using the newly formed aptamer cluster particles to generate a further functionally enriched population of aptamers. In some embodiments, step (e′) is repeated at least 2 (e.g., at least 3, at least 4, at least 5, at least 6, at least 7, at least 8, at least 9, at least 10) times. In some embodiments, step (e′) further comprises applying a restrictive condition in the successive rounds of enrichment. In some embodiments, the restrictive condition is selected from: (i) reducing the total number of particles, (ii) reducing copy number of aptamers per particle, (iii) reducing the total number of target cells, (iv) reducing the incubation period, and (v) introducing errors to the aptamer sequences by amplifying the population of aptamers using error-prone polymerase. In some embodiments, the further enriched population of aptamers of step (e′) has decreased sequence diversity compared to the library of aptamer clusters of step (a) by, for example, a factor of at least 1.1, 1.2, 1.3, 1.4, 1.5, 1.6, 1.7, 1.8, 1.9, 2.0, 2.1, 2.2, 2.3, 2.4, or 2.5. In some embodiments, each round of step (e′) enriches the population of aptamers for aptamers that modulate the cellular function by, for example, a factor of at least 1.1, 1.2, 1.3, 1.4, 1.5, 1.6, 1.7, 1.8, 1.9, 2.0, 2.1, 2.2, 2.3, 2.4, or 2.5. In some embodiments, the period of time is from about 10 minutes to about 5 days (e.g., from about 1.5 hours to about 72 hours, or from about 1.5 hours to about 24 hours).

In some embodiments, the cancer cell is incubated with a reporter of the cell function prior to, during, or after contacting the cancer cell with the aptamer cluster particles. In some embodiments, the cancer cell is contacted with the reporter of the cell function prior to, during, or after step (b). In some embodiments, the reporter of the cell function is a fluorescent dye. In some embodiments, the cell function is cell viability, cell death (e.g., apoptosis, non-programmed cell death), or cell proliferation. In some embodiments, the methods described herein further comprises the step of isolating the cancer cell from a patient prior to step (a). In some embodiments, the cancer cell is isolated from a tumor biopsy or resection.

In some embodiments, the method comprises synthesizing (e.g., chemically synthesizing) a cancer cell-binding strand comprising a nucleic acid sequence that is at least 60% identical (e.g., at least 65% identical, at least 70% identical, at least 75% identical, at least 80% identical, at least 85% identical, at least 90% identical, at least 92% identical, at least 94% identical, at least 96% identical, at least 98% identical) to any one of SEQ ID NOs: 43-62 or 107-115. In some embodiments, the method comprises synthesizing a cancer cell-binding strand comprising a nucleic acid sequence of any one of SEQ ID NOs: 43-62 or 107-115. In certain embodiments, the method comprises synthesizing a cancer cell-binding strand comprising a nucleic acid sequence that comprises at least 30 (e.g., at least 35, at least 40, at least 45, at least 50, at least 55, at least 60) consecutive nucleotides of any one of SEQ ID NO: 43-62 or 107-115. In some embodiments, the method comprises synthesizing a cancer cell-binding strand having a sequence consisting essentially of SEQ ID NOs: 43-62 or 107-115. In certain embodiments, the method comprises a cancer cell-binding strand having a sequence consisting of SEQ ID NO: 43-62 or 107-115.

In some embodiments, the method comprises synthesizing (e.g., chemically synthesizing) an immune effector cell-binding strand comprising a nucleic acid sequence that is at least 60% identical (e.g., at least 65% identical, at least 70% identical, at least 75% identical, at least 80% identical, at least 85% identical, at least 90% identical, at least 92% identical, at least 94% identical, at least 96% identical, at least 98% identical) to any one of SEQ ID NOs: 1-42, 88-106 or 116. In some embodiments, the method comprises synthesizing an immune effector cell-binding strand comprising a nucleic acid sequence of any one of SEQ ID NOs: 1-42, 88-106 or 116. In certain embodiments, the method comprises synthesizing an immune effector cell-binding strand comprising a nucleic acid sequence that comprises at least at least 20 (e.g., at least 25, at least 30, at least 35, at least 40, at least 45, at least 50) consecutive nucleotides of any one of SEQ ID NO: 1-42, 88-106 or 116. In some embodiments, the method comprises synthesizing an immune effector cell-binding strand having a sequence consisting essentially of SEQ ID NOs: 1-42, 88-106 or 116. In certain embodiments, the method comprises synthesizing a nucleic acid having a sequence consisting of SEQ ID NOs: 1-42, 88-106 or 116.

In some embodiments, the synthesized cancer cell-binding strand and the synthesized immune effector cell-binding strand further comprise complementary 5′ sequences. In some embodiments, the step (3) comprises hybridizing the synthesized cancer cell-binding strand and the synthesized immune effector cell-binding strand. In some embodiments, the complementary 5′ sequence comprising a CpG-motif.

In some embodiments, the complementary 5′ sequence comprises a nucleic acid sequence that is at least 60% identical (e.g., at least 65% identical, at least 70% identical, at least 75% identical, at least 80% identical, at least 85% identical, at least 90% identical, at least 92% identical, at least 94% identical, at least 96% identical, at least 98% identical) to any one of SEQ ID NOs: 63-66. In some embodiments, the complementary 5′ sequence comprises a nucleic acid sequence of any one of SEQ ID NOs: 63-66. In certain embodiments, the complementary 5′ sequence comprises a nucleic acid sequence that comprises at least 12 (e.g., at least 13, at least 14, at least 15, at least 16, at least 17, at least 18, at least 19, at least 20, at least 21, at least 22) consecutive nucleotides of any one of SEQ ID NO: 63-66. In some embodiments, the complementary 5′ sequence has a sequence consisting essentially of SEQ ID NOs: 63-66. In certain embodiments, the complementary 5′ sequence has a sequence consisting of SEQ ID NO: 63-66. In certain embodiments, the double-stranded CpG motif comprises a partial PS modification.

In certain aspects, provided herein is a method of treating cancer in a subject comprising administering to the subject a bispecific personalized aptamer made with the method described herein.

TABLE 1

SEQ ID numbers

| Category | Aptamer name | SEQ ID NO: | Sequence 5′ to 3′ |
|---|---|---|---|
| T cell engager | CTL1 | 1 | TACGCGCAATTCGCCTTGTCGGTGATCTTCCTTTGAACTTGGGCAGT CTG |
| | CTL2 | 2 | TGGCCTGGCCGTGTCGTCTGCTTTATAGTCGGTGATCCCTTGTGTTA ATT |
| | CTL3 | 3 | GCATACCTTTCGTATGCCTTTTTGACCCGTATTTTTGCCCTACCCTTC GG |
| | CTL4 | 4 | TTTTTCGCTATCCAACCCTTCTTTCCAGCCTGCCAATCAGTCGGTGA TCA |
| | CTL5 | 5 | AGGGCAGTCCTGTATCTTAACATTCTCCTACATCCGTAAGTCGGTGA TCC |
| | CTL6 | 6 | GGGCTAAGAGTCTCTATTGTCGGCAGTCGTCTAATATTTCCCGTCCA ATT |
| | CTL7 | 7 | ACTTCCGGTGATTTGATTTCACTTCCTGGGCAGTCAATGTGATTCTC TAC |
| | CTL8 | 8 | GGTCAGTCGCCTTTGTCGGTGATGTACTCGCGCAGTCGGGTTCCCCT TAA |
| | CTL9 | 9 | GGGTCTGTTGGTCCTAGGGCAGTCGTACTTCTAATTCTTGTCCCGAT GAT |
| | CTL10 | 10 | CTTGTCGGTGATCTATAGTCGGTGATATATTTTGTCCTATGGTAGTC GAT |
| | CTL11 | 11 | GGGCTCATGGGCAGTCTTTTTACTACCTCCTATTTACGTATCCCGCT CCT |
| | CTL12 | 12 | CACCCGCGCATTTCCCCCCAGTCGGTGATTCTTATATGTACCTGTTC CTC |
| | CTL13 | 13 | GGGCACGTCCATTCGCGTTTTTGTTCCGTTTCTCCCTTTTTGGATTTT GC |
| | CTL14 | 14 | CAGTCGGTGTCACTCCAGCGGTCGGTTCACTCCACATTCTCCCATCT GTC |
| | CTL15 | 15 | GGCAGTCACCATTCTCTTTGGGCAGATTGTCTCTCATCCATATGTCT CCT |
| | CTL16 | 16 | CTACCTCCTTAGTCGGTGATTCGATCTATGGGCCTAACTGCCTTCTC TGT |
| | CTL17 | 17 | GGGATGCGGGGCCCCGTTCTTTTTGTCTCTCATTTTGTCACTTTTTTT GT |
| | CTL18 | 18 | GGTCAGTCCCTTCGGCATGTCGGGATTCCCTCTTTTCGCCTCGTTTCT TT |
| | CTL19 | 19 | GGCTGTCGAACTTTCTCCCTCCCACCGCAGTCGGCCCCTCATCAGTC GTA |
| | CTL20 | 20 | ACTTCCGGTGATTTGATTTCACTTCCTGGGCAGTCAATGTGATTCTC TAC |
| | CTL21 | 21 | ACGTCTGTCGGTGACCTGTAATAGTTTATGTCGGTGATACAGCTTTC CCT |
| | CTL22 | 22 | CTGTCGGTGATCATATAACGCAGTCGGTGTAGTTTAATCCCACTCCC CTA |
| | CTL23 | 23 | GGCCAGTGTCCCAGTCGTGATTGTAATATTAGATTCTTTGTGGCAGT CGT |
| | CTL24 | 24 | ACTCGTCGGTGATTTTAGACCTTTCTCGGTGATCAACACGTCATGCT ATT |
| | CTL25 | 25 | GCCTCGATATCCTCAGGAGTCGGTGTTTCATTCAATCGTCGGTGATA AAT |
| | CTL26 | 26 | GGTCAGTCCGTATACCGCCAATCCGAACCGCAGTCGGTGTCCGCTT TTAC |
| | CTL27 | 27 | TCGGGTTAGATGTCGGTCCCACTATATGTCGGTGATCTAATATTGAA CTT |
| T cell engager (CD3 Binding) | CS6 | 88 | ATCGTATAAGGGCTGCTTAGGATTGCGATAATACGGTCAA |
| | CS7 | 89 | CATTTCATAGGGCTGCTTAGGATTGCGAAGGTAATGCCAG |
| | CS8 | 90 | CCCTTACCCCTTTTAGGTCTGCTTAGGATTGCGAAAAAAG |
| | CS9 | 91 | TTGTAAGGACTGCTTAGGATTGCGAAAACAATATTCGTAT |
| | CS8c | 92 | CTTTTAGGTCTGCTTAGGATTGCGAAAAAAG |
| | Ppos 10 | 93 | TCCATGGGTCTGCTCTAGGATTGCGTTCATGGTCTCCCCG |
| | Ppos 11 | 94 | AATTACAACCTTGGATTGCAAAGGGCTGCTGTGTTGTTTA |
| | Ppos 12 | 95 | ATCGGAGCTGTTCCTTGATACCGATTCAAAAAGTTCGTAC |
| | Ppos 13 | 96 | AATTTGTAGGGACTGCTCAGGATTGCGGATACAAATTAAT |
| | Ppos 14 | 97 | AGACATTGGGGACTGCTCGGGATTGCGAATCTATGTCTCC |
| | Ppos 15 | 98 | CCC111111AACTAGGTCTGCTTAGGATTGCGAATGTTAA |
| | Ppos 16 | 99 | ACCTCAAAAGCGCGGGCTGCTCAAAGGATTGCGTAGCTTT |
| | Ppos 17 | 100 | GGGGGTTAAGGGCTGCTTAGGATTGCGATAATACGGTCAA |
| | Ppos 18 | 101 | AACATATAACTGCTCAATAATATAGATAAAATACTCACAA |
| | CS1 | 102 | CTCTACCTGACTGTAACCTCTCGCTCCCCCCCATTCGCGC |
| | CS2 | 103 | TTGTCCCTCTACGCCGCCCTTTACTACCACTCCTGCGATT |
| | CS3 | 104 | TCCAGCACACCGACCGCCCCTCTACATTACCCCCTGGACT |
| | CS4 | 105 | CCCCTCCATTCCCCCGCCTCGTCCACCCTACTCCTTAGTC |
| | CS5 | 106 | CATCGACGCCCACACACCACTTCCCGTTCCCCTGCATCAT |
| | CpG1\|CTL3 | 28 | TCGTCGTCGCGGTTCGCGTCCGTGCATACCTTTCGTATGCCTTTTTG ACCCGTATTTTTGCCCTACCCTTCGG |
| CpG motif - T cell Engager strand | 5PS-CpG1\|CTL3 | 29 | T*C*G*T*C*GTCGCGGTTCGCGTCCGTGCATACCTTTCGTATGCCTTT TTGACCCGTATTTTTGCCCTACCCTTCGG |
| | 10PS-CpG1\|CTL3 | 30 | T*C*G*T*C*GTCGCGGTTCGCG*T*C*C*G*TGCATACCTTTCGTATGC CTTTTTGACCCGTATTTTTGCCCTACCCTTCGG |
| | FullPS-CpG1\|CTL3 | 31 | T*C*G*T*C*G*T*C*G*C*G*G*T*T*C*G*C*G*T*C*C*G*TGCATACCT TTCGTATGCCTTTTTGACCCGTATTTTTGCCCTACCCTTCGG |
| | CpG1\|CTL6 | 32 | TCGTCGTCGCGGTTCGCGTCCGTGGGCTAAGAGTCTCTATTGTCGGC AGTCGTCTAATATTTCCCGTCCAATT |
| | CpG1\|CTL5 | 33 | TCGTCGTCGCGGTTCGCGTCCGTAGGGCAGTCCTGTATCTTAACATT CTCCTACATCCGTAAGTCGGTGATCC |
| | CpG1-CS6 | 116 | TCGTCGTCGCGGTTCGCGTCCGTATCGTATAAGGGCTGCTTAGGATT GCGATAATACGGTCAA |
| Non-CpG 22b complem entary seq - T cell engager strand | Non-CpG\|CTL3 | 34 | CTTAATCAGACATTATACAAATTGCATACCTTTCGTATGCCTTTTTG ACCCGTATTTTTGCCCTACCCTTCGG |
| | Non-CpG\|CTL6 | 35 | CTTAATCAGACATTATACAAATTGGGCTAAGAGTCTCTATTGTCGGC AGTCGTCTAATATTTCCCGTCCAATT |
| | Non-CpG \| CTL5 | 36 | CTTAATCAGACATTATACAAATTAGGGCAGTCCTGTATCTTAACATT CTCCTACATCCGTAAGTCGGTGATCC |
| Non-CpG 18b complem entary seq-T cell engagers | Non CpG 18b\|CTL3 | 37 | GAATTAACAATTATAACGTTTGCATACCTTTCGTATGCCTTTTTGAC CCGTATTTTTGCCCTACCCTTCGG |
| | Non CpG 18b\|CTL5 | 38 | GAATTAACAATTATAACGTTTAGGGCAGTCCTGTATCTTAACATTCT CCTACATCCGTAAGTCGGTGATCC |
| | Non CpG 18b\|CTL6 | 39 | GAATTAACAATTATAACGTGGGCTAAGAGTCTCTATTGTCGGCAGT CGTCTAATATTTCCCGTCCAATT |
| NK cell engager all forms | CD16 | 40 | CCACTGCGGGGGTCTATACGTGAGGAAGAAGTGG |
| | CpG1\|CD16 | 41 | TCGTCGTCGGCGTTCGCGTCCGTCCACTGCGGGGGTCTATACGTGA GGAAGAAGTGG |
| | Non-CpG 18b\|CD16 | 42 | GAATTAACAATTATAACGTCCACTGCGGGGGTCTATACGTGAGGAA GAAGTGG |
| | HCT116-VS6 | 43 | TCCTTGTCAGCACTTTCAGAGCACTTTCCCGTAGAACTTAAGGGACA TGC |
| Cancer -Cell Targeting Variable Strands | HCT116-VS12 | 44 | GATTGATCTATTTTCCATATCGCGTTGAGTGTAAAGCCACGAAGGG TTAT |
| | MCF7-VS13 | 45 | ATTGGAGTTTTCCAATCAGAAAGGATTCGGTCAGCTGCAC |
| | MCF7-VS16 | 46 | TGGAAACAGCTGCAACTTTTCTGGGACGTGAATGCCTCGC |
| | MCF7-VS19 | 47 | ACTCAAAAATTAGGCAGGTGTAAGTATAACTCGTGCCTGC |
| | A549-VS3 | 107 | GCAGGCGGAAAATGTCAGGGCACGTTGGTCACGTATTTTT |
| | A549-VS20 | 108 | AGCAATCATATGGCTGTGCTCATTTAATAAGCAAGCTGGG |
| | A549-VS45 | 112 | GTGTTAGTGATGCGAGCTCCTTACCATTAGATAGAGGCTG |
| | CRC13-VS31 | 113 | GCTGCGTCCTCCATTAGCGCTGAGACTTACATTCCTATAC |
| | CRC13-VS48 | 114 | TCCAAGCATAGGACGATACCTTGCATTTCCTTTTCAGATC |
| | CRC13-VS81 | 115 | GGTATCTTTTCTTCGTCCATTACTATCGGTGTTCGAACTC |
| CpG motif-Variable Strand | CpG1′\|HCT11 6-VS6 | 48 | CGGACGCGAACGCCGACGACGATTCCTTGTCAGCACTTTCAGAGCA CTTTCCCGTAGAACTTAAGGGACATGC |
| | CpG1′\|HCT11 6-VS12 | 49 | CGGACGCGAACCGCGACGACGATGATTGATCTATTTTCCATATCGC GTTGAGTGTAAAGCCACGAAGGGTTAT |
| | 5PS-CpG1′\|HCT1 16-VS12 | 50 | C*G*G*A*C*GCGAACCGCGACGACGATGATTGATCTATTTTCCATAT CGCGTTGAGTGTAAAGCCACGAAGGGTTAT |
| | 10PS-CpG1′\|HCT1 16-VS12 | 51 | C*G*G*A*C*GCGAACCGCGACG*A*C*G*A*TGATTGATCTATTTTCC ATATCGCGTTGAGTGTAAAGCCACGAAGGGTTAT |
| | FullPS-CpG1′\|HCT1 16-VS12 | 52 | C*G*G*A*C*G*C*G*A*A*C*C*G*C*G*A*C*G*A*C*G*A*TGATTGAT CTATTTTCCATATCGCGTTGAGTGTAAAGCCACGAAGGGTTAT |
| | CpG1′\|MCF7-VS13 | 53 | CGGACGCGAACCGCGACGACGATATTGGAGTTTTCCAATCAGAAAG GATTCGGTCAGCTGCAC |
| | CpG1′\|MCF7-VS16 | 54 | CGGACGCGAACCGCGACGACGATTGGAAACAGCTGCAACTTTTCTG GGACGTGAATGCCTCGC |
| | CpG1′\|MCF7-VS19 | 55 | CGGACGCGAACCGCGACGACGATACTCAAAAATTAGGCAGGTGTA AGTATAACTCGTGCCTGC |
| | CpG1′\|A549-VS3 | 109 | C*G*G*A*C*GCGAACCGCGACGACGATGCAGGCGGAAAATGTCAG GGCACGTTGGTCACGTATTTTT |
| | CpG1′\|A549-VS20 | 110 | C*G*G*A*C*GCGAACCGCGACGACGATAGCAATCATATGGCTGTGC TCATTTAATAAGCAAGCTGGG |
| | CpG1′\|4T1-VS32 | 111 | CGGACGCGAACCGCGACGACGATAAACTCTATCGTCCAGAGAGAA TGTCTGCCTACTGATTTG |
| Non-CpG 22b complem entary seq-Variable Strand | Non-CpG′ \| HCT116-VS6 | 56 | ATTTGTATAATGTCTGATTAAG T TCCTTGTCAGCACTTTCAGAGCACTTTCCCGTAGAACTTAAGGGACA TGC |
| | Non-CpG′\| HCT116-VS12 | 57 | ATTTGTATAATGTCTGATTAAGTGATTGATCTATTTTCCATATCGCG TTGAGTGTAAAGCCACGAAGGGTTAT |
| | Non-CpG′\| MCF7-VS13 | 58 | ATTTGTATAATGTCTGATTAAGTATTGGAGTTTTCCAATCAGAAAGG ATTCGGTCAGCTGCAC |
| | Non-CpG′\| VS16 | 59 | ATTTGTATAATGTCTGATTAAGTTGGAAACAGCTGCAACTTTTCTGG GACGTGAATGCCTCGC |
| | Non-CpG′\| VS19 | 60 | ATTTGTATAATGTCTGATTAAGTACTCAAAAATTAGGCAGGTGTAA GTATAACTCGTGCCTGC |
| Non-CpG 18b complem entary seq-Variable Strand | Non CpG 18b′ \|HCT116 VS6 | 61 | CGTTATAATTGTTAATTCTTCCTTGTCAGCACTTTCAGAGCACTTTCC CGTAGAACTTAAGGGACATGC |
| | Non CpG 18b′ \|HCT116 VS12 | 62 | CGTTATAATTGTTAATTCTGATTGATCTATTTTCCATATCGCGTTGA GTGTAAAGCCACGAAGGGTTAT |
| CpG motifs | CpG1 | 63 | TCGTCGTCGCGGTTCGCGTCCG |
| | CpG1′ | 64 | CGGACGCGAACGCCGACGACGA |
| | CpG2 | 65 | CGTCGTCGGTCGTCGTCGCTCG |
| | CpG2′ | 66 | CGAGCGACGACGACCGACGACG |

### BRIEF DESCRIPTION OF FIGURES

FIG. 1 is schematic representation of a bispecific personalized aptamer showing the three different domains.

FIG. 2 depicts the personalized aptamer selection process funnel.

FIGS. 3A - 3D show three modes-of-actions (MoAs) in solid tumors for an intratumorally administered bispecific personalized aptamer (FIGS. 3A-3C) and its downstream systemic effect (FIG. 3D).

FIG. 4 shows critical steps in the personalized process for each patient.

FIG. 5 shows the scheme of CTL Binding Cell-SELEX process. Rounds 1 and 2 were done using cells of donor#1 (labelled in blue). Rounds 3, 4, and 6 were done using cells from donor#2 (labelled in cyan). Negative selection was done after rounds 3 and 4 with CD8 - negative cells of donor #1 and donor#2, respectively. The final round, round 7, was repeated three times: one time at “normal” conditions (i.e., 3x wash & short incubation time), one time with long incubation time before the last wash (“long wash”) and finally with twice the number of washes (“6x wash”). Round 7 was done using cells from donor #3.

FIGS. 6A and 6B show the binding SELEX comparative assay. Isolated CD8 T cells were incubated either with the random library 2.6, or with one of the binding SELEX outcome of rounds 4, 6 or 7 tagged with Cy-5 for 1 hour at 37° C. Cy-5 fluorescence intensity was assayed using flow cytometry. FIG. 6A shows the histograms of Cy-5 fluorescence intensity of each round. FIG. 6B shows the fold change of each round over the initial library random 2.6 library.

FIGS. 7A-7D show next generation sequencing (NGS) analysis results. FIG. 7A shows relative abundance of individual sequences in the different rounds sequenced (R2, R5, R6 and R7). Top 100 most abundant sequences of the final enriched library R7 are displayed in grey. Top 10 most abundant sequences are displayed in color. FIG. 7B shows R7 bound-to-unbound ratio of individual sequences identified following the “long wash” stringency plotted against relative abundance in R7. Selected sequences are shown in color. FIG. 7C shows R7 bound-to-unbound ratio of individual sequences in the 6x wash stringency plotted against relative abundance in R7. Selected sequences are shown in color. FIG. 7D shows R7 bound-to-unbound ratio of individual sequences in the “long wash” stringency plotted against R7 bound-to-unbound ratio of individual sequences in the 6x wash stringency.

FIG. 8 shows the initial screen of putative aptamers for binding to CD8 cells via flow cytometry. Isolated T cell fluorescence was measured after each wash cycle for a total of three washes. Results were normalized to the “random” aptamer in each wash. N= 1 or 2.

FIG. 9 depicts promising CD8 cell binding candidate, CTL3, predicted structure by NUPACK (Zadeh et al. (2011) J. Comput. Chem. 32:170-173).

FIG. 10 shows that CTL3 binds PBMCs. CTL3 aptamer exhibited significantly higher binding affinity to total PBMCs compared with control aptamers. Cy-5 labelled CTL3, random aptamer sequence (RND) and Poly T aptamers each at 250 nM, were tested for their binding post 1 hour (hr) incubation at 4° C. Unstained cells represented cells without aptamer. N=3.

FIGS. 11A-11D show CTL3 binding to different PBMC sub-populations. CTL3 bound to lymphocytes while no significant binding to monocytes was observed (FIGS. 11A and 11B). CTL3 bound to CD8 positive and negative cells equally (FIGS. 11C and 11D). Cy-5 labelled CTL3, RND and Poly T aptamers each at 250 nM, were tested for their binding following 1 hr incubation at 4° C. Unstained cells represented cells without aptamer. N=3.

FIGS. 12A and 12B show CTL3 binding compared with the scrambled sequence. CTL3 aptamer exhibited significantly binding affinity to PBMC (FIG. 12A) and to CD8 T cells and (FIG. 12B) compared with control scrambled (SCR) aptamer. Cy-5 labelled CTL3 and CTL3 SCR aptamers each at 250 nM, were tested for their binding following 1 hr incubation at 4° C. Unstained cells represent cells without aptamer. N=3.

FIG. 13 shows that CTL3 bound to isolated CD8 T cells. Cy-5 labelled CTL3, RND and Poly T aptamers each at 250 nM, were tested for their binding to isolated CD8 cells following 1 hr incubation at 4° C. Unstained cells represented cells without aptamer.

FIGS. 14A and 14B show that CTL3 bound to activated and expended Pan-T cells. CTL3, RND and Poly T aptamers, were tested for their binding to activated and expanded Pan-T cells at day 11 post-initial activation. CTL3 bound both CD8 positive (FIG. 14A) and negative cells (FIG. 14B) as compared with control aptamers. Cy-5 labelled CTL3, RND and Poly T aptamers each at 250 nM, were tested after 1 hr incubation at 4° C. Unstained cells represented cells without aptamer. N=1.

FIG. 15 shows Integral Molecular’s Membrane Proteome Array (MPA) description. MPA is a high-throughput cell-based platform for identifying the membrane protein targets of ligands. Membrane proteins were expressed in human cells on 384-well microplates, and ligand binding was detected by flow cytometry, allowing sensitive detection of both specific and off-target binding.

FIG. 16 shows the membrane protein array screening with CTL3.

FIG. 17 shows target hit validation for CTL3 aptamer by sequential dilution.

FIG. 18 shows a schematic of thermofluorimetric analysis (TFA) of aptamer-protein binding. Intercalator fluorescence is low in the melted, free state (left) and high in the folded aptamer or protein bound state (middle, right). Protein binding adds stability, increasing aptamer melting temperature (i.e., Tₘ,bound>Tₘ,unbound). FIG. 18 is adapted from Hu, Kim and Easley (2016) HHSPublic Access. 7:7358-7362.

FIG. 19 shows quantitative protein detection with TFA at 100 nM CTL3. Increasing Notch2 concentration and increasing CD160 concentrations were used as control. Total fluorescence (left) and fluorescent curve derivative (right) are shown.

FIGS. 20 A - 20C show assessment sequences binding to recombinant Notch2. CTL3 and two scrambled DNA sequences were assessed for their binding to recombinant Notch2.

FIGS. 21A - 21C show Quantitative Protein Binding Detection with TFA. Tₘ profile curves were generated using 100 nM of CS with increasing concentrations of either human recombinant Notch2 (green, FIG. 21A), mouse recombinant Notch2 (purple, FIG. 21B), and rat recombinant Notch2 (orange, FIG. 21C).

FIGS. 22A and 22B show the scheme of CD3ε binding SELEX process.

FIGS. 23A and 23B show the binding SELEX comparative assay. Binding assay was performed on target protein CD3ε-beads complex (black) or control protein IgG1 (gray) with initial random library (Rnd Lib) and library enriched pools from Rounds 3(R3), 6(R6), 9(R9), and 11(R11). Post incubation and wash the library DNA was eluted and concentration in the supernatant was evaluated via real-time-PCR. The standard curve was performed with a random library (top). Binding of Cy5 fluorescently labeled libraries to Jurkat T cell line and to Pan B cells was demonstrated by flow cytometry (FIG. 23B). Dot plots and histogram graphs are shown. Flow data quantification of Cy5 median fluorescence intensity (MFI) are shown.

FIGS. 24A-24C show next generation sequencing (NGS) analysis results. FIG. 24A shows analysis of single aptamer sequences from 8^{th}, 9^{th}, 10^{th}, and 11^{th} SELEX rounds enriched libraries on dot plot where the X-axis represents mean P-negative and the Y-axis represents mean P-positive. The diagonal line represents the threshold between specific-binder aptamers and low, nonspecific, binding aptamer sequences. Top 5 candidates selected for further examination are indicated with their names. FIG. 24B shows sequences LOGO display of the shared motif (using GLAM2 software) of top 14 specific-binder aptamers (upper) and top 4 selected aptamers (lower). FIG. 24C shows secondary structural analysis (mfold) of the 5 selected candidates. Motif nucleotides location are marked with a red asterisk.

FIG. 25 shows aptamer sequences binding to target protein by HPLC. Folded and Cy5-labelled aptamer candidates were assayed for recombinant Human CD3ε (hCD3ε) binding. Aptamers were incubated for 1 hr at 37° C. with hCD3e or with the negative control IgG1. PolyT was used as a negative control sequence.

FIGS. 26A-26C show CS6 binding to T cells as demonstrated via flow cytometry. Jurkat cells and Kasumi-1 cells were incubate with CpG′-Cy5 labelled CS6, CS7 and CS8c, and analyzed by flow cytometry (FIG. 26A). Jurkat cells and Daudi cells were incubate with CpG′-Cy5 labelled CS6, CS7 and CS8c and analyzed by flow cytometry. MFI quantification is indicated below (FIG. 26B). Isolated pan T cells and pan B cells were incubated with CpG′-Cy5 labeled CS6 and analyzed by flow cytometry. Representation of dot plots with Cy5 (X-axis)/SSC (Y-axis) of T cells and B cells as well as MFI quantification are presented (FIG. 26C).

FIG. 27 shows CS6 effective concentration. Jurkat cells were incubated with serially-diluted concentrations of CpG′-Cy5 labelled CS6 and analyzed by flow cytometry to determine compound’s EC₅₀.

FIG. 28 shows binding of CS6 either to the target protein hCD3ε (top) or to a non-specific IgG control protein (bottom) by SPR sensogram.

FIG. 29 shows that bispecific aptamer acts as a T cell engager and stimulates CD69 elevation.

FIGS. 30A-30C show schematic representation of bispecific personalized aptamer showing three different domains. The double-stranded hybridization domain functioning as a TLR9-agonist is emphasized (FIG. 30A)The chemical structure of phosphodiester bond compared with phosophrothioate modification (adapted from Pohar et al. (2017) Sci. Rep. 7:14598) (FIG. 30B).Lists of (i) the 22 base pairs (bps) CpG bridge sequences: CpG1 (SEQ ID NO: 63), CpG1′ (SEQ ID NO: 64), CpG2 (SEQ ID NO: 65), and CpG2′ (SEQ ID NO: 66) and (ii) shows PS variations of the bispecific personalized aptamer showing the different monomer sequences: CpG1|CTL3 (SEQ ID NO: 28), 5PS-CpG1|CTL3 (SEQ ID NO: 29), 10PS-CpG1|CTL3 (SEQ ID NO: 30), FullPS-CpG1|CTL3 (SEQ ID NO: 31), CpG1′|VS12 (SEQ ID NO: 49), 5PS-CpG1′|VS12 (SEQ ID NO: 50), 10PS-CpG1′|VS12 (SEQ ID NO: 51), FullPS-CpG1′|VS12 (SEQ ID NO: 52). Phosphodiester backbone is indicated in light gray. PS backbone is indicated by an asterisk (FIG. 30C).

FIGS. 31A and 31 B show the effect of introducing CpG1 motif to the bispecific aptamer on its ability to induce tumor cell death. Killing assay for NK cells and CD8 T cell engagers with CpG-containing bispecific personalized aptamers (FIG. 31A) and with different compositions of PS modifications in which, for example, both CTL3 and VS12 monomers have 5 of PS modifications at their 5′ ends for CTL3|5PS-CpG1-5PS|VS12 (FIG. 31B). HCT116 cells were co-cultured with PBMCs for 72 hours with three doses of 100 µM bispecific personalized aptamers. Lethality was analyzed by flow cytometry on HCT116 cells.

FIGS. 32A-32C show CpG / TLR9 agonistic motif of the bispecific aptamer modulate the immune response in both human and mice. Pan B-cells were isolated and seeded in 96 wells plate (200,000 cells/well) for 24 hrs. Cells were treated with Vehicle, PolyT-PolyT (50 µM) as negative control, 5 µM oligodeoxynucleotide ODN-2395 (Roda et al. (2005) J. Immunol. 175:1619-1627), a cell-culture tested ODN was used as a positive control and with bispecific aptamer CTL3-VS12 (50 µM). Twenty-four hrs post-treatment, cells were collected and analyzed by flow cytometry for CD86 expression. One representative donor out of three is presented (FIG. 32A). Splenocytes from BALB/c mice were isolated (n=3) and seeded in 96 wells plate (500,000 cells/well). Cells were treated with Vehicle, ODN negative control (5 µM), ODN 2395 (5 µM) as positive control and with bispecific aptamer CTL3-VS12 (50 µM) for 48h. Forty-eight hrs post-treatment, cells were centrifuged and supernatant were collected and analyzed for IL-6 secretion using IL-6 ELISA kit (FIG. 32B). PBMCs were co-cultured with HCT-116 cells for 48 hours and treated with 50 µM of ODN 2395 with (positive control) or without (negative control) PS modifications, and with dsCpG2, as a stand-alone sequence or in the context of bispecific aptamer. Cells media were collected and analyzed for IFN-alpha by ELISA kit (FIG. 32C).

FIGS. 33A and 33 B show that the CpG motif (SEQ ID Nos. 63 and 64), either in a single strand form or within bispecific aptamer structure, modulates IL-6 secretion (FIG. 33A) and co-stimulatory molecules expression (FIG. 33B).

FIG. 34 shows that the CpG motif (SEQ ID Nos. 63 and 64), in the context of the bispecific entity, acts in a dose-dependent manner.

FIG. 35 shows functional enrichment of DNA libraries for the activation of apoptosis in HCT116 (colorectal carcinoma) cells.

FIG. 36 shows bioinformatic analysis of the final enriched functional library post-NGS.

FIG. 37 shows multiple-dosing of top aptameric candidates for cytotoxic effect.

FIGS. 38A and 38B show the functional enrichment results for MCF7 cell line. Comparative functional assay showing enriched library for initial round of enrichment (F3.1), fifth (F3.5), sixth (F3.6), and final (F3.7) rounds incubated with MCF7 cell line for 2 hr. Annexin V positive staining was measured via flow cytometry and normalized to the initial round of enrichment (F3.1). Total Annexin V levels are indicated above the bars of first and final rounds of enrichments (FIG. 38A). Sequencing results presented in a scatter plot where each dot represents a single sequence. The X-axis shows the propensity of a sequence to induce Annexin V binding on MCF7 cells (P Positive), and the Y-Axis shows the propensity of a sequence to induce Annexin V binding on negative selection cells, PBMCs from a healthy donor (P Negative). Dots colored in green represent sequences which were selected to be screened individually via high content fluorescence microscopy (FIG. 38B).

FIGS. 39A and 39B show the high-content screening of individual aptamers by time-lapsed fluorescent microscopy. Representative images at t = 14 hrs from initial screen of aptamer leads VS13 (SEQ ID NO: 45), VS16 (SEQ ID NO: 46) and VS19 (SEQ ID NO: 47) all in 50 µM concentration, compared with Vehicle, Random Oligonucleotide and Staurosporine. Cell nuclei were stained by Hoechst33258 (blue), Annexin V (pink) (FIG. 39A). Scatter plot depicts analysis of the lead aptamer. The X-axis shows the total percent of cells positive for Annexin V at t =14 hrs. The Y-axis shows the fold over increase of Annexin V at t = 14 hrs relative to t = 0 hrs for each aptamer. Top leads are marked in pink, negative controls in green and positive control (Staurosporine) in red (FIG. 39B).

FIGS. 40A and 40B show potency and specificity confirmation for final MCF7 versus aptamer leads. Dose-dependent (50, 100, and 200 µM) viability of MCF7 cells incubated with lead aptamers (red line) VS13 (right panel) and VS16 (left panel) were assessed for 48 hrs and compared with PolyT aptamer control (dashed line) and PBMCs (blue line), dose administered daily. Viability was measured using the XTT assay and plotted as fold over Vehicle control (Y-axis) (FIG. 40A). Scatter plot summary showing MCF7 viability (Y-axis) vs. PBMC viability (X-axis) for lead aptamers was tested. The positive control (Staurosporine) is indicated by a red circle. Vehicle and Untreated controls are indicated by light green circles. Six lead aptamers are indicated in the dark blue hexagons for the 200 µM dose, blue diamonds for 100 µM dose and light blue triangles for 50 µM dose level. The PolyT control is indicated by dark green symbols: hexagon, diamond, and triangle for 200 µM, 100 µM, and 50 µM respectively. VS13 and VS16 are indicated by “13” and “16” (FIG. 40B).

FIG. 41 shows functional enrichment results for A549 cell line.

FIG. 42 shows potency confirmation for final A549 Variable Strand aptamer leads.

FIG. 43 shows CRC organoids formation.

FIGS. 44A and 44B show functional enrichment results for CRC13 organoids (FIG. 44A) and potency confirmation for final CRC13 Variable Strand aptamer leads (FIG. 44B)

FIG. 45 shows schematic description of bispecific personalized aptamers formulation, using CTL3|CpG1|VS12 example. Each arm is reconstituted to a concentration of 2 mM and undergoes aptamer folding by a rapid temperature ramp, i.e. instant cooling of the solution from 95° C. to 4° C., followed by mixing and hybridization to yield a bispecific entity with a final concentration of 1 mM.

FIGS. 46A and 46B show cytotoxic assay mediated by bispecific personalized aptamers engaging either natural killer (NK) cells or cytotoxic T lymphocytes (CTLs). HCT116 cells and peripheral blood mononuclear cells (PBMCs) from two healthy donors were co-cultured for 72 h. Natural killer and CTL bispecific personalized aptamers were administered daily at 100 µM for a total of three doses followed by Live/Dead dye assay. FIG. 46A shows the lethality of HCT116 cells and FIG. 46B shows the lethality of PBMCs. Vehicle and polyT||polyT dimer are used as negative controls. Mitomycin (10 µM) and anti-CD3/anti-CD28 antibodies (1 µg/mL), administered in a single dose, are positive controls. n=2.

FIG. 47 shows bispecific personalized aptamers targeting cancer cells in a dose-dependent manner. Four concentrations of each bispecific personalized aptamer were tested: 10, 25, 50 and 100 µM. HCT116 cells were co-cultured with PBMCs and for 72 hours in the presence of bispecific personalized aptamers at the indicated concentrations. Lethality was analyzed by flow cytometry. n=2.

FIGS. 48A and 48B show killing assay data for bispecific personalized aptamers with PBMCs and HCT116 or MCF10a cells. Either HCT116 or MCF10a cells were co-cultured with PBMCs for 72 hours. CTL bispecific personalized aptamers were administered daily at 100 µM for a total of three doses followed by Live/Dead dye assay. Lethality was analyzed by flow cytometry. Benchmark criteria for bispecific personalized aptamer selection is emphasized via rectangle.

FIGS. 49A and 49B shows that a bispecific personalized aptamer induced higher lethality than each monomer. HCT116 cells were co-culture with PBMCs for 72 hours with three doses of 100 µM bispecific personalized aptamers or monomers. Lethality was analyzed by flow cytometry on HCT116 cells (FIG. 49A) and PBMCs (FIG. 49B). n=14.

FIG. 50 shows killing assay data for CTL3||VS12 and CTL6||VS12 bispecific personalized aptamers. HCT116 cells were co-cultured with PBMCs for 72 hours with three doses of 100 µM bispecific personalized aptamers or monomers. Lethality was analyzed by flow cytometry. n=3.

FIG. 51 shows that bispecific personalized aptamer induces tumor cell death in vitro.

FIGS. 52A and 52B show that bispecific personalized aptamers induced cytotoxicity in MCF7 cells, co-cultured with PBMCs. PBMCs were primed with anti-CD3 and anti-CD28 antibodies in the presence of IL-2 (400 U/mL) for 4 days prior to co-culture setup. Primed immune cells were co-cultured with MCF7 cells in a 5:1 effector: target ratio and incubated with 100 µM Bispecific Aptamers CTL3||VS13, CTL3||VS16 and CTL3||VS19 for 48 hrs. PolyT dimer (PolyT||PolyT) and Vehicle were used as negative controls. Lethality was measured via Live-Dead Zombie stain (flow cytometry) (FIG. 52A). Viability was measured via XTT and normalized to vehicle control (FIG. 52B). n=4 PBMC donors.

FIGS. 53A-53C show the in vivo efficacy of the CD16||VS12 bispecific personalized aptamer. Female immune-deficient female NOD scid gamma (NSG®) mice were implanted subcutaneously (SC) with HCT116 tumor cells admixed with human PBMCs followed by treatments with 100 mg/kg polyT or 100 mg/kg NK engager bispecific personalized aptamers for a total of twelve doses (marked as priming doses and in triangles) administered SC. Tumor volume was measured through Day 32, mean ± SEM is shown (FIG. 53A). Tumor weight was assessed at end of in-life (Day 33). Results are represented as mean ± SEM. (FIG. 53B). FIG. 13C shows the Kaplan-Meier survival analysis of the bispesific personalized aptamer (FIG. 53C). * indicates significant difference (p ≤ 0.05) and ** (p ≤ 0.01)

FIG. 54 shows the in vivo efficacy of the CTL6||VS12 bispecific personalized aptamer, manufactured by two different vendors. Female NSG® mice were implanted SC with HCT-116 tumor cells admixed with human PBMC followed by a treatment with 100 mg/kg T cell engager bispecific personalized aptamers for a total of twelve doses (marked as a rectangle) administered SC. HCT116 tumor volume was measured through Day 27 (mean ± S.E.M is shown). * indicates significant difference (p ≤ 0.05).

FIGS. 55A and 55B show individual HCT116 tumor volumes of vehicle- and CTL6||VS12-treated mice. Empty shapes represent death.

FIG. 56 shows HCT116 tumor volume on Day 27. Comparison between the different treatment groups. * indicates significant difference (p ≤ 0.05).

FIGS. 57A and 57B show HCT116 tumor volume for CTL3||VS12 treatment, PolyT||PolyT, Vehicle and Untreated mice groups, monitored during the 22 days of the study (FIG. 57A). Tumors were weighted at the end of the in-life phase (FIG. 57B). Statistical T- test was implemented. ** indicates significant difference (p ≤ 0.005) and *** ((p ≤ 0.001)

FIG. 58 depicts Kaplan-Meier survival analysis of CTL3||VS12 treated Mice.

FIGS. 59A and 59B show in vivo efficacy of the exemplary bispecific T cell engager aptamer, comprised of CS6 aptamer (SEQ ID NO: 116) hybridized to HCT116, colon carcinoma cell line-targeting aptamer sequence (named VS12; SEQ ID NO: 50). Female NSG mice were implanted SC with HCT-116 tumor cells admixed with human PBMC followed by a treatment with T cell engager bispecific personalized aptamers for a total of 10 doses administered SC. HCT116 tumor volume was monitored for CS6-VS12 treatment, PolyT-PolyT (non-specific DNA aptamer) and Vehicle mice groups (FIG. 59A).Individual mice growth curves are depicted in FIG. 59B. *** indicates significant difference ((p ≤ 0.001).

FIG. 60 depicts Kaplan-Meier survival analysis of treated Mice. ** indicates significant difference ((p ≤ 0.01).

FIGS. 61A and 61B show the in vivo efficacy of CTL3|5PS-CpG1| VS16 (CTL3-VS16) in xenograft MCF7 tumor model. MCF7 tumor volume was measured for 18 days following CTL3-VS16 or Vehicle treatments. Tumor mean volume ± SEM is presented (n=6) (FIG. 61A) Individual tumor volume increase relative to randomization day is plotted (FIG. 61B) Statistical T- test was implemented. ** indicates significant difference (p ≤ 0.005).

FIGS. 62A and 62B show in vivo efficacy of the exemplary bispecific T cell engager aptamer, comprised of CS6 aptamer (SEQ ID NO: 116) hybridized to 4T1, mammary carcinoma cell line-targeting aptamer sequence (named VS32; SEQ ID NO: 111). Female Balb/c mice were implanted SC with 4T1 tumor cells on both flanks of the mouse. Once the primary tumor has reached a size of 50 mm³, a treatment with T cell engager bispecific personalized aptamers commenced using intratumoral route of administration. Primary and secondary tumor volumes were monitored for CS6-VS12 treatment with or without combination with anti-PD1.

### DETAILED DESCRIPTION

### General

The methods and composition provided herein are based, in part, on the development of bispecific personalized aptamer entities that are composed of two arms. One aptameric arm is variable across different patients and designed to bind to unique targets on the surface of patients’ tumor cells. The second aptameric arm is designed to engage effector immune cells to cause tumor cell lysis. This latter immune-modulating arm is designed to be shared across different patients. In some embodiments, the two arms are bridged by double-stranded DNA. This DNA “bridge” may have toll-like receptor 9 (TLR9) agonistic activity, which leads to increased uptake and engulfment of tumor antigens by antigen presenting cells as well as secretion of pro-inflammatory cytokines. The aptamer’s specificity coupled with effector cell engagement and the TLR9 agonistic activity makes bispecific personalized aptamers promising candidates for a multi-faceted approach to treating cancers. The platform described herein also yields patient-tailored cancer therapeutics to treat patients with individualized solutions.

Therefore, in certain aspects, provided herein are bispecific personalized aptamers that comprise a cancer cell-binding strand that selectively binds to and/or selectively kills cancer cells (e.g., breast cancer cells or colorectal carcinoma cells), including by inducing apoptosis, ICD, necrosis, necroptosis and/or autophagy. The bispecific personalized aptamers also comprise an immune effector cell-binding strand that mediates cancer cell lysis through T cell or NK cell-mediated cytotoxicity. In some embodiments, the cancer cell-binding strand is linked to the immune effector cell-binding strand by a CpG motif that induces TLR9-mediated antigen presenting cell (APCs) stimulation and/or increased uptake of tumor antigens. In some aspects, provided herein are pharmaceutical compositions comprising such bispecific personalized aptamers, methods of using such bispecific personalized aptamers to treat cancer and/or to kill cancer cells and methods of making such bispecific personalized aptamers.

### Definitions

For convenience, certain terms employed in the specification, examples, and appended claims are collected here.

The articles “a” and “an” are used herein to refer to one or to more than one (e.g., to at least one) of the grammatical object of the article. By way of example, “an element” means one element or more than one element.

As used herein, the term “aptamer” refers to a short (e.g., less than 200 bases), single stranded nucleic acid molecule (ssDNA and/or ssRNA) able to specifically bind to a target molecule (e.g. protein or peptide, or to a topographic feature on a target cell.

The term “binding” or “interacting” refers to an association, which may be a stable association, between two molecules, e.g., between an aptamer and target, e.g., due to, for example, electrostatic, hydrophobic, ionic, pi-stacking, coordinative, van der Waals, covalent and/or hydrogen-bond interactions under physiological conditions.

As used herein, two nucleic acid sequences “complement” one another or are “complementary” to one another if they base pair one another at each position.

The term “modulation” or “modulate”, when used in reference to a functional property or biological activity or process (e.g., enzyme activity or receptor binding), refers to the capacity to either up regulate (e.g., activate or stimulate), down regulate (e.g., inhibit or suppress) or otherwise change a quality of such property, activity, or process. In certain instances, such regulation may be contingent on the occurrence of a specific event, such as activation of a signal transduction pathway, and/or may be manifest only in particular cell types.

As used herein, “specific binding” refers to the ability of an aptamer to bind to a single target. Typically, an aptamer specifically binds to its target with an affinity corresponding to a K_{D} of about 10⁻⁷ M or less, about 10⁻⁸ M or less, or about 10⁻⁹ M or less and binds to the target with a K_{D} that is significantly less (e.g., at least 2 fold less, at least 5 fold less, at least 10 fold less, at least 50 fold less, at least 100 fold less, at least 500 fold less, or at least 1000 fold less) than its affinity for binding to a non-specific and unrelated target (e.g., BSA, casein, or an unrelated cell, such as an HEK 293 cell or an E. coli cell).

The term “oligonucleotide” and “nucleic acid molecule” refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, synthetic polynucleotides, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component.

### Bispecific Personalized Aptamers

In certain aspects, provided herein are bispecific personalized aptamers that comprise (a) a cancer cell-binding strand that specifically binds to an antigen expressed on a cancer cell; (b) a CpG motif; and (c) an immune effector cell-binding strand that binds an immune effector cell, wherein the cancer cell-binding strand is linked to the immune effector cell-binding strand by the CpG motif.

In some embodiments, the cancer cell-binding strand is able to induce cell death (e.g., apoptosis) of a cancer cell (e.g., a human cancer cell) when contacted to the cancer cell. In some embodiments, the cancer cell is a patient-derived cancer cell. In some embodiments, the cancer cell is a solid tumor cell (e.g., a breast cancer cell). In certain embodiments, the cancer cell is a carcinoma cell (e.g., a colorectal carcinoma cell). In some embodiments, the aptamers induce cell death when contacted to the cancer cell in vitro. In certain embodiments, the aptamers induce cell death when contacted to the cancer cell in vivo (e.g., in a human and/or an animal model). In some embodiments, the cancer cell-binding strand binds to a cancer antigen selected from Prostate Membrane Antigen (PSMA), Cancer antigen 15-3 (CA-15-3), Carcinoembryonic antigen (CEA), Cancer antigen 125 (CA-125), Tyrosinase, gp100, MART-1/melan-A, HSP70-2-m, HLA-A2-R17OJ, HPV16-E7, MUC-1, HER-2/neu, Mammaglobin-A or MHC-TAA peptide complexes.

In certain embodiments, the cancer cell-binding strand comprises a nucleic acid sequence that is at least 60% identical (e.g., at least 65% identical, at least 70% identical, at least 75% identical, at least 80% identical, at least 85% identical, at least 90% identical, at least 92% identical, at least 94% identical, at least 96% identical, at least 98% identical) to any one of SEQ ID NOs: 43-62 or 107-115. In some embodiments, the cancer cell-binding strand comprises a nucleic acid sequence of any one of SEQ ID NOs: 43-62 or 107-115. In certain embodiments, the cancer cell-binding strand comprises at least 30 (e.g., at least 35, at least 40, at least 45, at least 50, at least 55, at least 56, at least 57, at least 58, at least 59, at least 60, at least 61, at least 62, at least 63, at least 64, at least 65, at least 66, at least 67, at least 68, at least 69) consecutive nucleotides of any one of SEQ ID NO: 43-62 or 107-115. In some embodiments, the cancer cell-binding strand has a sequence consisting essentially of SEQ ID NOs: 43-62 or 107-115. In certain embodiments, the cancer cell-binding strand has a sequence consisting of SEQ ID NO: 43-62 or 107-115.

The terms “identical” or “percent identity,” in the context of two or more nucleic acids, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g. , NCBI web site http://www.ncbi.nlm.nih.gov/BLAST/ or the like).

In certain embodiments, the cancer cell-binding strand is no more than 120 nucleotides in length (e.g., no more than 115 nucleotides in length, no more than 110 nucleotides in length, no more than 105 nucleotides in length, no more than 100 nucleotides in length, no more than 95 nucleotides in length, no more than 90 nucleotides in length, no more than 85 nucleotides in length, no more than 80 nucleotides in length, no more than 75 nucleotides in length, no more than 70 nucleotides in length, no more than 69 nucleotides in length, no more than 68 nucleotides in length, no more than 67 nucleotides in length, no more than 66 nucleotides in length, no more than 65 nucleotides in length, no more than 64 nucleotides in length, or no more than 63 nucleotides in length). In certain embodiment, the cancer cell-binding strand is about 63 nucleotides in length.

In some embodiments, the immune effector cell-binding strand binds to a target expressed by T cell (e.g., CD8+ T cell), B cell, NK cell, macrophage, or dendritic cell. In certain embodiments, the immune effector cell-binding strand binds to an immune effector cell antigen selected from CD16, Notch-2, other Notch family members, KCNK17, CD3, CD28, 4-1BB, CTLA-4, ICOS, CD40L, PD-1, OX40, LFA-1, CD27 PARP16, IGSF9, SLC15A3, WRB and GALR2. In some embodiments, the immune effector cell-binding strand mediates lysis of the cancer cell through T cell or NK cell-mediated cytotoxicity.

In some embodiments, the immune effector cell-binding strand comprises a nucleic acid sequence that is at least 60% identical (e.g., at least 65% identical, at least 70% identical, at least 75% identical, at least 80% identical, at least 85% identical, at least 90% identical, at least 92% identical, at least 94% identical, at least 96% identical, at least 98% identical) to any one of SEQ ID NOs: 1-42, 88-106 or 116. In some embodiments, the immune effector cell-binding strand comprises a nucleic acid sequence of any one of SEQ ID NOs: 1-42, 88-106 or 116.

In certain embodiments, the immune effector cell-binding strand comprises at least 20 (e.g., at least 25, at least 30, at least 35, at least 40, at least 41, at least 42, at least 43, at least 44, at least 45, at least 46, at least 47, at least 48, at least 49, at least 50, at least 51, at least 52, at least 53) consecutive nucleotides of any one of SEQ ID NOs: 1-42, 88-106 or 116. In some embodiments, the immune effector cell-binding strand provided herein has a sequence consisting essentially of SEQ ID NOs: 1-42, 88-106 or 116. In certain embodiments, the immune effector cell-binding strand provided herein has a sequence consisting of SEQ ID NO: 1-42, 88-106 or 116. In certain embodiments, the immune effector cell-binding is no more than 120 nucleotides in length (e.g., no more than 115 nucleotides in length, no more than 110 nucleotides in length, no more than 105 nucleotides in length, no more than 100 nucleotides in length, no more than 95 nucleotides in length, no more than 90 nucleotides in length, no more than 85 nucleotides in length, no more than 80 nucleotides in length, no more than 75 nucleotides in length, no more than 74 nucleotides in length, or no more than 73 nucleotides in length). In certain embodiments, the immune effector cell-binding strand is about 73 nucleotides in length.

The cancer cell-binding strand and the immune effector cell-binding strand may be linked together by hybridization of a 5′ sequence of the cancer cell-binding strand to a 5′ sequence of the immune effector cell-binding strand. In certain embodiments, the 5′ sequence of the cancer cell-binding strand hybridizes to the 5′ sequence of the immune effector cell-binding strand to form a CpG-rich motif, TLR9 agonistic sequence. The cancer cell-binding strand and the immune effector cell-binding strand may be linked together by directly ligating to each of the two ends (e.g., the 5′ ends) of a double-strand sequence. In certain embodiments, the double-strand sequence is a CpG motif, a TLR9 agonist sequence.

In some embodiments, the TLR9 agonist sequence comprises a double-stranded region comprising at least one (e.g., 1, 2, 3, 4, 5, 6, 7, 8, 9, 10, 11, 12, 13, 14, 15, 16, 17, 18, 19, 20) CpG motif nucleotides. In some embodiments, the CpG motif induces TLR9-mediated antigen presenting cell (APCs) stimulation and/or increased uptake of tumor antigens. In some embodiments, the TLR9 agonist sequence induces an anti-tumor response. In some embodiments, the TLR9 agonist sequence induces cytokines production.

In some embodiments, the CPG motif sequence is a double-stranded nucleic acid sequence comprising a sequence that is at least 60% identical (e.g., at least 65% identical, at least 70% identical, at least 75% identical, at least 80% identical, at least 85% identical, at least 90% identical, at least 92% identical, at least 94% identical, at least 96% identical, at least 98% identical) to any one of SEQ ID NOs: 63-66. In some embodiments, the CpG motif sequence is a double-stranded nucleic acid sequence comprising a sequence of any one of SEQ ID NOs: 63-66.

In certain embodiments, the CpG motif sequence is a double-stranded nucleic acid sequence comprising at least 12 (e.g., at least 13, at least 14, at least 15, at least 16, at least 17, at least 18, at least 19, at least 20, at least 21, at least 22) consecutive nucleotides of any one of SEQ ID NO: 63-66. In some embodiments, the CpG motif sequence provided herein has a sequence consisting essentially of SEQ ID NOs: 63-66. In certain embodiments, the CpG motif sequence provided herein has a sequence consisting of SEQ ID NO: 63-66.

In certain embodiments, the CpG motif sequence is no more than 35 nucleotides in length (e.g., no more than 34 nucleotides in length, no more than 33 nucleotides in length, no more than 32 nucleotides in length, no more than 31 nucleotides in length, no more than 30 nucleotides in length, no more than 29 nucleotides in length, no more than 28 nucleotides in length, no more than 27 nucleotides in length, no more than 26 nucleotides in length, no more than 25 nucleotides in length, no more than 24 nucleotides in length, no more than 23 nucleotides in length, or no more than 22 nucleotides in length).

The bispecific personalized aptamer provided herein may comprise any combination of the cancer cell-binding strand and the immune cell-binding strand described herein. For example, in some embodiments, the bispecific personalized aptamer comprises a combination of the cancer cell-binding strand and the immune cell-binding strand selected from the group consisting of: SEQ ID NOs: 29 and 54, 29 and 50, 32 and 50, 33 and 48, 41 and 49, 34 and 59.

In some embodiments, the bispecific personalized aptamers provided herein comprise one or more chemical modifications. Exemplary modifications are provided in Table 2.\

TABLE 2

Exemplary chemical modifications.

| | | | |
|---|---|---|---|
| Terminal | Sugar ring | Nitrogen base | Backbone |
| biotin | 2′-OH (RNA) | BzdU | Phosphorothioate |
| Inverted-dT | 2′-OMe | Naphtyl | Methylphosphorothioate |
| PEG (0.5-40 kDa) | 2′-F | Triptamino | Phosphorodithioate |
| Cholesterol | 2′-NH2 | Isobutyl | Triazole |
| Albumin | LNA | 5-Methyl Cytosine | Amide (PNA) |
| Chitin (0.5-40 kDa) | UNA | Alkyne (dibenzocyclooctyne) | Alkyne (dibenzocyclooctyne) |
| Chitosan (0.5-40 kDa) | 2′-F ANA | Azide | Azide |
| Cellulose (0.5-40 kDa) | L-DNA | Maleimide | Maleimide |
| Terminal amine (alkyne chain with amine) | CeNA | | |
| Alkyl (dibenzocyclooctyne) | TNA | | |
| Azide | HNA | | |
| Thiol | | | |
| Maleimide | | | |
| NHS | | | |

In certain embodiments, the bispecific personalized aptamers comprise a terminal modification. In some embodiments, the bispecific personalized aptamers are chemically modified with poly-ethylene glycol (PEG) (e.g., 0.5-40 kDa) (e.g., attached to the 5′ end of the aptamer). In some embodiments, the bispecific personalized aptamers comprise a 5′ end cap (e.g., is an inverted thymidine, biotin, albumin, chitin, chitosan, cellulose, terminal amine, alkyne, azide, thiol, maleimide, NHS). In certain embodiments, the bispecific personalized aptamers comprise a 3′ end cap (e.g., is an inverted thymidine, biotin, albumin, chitin, chitosan, cellulose, terminal amine, alkyne, azide, thiol, maleimide, NHS).

In certain embodiments, the bispecific personalized aptamers provided herein comprise one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 30 31, 32, 33, 34, 35, 36, 37, 38, 39, 40, 41, 42, 43, 44, 45, 46, 47, 48, 49, 50, 51, 52, 53, or 54) modified sugars. In some embodiments, the bispecific personalized aptamers comprise one or more 2′ sugar substitutions (e.g. a 2′-fluoro, a 2′-amino, or a 2′-O-methyl substitution). In certain embodiments, the bispecific personalized aptamers comprise locked nucleic acid (LNA), unlocked nucleic acid (UNA) and/or 2′deozy-2′fluoro-D-arabinonucleic acid (2′-F ANA) sugars in their backbone.

In certain embodiments, the bispecific personalized aptamers comprise one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 30 31, 32, 33, 34, 35, 36, 37, 38, 39, 40, 41, 42, 43, 44, 45, 46, 47, 48, 49, 50, 51, 52, 53, or 54) methylphosphonate internucleotide bonds and/or phosphorothioate (PS) internucleotide bonds.

In certain embodiments, the bispecific personalized aptamers may comprise PS modification within the double stranded region (e.g., the CpG motif sequence). For example, the double stranded region (e.g., the CpG motif sequence) of the bispecific personalized aptamers may comprise 1, 2, 3, 4, 5, 6, 7, 8, 9, 10, 11, 12, 13, 14, 15, 16, 17, 18, 19, 20, 21, 22 or 23 phosphorothioate (PS) internucleotide bonds on one or both strands. In some embodiments, the double stranded region (e.g., the CpG motif sequence) of the bispecific personalized aptamers may comprise a partial PS modification. In certain embodiments, 5 nucleotides from 5′ ends of the double-stranded CpG motif sequence are modified. In other embodiments, 5 nucleotides from both 5′ and 3′ ends of the double-stranded CpG motif sequence are modified. In certain embodiments, the double-stranded CpG motif sequence comprises a complete PS modification.

In certain embodiments, the aptamers comprise one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 30 31, 32, 33, 34, 35, 36, 37, 38, 39, 40, 41, 42, 43, 44, 45, 46, 47, 48, 49, 50, 51, 52, 53, or 54) triazole internucleotide bonds. In certain embodiments, the aptamers are modified with a cholesterol or a dialkyl lipid (e.g., on their 5′ ends).

In some embodiments, the aptamers comprise one or more modified bases (e.g., BzdU, Naphtyl, Triptamino, Isobutyl, 5-Methyl Cytosine, Alkyne (dibenzocyclooctyne, Azide, Maleimide).

In certain embodiments, the aptamers provided herein are DNA aptamers (e.g., D-DNA aptamers or enantiomer L-DNA aptamers). In some embodiments, the aptamers provided herein are RNA aptamers (e.g., D-RNA aptamers or enantiomer L-RNA aptamers). In some embodiments, the aptamers comprise a mixture of DNA and RNA.

### Pharmaceutical Compositions

In certain aspects, provided herein are pharmaceutical compositions comprising a bispecific personalized aptamer (e.g., a therapeutically effective amount of a bispecific personalized aptamer) provided herein. In some embodiments, the pharmaceutical compositions further comprise a pharmaceutically acceptable carrier. In some embodiments, the pharmaceutical composition is formulated for parenteral administration (e.g., subcutaneous administration).

In certain embodiments, the pharmaceutical composition is for use in treating cancer. In some embodiments, the cancer is a solid tumor (e.g., a breast cancer, head and neck squamous cell carcinoma, adenoid cystic carcinoma, bladder cancer, pancreatic cancer, hepatocellular carcinoma, melanoma, merkel cell carcinoma, or a colorectal carcinoma). In some embodiment, the solid tumor is accessible for intratumoral administration. In certain embodiment, the cancer is a carcinoma. In certain embodiment, the cancer is a sarcoma (e.g., soft tissue sarcoma). In certain embodiments, the cancer is a hematologic cancer (e.g., a lymphoma).

“Pharmaceutically acceptable carrier” refers to a substance that aids the administration of an active agent to and absorption by a subject and can be included in the compositions described herein without causing a significant adverse toxicological effect on the patient. Non-limiting examples of pharmaceutically acceptable excipients include water, NaCl, normal saline solutions, Phosphate-buffered solution, MgCl₂, KCl, CaCl₂, lactated Ringer’s, normal sucrose, normal glucose, binders, fillers, disintegrants, lubricants, coatings, sweeteners, flavors, salt solutions (such as Ringer’s solution), alcohols, oils, gelatins, carbohydrates such as lactose, amylase or starch, fatty acid esters, lipids, hydroxymethy cellulose, polyvinyl pyrrolidine, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compositions described herein. One of skill in the art will recognize that other pharmaceutical excipients are useful.

### Therapeutic Methods

In some embodiments, provided herein are methods of treating cancer comprising the administration of a pharmaceutical composition comprising one or more bispecific personalized aptamers provided herein. In certain embodiments, the cancer is breast cancer. In some embodiments, the cancer is colorectal carcinoma. Thus, in certain aspects, provided herein is a method of delivering a bispecific personalized aptamer and/or pharmaceutical composition described herein to a subject.

In certain embodiments, the pharmaceutical compositions and aptamers described herein can be administered as monotherapy or in conjunction with any other conventional anti-cancer treatment, such as, for example, radiation therapy and surgical resection of the tumor. These treatments may be applied as necessary and/or as indicated and may occur before, concurrent with or after administration of the pharmaceutical compositions, dosage forms, and kits described herein.

In certain embodiments, the method comprises the administration of multiple doses of the aptamer. Separate administrations can include any number of two or more administrations (e.g., doses), including 2, 3, 4, 5, 6, 7, 8, 9, 10, 11, 12, 13, 14, 15, 16, 20, 21, 22, 23, 24, or 25 administrations. In some embodiments, at least 8, 9, 10, 11, 12, 13, 14, or 15 administrations are included. One skilled in the art can readily determine the number of administrations to perform, or the desirability of performing one or more additional administrations, according to methods known in the art for monitoring therapeutic methods and other monitoring methods provided herein. Accordingly, the methods provided herein include methods of providing to the subject one or more administrations of a bispecific personalized aptamer, where the number of administrations can be determined by monitoring the subject, and, based on the results of the monitoring, determining whether or not to provide one or more additional administrations. Deciding on whether or not to provide one or more additional administrations can be based on a variety of monitoring results, including, but not limited to, indication of tumor growth or inhibition of tumor growth, appearance of new metastases or inhibition of metastasis, the subject’s anti-aptamer antibody titer, the subject’s anti-tumor antibody titer, the overall health of the subject and/or the weight of the subject.

The time period between administrations can be any of a variety of time periods. In some embodiments, the doses may be separated by at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days or 1, 2, 3, or 4 weeks. The time period between administrations can be a function of any of a variety of factors, including acceptable regimen for intratumoral administration, monitoring steps, as described in relation to the number of administrations, the time period for a subject to mount an immune response and/or the time period for a subject to clear the bispecific personalized aptamer. In one example, the time period can be a function of the time period for a subject to mount an immune response; for example, the time period can be more than the time period for a subject to mount an immune response, such as more than about one week, more than about ten days, more than about two weeks, or more than about a month; in another example, the time period can be less than the time period for a subject to mount an immune response, such as less than about one week, less than about ten days, less than about two weeks, or less than about a month. In another example, the time period can be a function of the time period for a subject to clear the bispecific personalized aptamer; for example, the time period can be more than the time period for a subject to clear the bispecific personalized aptamer, such as more than about a day, more than about two days, more than about three days, more than about five days, or more than about a week.

The administered dose of a bispecific personalized aptamer described herein is the amount of the bispecific personalized aptamer that is effective to achieve the desired therapeutic response for a particular patient, composition, and mode of administration, with the least toxicity to the patient or the maximal feasible dose. The effective dosage level can be identified using the methods described herein and will depend upon a variety of factors including the activity of the particular compositions administered (i.e. the potency of the personalized selected arm , the distribution and expression level of the personalized aptamer’s target), the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, the size of the injected target lesion for intratumoral administration, and like factors well known in the medical arts. In general, an effective dose of a cancer therapy will be the amount of the therapeutic agent which is the lowest dose effective to produce a therapeutic effect. Such an effective dose will generally depend upon the factors described above. In some embodiments, about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 71, 7.2, 7.3, 7.4, 7.5, 7.6, 7.7, 7.8, 7.9, 8.0 mg/kg; or 50, 55, 60, 65, 70, 75, 80, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 120, 125, 130, 135, 140, 145, 150, 155, 160, 165, 170, 175, 180, 185, 190, 195, 200 total mg of aptamer or pharmaceutical composition is administered (e.g., intratumorally administered) per dose.

Examples of routes of administration include oral administration, rectal administration, topical administration, inhalation (nasal) or injection. Administration by injection includes intravenous (IV), intratumoral, peritumoral, intramuscular (IM), and subcutaneous (SC) administration. The compositions described herein can be administered in any form by any effective route, including but not limited to oral, parenteral, enteral, intravenous, intratumoral, intravesical, intraperitoneal, topical, transdermal (e.g., using any standard patch), intradermal, ophthalmic, (intra)nasally, local, non-oral, such as aerosol, inhalation, subcutaneous, intramuscular, buccal, sublingual, (trans)rectal, vaginal, intraarterial, and intrathecal, transmucosal (e.g., sublingual, lingual, (trans)buccal, (trans)urethral, vaginal (e.g., trans- and perivaginally), implanted, intrapulmonary, intraduodenal, intragastrical, and intrabronchial. In some embodiments, the bispecific personalized aptamers described herein are administered orally, rectally, topically, intravesically, by injection into or adjacent to a draining lymph node, intravenously, by inhalation or aerosol, or subcutaneously. In some embodiments, the administration is parenteral administration (e.g., subcutaneous administration). The administration may be an intratumoral injection or a peritumoral injection.

The dosage regimen can be any of a variety of methods and amounts, and can be determined by one skilled in the art according to known clinical factors. As is known in the medical arts, dosages for any one patient can depend on many factors, including the subject’s species, size, body surface area, age, sex, immunocompetence, tumor dimensions general health and specific biomarkers, the particular bispecific personalized aptamer to be administered, duration and route of administration, the kind and stage of the disease, for example, tumor size, and other compounds such as drugs being administered concurrently.

The methods of treatment described herein may be suitable for the treatment of a primary tumor, a secondary tumor or metastasis, as well as for recurring tumors or cancers. The dose of the pharmaceutical compositions described herein may be appropriately set or adjusted in accordance with the dosage form, the route of administration, the degree or stage of a target disease, and the like.

In some embodiments, the dose administered to a subject is sufficient to prevent cancer, delay its onset, or slow or stop its progression or prevent a relapse of a cancer, reduce tumor burden, or contribute to the disease -free survival, time to progression or overall survival of the subject. One skilled in the art will recognize that dosage will depend upon a variety of factors including the strength of the particular compound employed, as well as the age, species, condition, and body weight of the subject. The size of the dose will also be determined by the route, timing, and frequency of administration as well as the existence, nature, and extent of any adverse side-effects that might accompany the administration of a particular compound and the desired physiological effect.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. An effective dosage and treatment protocol can be determined by routine and conventional means, starting e.g., with a low dose in laboratory animals and then increasing the dosage while monitoring the effects, and systematically varying the dosage regimen as well. Animal studies are commonly used to determine the maximal tolerable dose (“MTD”) of bioactive agent per kilogram weight. Those skilled in the art regularly extrapolate doses for efficacy, while avoiding toxicity, in other species, including humans.

In accordance with the above, in therapeutic applications, the dosages of the aptamers provided herein may vary depending on the specific aptamer, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and most preferably causing complete regression of the cancer.

Examples of cancers that may treated by methods described herein include, but are not limited to, hematological malignancy, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross’ leukemia, Rieder cell leukemia, Schilling’s leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher’s carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, chondrosarcoma, fibrosarcoma, lymphosarcoma, melanosarcoma, myxosarcoma, osteosarcoma, endometrial sarcoma, stromal sarcoma, Ewing’s sarcoma, fascial sarcoma, fibroblastic sarcoma, giant cell sarcoma, Abemethy’s sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms’ tumor sarcoma, granulocytic sarcoma, Hodgkin’s sarcoma, idiopathic multiple pigmented hemorrhagic sarcoma, immunoblastic sarcoma of B cells, lymphoma, immunoblastic sarcoma of T-cells, Jensen’s sarcoma, Kaposi’s sarcoma, Kupffer cell sarcoma, angiosarcoma, leukosarcoma, malignant mesenchymoma sarcoma, parosteal sarcoma, reticulocytic sarcoma, rhabdosarcoma, serocystic sarcoma, synovial sarcoma, telangiectaltic sarcoma, Hodgkin’s Disease, Non-Hodgkin’s Lymphoma, multiple myeloma, neuroblastoma, bladder cancer, breast cancer, ovarian cancer, lung cancer, colorectal cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, Harding-Passey melanoma, juvenile melanoma, lentigo maligna melanoma, malignant melanoma, acral-lentiginous melanoma, amelanotic melanoma, benign juvenile melanoma, Cloudman’s melanoma, S91 melanoma, nodular melanoma subungal melanoma, superficial spreading melanoma, plasmacytoma, colorectal cancer, rectal cancer.

In some embodiments, the methods and compositions provided herein relate to the treatment of a sarcoma. The term “sarcoma” generally refers to a tumor which is made up of a substance like the embryonic connective tissue and is generally composed of closely packed cells embedded in a fibrillar, heterogeneous, or homogeneous substance. Sarcomas include, but are not limited to, chondrosarcoma, fibrosarcoma, lymphosarcoma, melanosarcoma, myxosarcoma, osteosarcoma, endometrial sarcoma, stromal sarcoma, Ewing’s sarcoma, fascial sarcoma, fibroblastic sarcoma, giant cell sarcoma, Abemethy’s sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms’ tumor sarcoma, granulocytic sarcoma, Hodgkin’s sarcoma, idiopathic multiple pigmented hemorrhagic sarcoma, immunoblastic sarcoma of B cells, lymphoma, immunoblastic sarcoma of T-cells, Jensen’s sarcoma, Kaposi’s sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Additional exemplary neoplasias that can be treated using the methods and compositions described herein include Hodgkin’s Disease, Non-Hodgkin’s Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

In some embodiments, the cancer treated is a melanoma. The term “melanoma” is taken to mean a tumor arising from the melanocytic system of the skin and other organs. Non-limiting examples of melanomas are Harding-Passey melanoma, juvenile melanoma, lentigo maligna melanoma, malignant melanoma, acral-lentiginous melanoma, amelanotic melanoma, benign juvenile melanoma, Cloudman’s melanoma, S91 melanoma, nodular melanoma subungal melanoma, and superficial spreading melanoma.

Particular categories of tumors that can be treated using methods and compositions described herein include lymphoproliferative disorders, breast cancer, ovarian cancer, prostate cancer, cervical cancer, endometrial cancer, bone cancer, liver cancer, stomach cancer, colon cancer, colorectal cancer, pancreatic cancer, cancer of the thyroid, head and neck cancer, cancer of the central nervous system, cancer of the peripheral nervous system, skin cancer, kidney cancer, as well as metastases of all the above. Particular types of tumors include hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing’s tumor, leimyosarcoma, rhabdotheliosarcoma, invasive ductal carcinoma, papillary adenocarcinoma, melanoma, pulmonary squamous cell carcinoma, basal cell carcinoma, adenocarcinoma (well differentiated, moderately differentiated, poorly differentiated or undifferentiated), bronchioloalveolar carcinoma, renal cell carcinoma, hypernephroma, hypernephroid adenocarcinoma, bile duct carcinoma, choriocarcinoma, seminoma, embryonal carcinoma, Wilms’ tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin’s lymphoma, non-Hodgkin’s lymphoma.

Cancers treated in certain embodiments also include precancerous lesions, e.g., actinic keratosis (solar keratosis), moles (dysplastic nevi), acitinic chelitis (farmer’s lip), cutaneous horns, Barrett’s esophagus, atrophic gastritis, dyskeratosis congenita, sideropenic dysphagia, lichen planus, oral submucous fibrosis, actinic (solar) elastosis and cervical dysplasia.

Cancers treated in some embodiments include non-cancerous or benign tumors, e.g., of endodermal, ectodermal or mesenchymal origin, including, but not limited to cholangioma, colonic polyp, adenoma, papilloma, cystadenoma, liver cell adenoma, hydatidiform mole, renal tubular adenoma, squamous cell papilloma, gastric polyp, hemangioma, osteoma, chondroma, lipoma, fibroma, lymphangioma, leiomyoma, rhabdomyoma, astrocytoma, nevus, meningioma, and ganglioneuroma.

In certain embodiments, the cancer is a solid tumor (e.g., breast cancer, head and neck squamous cell carcinoma, adenoid cystic carcinoma, bladder cancer, pancreatic cancer, hepatocellular carcinoma, melanoma, merkel cell carcinoma, or a colorectal carcinoma). In some embodiment, the solid tumor is accessible for intratumoral administration. In certain embodiment, the cancer is a sarcoma (e.g., soft tissue sarcoma). In certain embodiments, the cancer is a hematologic cancer (e.g., a lymphoma).

### Methods of Identifying the Cancer Cell-targeting Strand

In some embodiments, the cancer cell-binding strand is identified via a selex process. In certain embodiments, multiple rounds (e.g., 3 rounds) of binding selex is performed using targeted cancer cells to identify aptamers than bind to the cancer cell target. In certain embodiments, a functional selex assay is also performed via a process comprising: (a) contacting a cancer cell with a plurality of particles on which are immobilized a library of aptamer clusters (“aptamer cluster particles”), wherein at least a subset of the immobilized aptamer clusters bind to at least a subset of the cancer cell to form cell-aptamer cluster particle complexes; (b) incubating the cell-aptamer cluster particle complexes for a period of time sufficient for at least some of the cancer cell in the cell-aptamer cluster particle complexes to undergo cell function; (c) detecting the cell-aptamer cluster particle complexes undergoing the cell function; (d) separating cell-aptamer cluster particle complexes comprising cancer cell undergoing the cell function detected in step (c) from other cell-aptamer cluster particle complexes; (e) amplifying the aptamers in the separated cell-aptamer cluster particle complexes to generate a functionally enriched population of aptamers; and (f) identifying the enriched population of aptamers via sequencing, thereby identifying the cancer cell-binding strand. In specific embodiments, a reporter of cell death is added after the incubation of the cancer cell with the aptamer cluster particles, but prior to the detection of the cell-aptamer cluster particle complexes that undergo the cell function.

In some embodiments, steps (c) and (d) are performed using a flow cytometer. In some embodiments, the methods described herein further comprise separating the aptamer cluster particles from the target cells in the cell-aptamer cluster particle complexes separated in step (d) via heat denaturation. In some embodiments, the methods described herein further comprise the step of dissociating the aptamers from the particles in the separated aptamer cluster particles. In some embodiments, the methods described herein further comprise a step (e′) after step (e) and before step (f): (i) forming aptamer cluster particles from the functionally enriched population of aptamers of step (e); and (ii) repeating steps (a) - (e) using the newly formed aptamer cluster particles to generate a further functionally enriched population of aptamers.

In certain embodiments, the step of enriching the population of functional aptamers involves applying a restrictive condition (e.g., reducing the total number of particles) in the successive rounds. In some embodiments, the population of aptamers of each additional round of screening is functionally enriched by a factor of at least 1.1 (e.g., by a factor of about 1.1, 1.2, 1.3, 1.4, 1.5, 1.6, 1.7. 1.8, 1.9, 2.0, 2.1, 2.2, 2.3, 2.4, or 2.5). The number of rounds of enrichment can be as many as desired. For example, in some embodiments, the number of rounds are at least 2 (e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100).

The library of aptamer cluster particles can be incubated with cancer cells under any condition conductive to form cell-aptamer cluster particle complexes and to allow the aptamer cluster particles to provide an effect on the cancer cells. The condition includes, but is not limited to, for examples, a controlled period of time, an optimal temperature (e.g., 37° C.), and/or an incubating medium (e.g., cancer cell culture medium), etc. The period of time of incubation can be from about 10 minutes to about 5 days, from about 30 minutes to about 4 days, from about 1 hour to about 3 days, from about 1.5 hours to about 24 hours, or from about 1.5 hours to about 2 hours. In some embodiments, the period of time of incubation may be, for example, 10 min, 15 min, 30 min, 45 min, 1 hour, 2 hours, 4 hours, 6 hours, 8 hours, 12 hours, 16 hours, 24 hours, 2 days, 3 days, 4 days, 5 days, 6 days, or 7 days.

The cancer cells and the aptamer cluster particles may be mixed at a ratio from 10:1 to 1 :2000 (e.g., at a ratio of 10:1, 5:1, 1:1, 1:5, 1:10, 1:15, 1:20, 1:25, 1:30, 1:33, 1:35, 1:40, 1:45, 1:50, 1:55, 1:60, 1:65, 1:70, 1:75, 1:80, 1:85, 1:90, 1:95, 1:100, 1:150, 1:200, 1:250, 1:300, 1:350, 1:400, 1:450, 1:500, 1:550, 1:600, 1:650, 1:700, 1:750, 1:800, 1:850, 1:900, 1:950, 1:1000, 1:1100, 1:1200, 1:1300, 1:1400, 1:1500, 1:1600, 1:1700, 1:1800, 1:1900, 1:2000). The formed cell-aptamer cluster particle complexes may comprise about 1 to 50 particles per cancer cell (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 particles per cancer cell). In certain embodiment, the formed cell-aptamer cluster particle complexes comprise about 2 to 10 particles per cancer cell. In some embodiments, the aptamer cluster particle in the formed cell-aptamer cluster particle complexes comprises about 1 to 10 clusters per particle (e.g., 1, 2, 3, 4, 5, 6, 7, 8, 9, or 10 clusters per particle). In certain embodiments, the aptamer cluster particle in the formed cell-aptamer cluster particle complexes comprises about 1 to 6 clusters per particle.

In some embodiments, the cancer cells are labeled with and/or comprises a detectable label. The cancer cells can be detectably labeled directly (e.g., through a direct chemical linker) or indirectly (e.g., using a detectably labeled cancer cell-specific antibody). In some embodiments, cancer cells can be labeled by incubating the cancer cell with the detectable label under conditions such that the detectable label is internalized by the cell. In some embodiments, the cancer cell is detectably labeled before performing the aptamer screening methods described herein. In some embodiments, the cancer cell is labeled during the performance of the aptamer screening methods provided herein. In some embodiments, the cancer cell is labeled after it is bound to an aptamer cluster (e.g., by contacting the bound target with a detectably labeled antibody). In some embodiments, any detectable label can be used. Examples of detectable labels include, but are not limited to, fluorescent moieties, radioactive moieties, paramagnetic moieties, luminescent moieties and/or colorimetric moieties. In some embodiments, the cancer cells described herein are linked to, comprise and/or are bound by a fluorescent moiety. Examples of fluorescent moieties include, but are not limited to, Allophycocyanin (APC), Fluorescein, Fluorescein isothiocyanate (FITC), Phycoerythrin (PE), Cy3 dye, Cy5 dye, Peridinin-chlorophyll protein complex, Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 568, Alexa Fluor 594, Alexa Fluor 633, Alexa Fluor 635, Alexa Fluor 647, Alexa Fluor 660, Alexa Fluor 680, Alexa Fluor 700, Alexa Fluor 750, Alexa Fluor 790, EGFP, mPlum, mCherry, mOrange, mKO, EYFP, mCitrine, Venus, YPet, Emerald, Cerulean and CyPet.

In some embodiments, the cancer cell contacted with the aptamer cluster particles is live/viable. In other embodiments, the cancer cell contacted with the aptamer cluster particles is fixed or in suspension.

In some embodiments, the cancer cell is a human cancer cell or a patient-derived cancer cell. In some embodiments, the cell is from any cancerous or pre-cancerous tumor. Non-limiting examples of cancer cells include cancer cells from the bladder, blood, bone, bone marrow, brain, breast, colon, esophagus, gastrointestine, gum, head, kidney, liver, lymph nodes, lung, nasopharynx, neck, ovary, pancreas, prostate, skin, stomach, testis, tongue, salivary glands or uterus. In addition, the cancer may specifically be of the following histological type, though it is not limited to these: neoplasm, malignant, carcinoma, carcinoma, undifferentiated, giant and spindle cell carcinoma, small cell carcinoma, papillary carcinoma, squamous cell carcinoma, lymphoepithelial carcinoma, basal cell carcinoma, pilomatrix carcinoma, transitional cell carcinoma, papillary transitional cell carcinoma, adenocarcinoma, gastrinoma, malignant, cholangiocarcinoma, hepatocellular carcinoma, combined hepatocellular carcinoma and cholangiocarcinoma, trabecular adenocarcinoma, adenoid cystic carcinoma, adenocarcinoma in adenomatous polyp, adenocarcinoma, familial polyposis coli, solid carcinoma, carcinoid tumor, malignant, branchiolo-alveolar adenocarcinoma, papillary adenocarcinoma, chromophobe carcinoma, acidophil carcinoma, oxyphilic adenocarcinoma, basophil carcinoma, clear cell adenocarcinoma, granular cell carcinoma, follicular adenocarcinoma, papillary and follicular adenocarcinoma, nonencapsulating sclerosing carcinoma, adrenal cortical carcinoma, endometroid carcinoma, skin appendage carcinoma, apocrine adenocarcinoma, sebaceous adenocarcinoma, ceruminous adenocarcinoma, mucoepidermoid carcinoma, cystadenocarcinoma, papillary cystadenocarcinoma, papillary serous cystadenocarcinoma, mucinous cystadenocarcinoma, mucinous adenocarcinoma, signet ring cell carcinoma, infiltrating duct carcinoma, medullary carcinoma, lobular carcinoma, inflammatory carcinoma, paget’s disease, mammary, acinar cell carcinoma, adenosquamous carcinoma, adenocarcinoma w/squamous metaplasia, thymoma, malignant, ovarian stromal tumor, malignant, thecoma, malignant, granulosa cell tumor, malignant, and roblastoma, malignant, sertoli cell carcinoma, leydig cell tumor, malignant, lipid cell tumor, malignant, paraganglioma, malignant, extra-mammary paraganglioma, malignant, pheochromocytoma, glomangiosarcoma, malignant melanoma, amelanotic melanoma, superficial spreading melanoma, malig melanoma in giant pigmented nevus, epithelioid cell melanoma, blue nevus, malignant, sarcoma, fibrosarcoma, fibrous histiocytoma, malignant, myxosarcoma, liposarcoma, leiomyosarcoma, rhabdomyosarcoma, embryonal rhabdomyosarcoma, alveolar rhabdomyosarcoma, stromal sarcoma, mixed tumor, malignant, mullerian mixed tumor, nephroblastoma, hepatoblastoma, carcinosarcoma, mesenchymoma, malignant, brenner tumor, malignant, phyllodes tumor, malignant, synovial sarcoma, mesothelioma, malignant, dysgerminoma, embryonal carcinoma, teratoma, malignant, struma ovarii, malignant, choriocarcinoma, mesonephroma, malignant, hemangiosarcoma, hemangioendothelioma, malignant, kaposi’s sarcoma, hemangiopericytoma, malignant, lymphangiosarcoma, osteosarcoma, juxtacortical osteosarcoma, chondrosarcoma, chondroblastoma, malignant, mesenchymal chondrosarcoma, giant cell tumor of bone, ewing’s sarcoma, odontogenic tumor, malignant, ameloblastic odontosarcoma, ameloblastoma, malignant, ameloblastic fibrosarcoma, pinealoma, malignant, chordoma, glioma, malignant, ependymoma, astrocytoma, protoplasmic astrocytoma, fibrillary astrocytoma, astroblastoma, glioblastoma, oligodendroglioma, oligodendroblastoma, primitive neuroectodermal, cerebellar sarcoma, soft tissue sarcoma, ganglioneuroblastoma, neuroblastoma, retinoblastoma, olfactory neurogenic tumor, meningioma, malignant, neurofibrosarcoma, neurilemmoma, malignant, granular cell tumor, malignant, malignant lymphoma, Hodgkin’s disease, Hodgkin’s lymphoma, paragranuloma, malignant lymphoma, small lymphocytic, malignant lymphoma, large cell, diffuse, malignant lymphoma, follicular, mycosis fungoides, other specified non-Hodgkin’s lymphomas, malignant histiocytosis, multiple myeloma, mast cell sarcoma, immunoproliferative small intestinal disease, leukemia, lymphoid leukemia, plasma cell leukemia, erythroleukemia, lymphosarcoma cell leukemia, myeloid leukemia, basophilic leukemia, eosinophilic leukemia, monocytic leukemia, mast cell leukemia, megakaryoblastic leukemia, myeloid sarcoma, and hairy cell leukemia.

In some embodiments, the detectable label is a fluorescent dye. Non-limiting examples of fluorescent dyes include, but are not limited to, a calcium sensitive dye, a cell tracer dye, a lipophilic dye, a cell proliferation dye, a cell cycle dye, a metabolite sensitive dye, a pH sensitive dye, a membrane potential sensitive dye, a mitochondrial membrane potential sensitive dye, and a redox potential dye. In certain embodiment, the cancer cell is labeled with an activation associated marker, an oxidative stress reporter, an angiogenesis marker, an apoptosis marker, an autophagy marker, an immunological cell death marker a cell viability marker, or a marker for ion concentrations.

In some embodiments, the cancer cell is labeled prior to exposure of aptamers to the cancer cell. In some embodiments, the cancer cell is labeled after exposure of aptamers to the cancer cell. In one embodiment, the cancer cell is labeled with fluorescently-labeled antibodies, antibody fragments and artificial antibody-based constructs, fusion proteins, sugars, or lectins. In another embodiment, the cancer cell is labeled with fluorescently-labeled antibodies, antibody fragments and artificial antibody-based constructs, fusion proteins, sugars, or lectins after exposure of aptamers to the cancer cell.

In certain embodiments, the cellular function is cell death. Exemplary cell death reporters include but not limited to ones directed at cleaved/ activated caspase-3,7, 8 or 9, annexin V, Mitochondrial Membrane Potential, calreticulin, heat-shock proteins, ATP and HMGB1.

TABLE 3

Exemplary probes

| | | |
|---|---|---|
| Probe Name | Distributer | CAT# |
| CellEvent Caspase-3/7 | Invitrogen | C10423 |
| MitoProbe Dilc1(5) | Invitrogen | M34151 |
| Annexin V | BioLegend | 640945 |
| Violet Ratiometric Membrane Asymmetry | Invitrogen | A35137 |
| Violet Live Cells Caspase | BD Pharmigen | 565521 |
| Caspase-8 (active) | abcam | ab65614 |
| Caspase-9 (active) | abcam | ab65615 |
| MitoProbe DiOC₂(3) | Invitrogen | M34150 |
| CellTrace Calcein Violet | Invitrogen | C34 858 |

In some embodiments, the reporter of cellular function is an antibody. In certain embodiments, the antibody is labeled with a fluorescent moiety. Examples of fluorescent moieties include, but are not limited to Allophycocyanin (APC), Fluorescein, Fluorescein isothiocyanate (FITC), Phycoerythrin (PE), Cy3 dye, Cy5 dye, Peridinin-chlorophyll protein complex, Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 568, Alexa Fluor 594, Alexa Fluor 633, Alexa Fluor 635, Alexa Fluor 647, Alexa Fluor 660, Alexa Fluor 680, Alexa Fluor 700, Alexa Fluor 750, Alexa Fluor 790, EGFP, mPlum, mCherry, mOrange, mKO, EYFP, mCitrine, Venus, YPet, Emerald, Cerulean and CyPet.

In some embodiments, the cellular function is cell proliferation and the antibody binds to a proliferation marker (e.g., Ki67, MCM2, PCNA).

In some embodiments, the cellular function is tumor antigen expression and the antibody binds to a tumor antigen (e.g., Prostate-specific antigen (PSA), Prostate Membrane Antigen (PSMA)Cancer antigen 15-3 (CA-15-3), Carcinoembryonic antigen (CEA), Cancer antigen 125 (CA-125), Alpha-fetoprotein (AFP), NY-ESO-1, MAGEA-A3, WT1, hTERT, Tyrosinase, gp100, MART-1, melanA, B catenin, CDC27, HSP70-2-m, HLA-A2-R17OJ, AFP, EBV-EBNA, HPV16-E7, MUC-1, HER-2/neu, Mammaglobin-A).

In some embodiments, the library can be, for example, newly synthesized, or an output of a previous selection process. This process can involve one or more positive selection cycles, one or more negative selection cycles, or both, in any combination and sequence.

The prepared library is mounted on particles, such as beads. Emulsion PCR (ePCR) amplification turns each single sequence from the initial library into a cluster of at least, e.g., 10,000 copies of the same sequence. The library of aptamer cluster particles are then incubated with cancer cells. The cancer cells can be labeled prior to introduction into the aptamer cluster particles with a fluorescent dye, for the purpose of reporting a biological or chemical effect on the cancer cells. The cancer cells and the library of aptamer cluster particles are incubated for a certain amount of time to allow the effect to take place. Fluorescent dyes or markers for reporting the biological or chemical effect (e.g., cell apoptosis, etc.) can then be added to the cancer cells. In some embodiments, the reporter is added to the cells before the incubation. In some embodiments, the reporter is added during the incubation. In certain embodiments the reporter is added after incubation. In some embodiments a second reporter is used (e.g., before incubation) to mark cells expressing the wanted phenotype (e.g. apoptosis) with no relation to the incubation process with the aptamers. In certain embodiments, the second reporter helps distinguish false positives. In some embodiments a second (or third) reporter is used (e.g., a reporter that works via a different mechanism) in order to make sure the phenotype detected is not false positive. Effect-positive clusters are then sorted away from the effect-negative clusters and corresponding functional aptamer sequences are analyzed. The sorted positive clusters can also be amplified and immobilized to the surface of particles as the initial library for additional rounds of screening. A portion of the enriched functional aptamers after each round of screening is subjected to output sampling and comparative functional analysis before the identification of the aptamers by sequencing.

Additional methods for generating aptamer libraries and immobilized aptamer clusters, as well as methods of identifying aptamers that specifically modulate a target cell function (e.g., aptamers that induce cancer cell apoptosis) by screening an aptamer library have been described in the PCT Application No: PCT/IB2019/001082, which is incorporated herein by reference.

The immune effector cell-binding strand may be identified using methods which are known to the skilled person. For example, the immune effector cell-binding strand may be identified using Cell-SELEX binding process described in the examples and figures of the present disclosure. The immune effector cell-binding strand may also be identified from the literature.

### Methods of Making Bispecific Personalized Aptamers

In certain aspects, provided herein is a method of making a bispecific personalized aptamer. In some embodiments, the method comprises (1) synthesizing a cancer cell-binding strand; (2) synthesizing an immune effector cell-binding strand; (3) linking both strands to form the bispecific personalized aptamer. The two strands may be linked via complementary sequences hybridization, a covalent bond, or a PEG bridge.

After identifying the cancer cell-binding strand and the immune effector cell-binding strand, both strands may be synthesized by methods which are well known to the skilled person. For example, synthesis of different aptamers may be performed by the well-established automated solid phase phosphoramidite chemistry. As per the programmed sequence, one nucleotide is added per synthesis cycle, which consists of a series of steps.

Briefly, the synthesis cycle starts with the removal of the acid-labile 5′-dimethoxytrityl protection group (DMT, “Trityl”) from the hydroxyl function of the terminal, support-bound nucleoside by UV-controlled treatment with an organic acid. The exposed highly-reactive hydroxyl group is now available to react in the coupling step with the next protected nucleoside phosphoramidite building block, forming a phosphite triester backbone. Next, the acid-labile phosphite triester backbone is oxidized to the stable pentavalent phosphate trimester. If a phosphorothioate modification is desired at a specific backbone position, the acid labile phosphite trimester backbone is sulfuridized at this step, instead of the oxidation process, to generate a P=S bond rather than a P=O. Successively, all the unreacted 5′-hydroxyl groups are acetylated (“capped”) in order to block these sites during the next coupling step, avoiding internal mismatch sequences. Following the capping step, the cycle starts again by removal of the DMT-protection group and successive coupling of the next base according to the desired sequence. Finally, the oligonucleotide is cleaved from the solid support and all protection groups are removed from the backbone and bases.

In some embodiments, the synthesized cancer cell-binding strand and the synthesized immune effector cell-binding strand further comprise complementary 5′ sequences. In some embodiments, the synthesized cancer cell-binding strand and the synthesized immune effector cell-binding strand further comprise complementary 3′ sequences. In some embodiments, the step (3), i.e., linking both strands to form the bispecific personalized aptamer, comprises hybridizing the synthesized cancer cell-binding strand and the synthesized immune effector cell-binding strand. In some embodiments, the complementary 5′ or 3′ sequence comprising one or more CpG-motifs. In preferred embodiments, the complementary 5′ or 3′ sequences of the synthesized cancer cell-binding strand and the synthesized immune effector cell-binding strand are hybridized to form a double-stranded CpG-rich sequence.

In some embodiments, the complementary 5′ sequence comprises a nucleic acid sequence that is at least 60% identical (e.g., at least 65% identical, at least 70% identical, at least 75% identical, at least 80% identical, at least 85% identical, at least 90% identical, at least 92% identical, at least 94% identical, at least 96% identical, at least 98% identical) to any one of SEQ ID NOs: 63-66. In some embodiments, the complementary 5′ sequence comprises a nucleic acid sequence of any one of SEQ ID NOs: 63-66. In certain embodiments, the complementary 5′ sequence comprises a nucleic acid sequence that comprises at least 12 (e.g., at least 13, at least 14, at least 15, at least 16, at least 17, at least 18, at least 19, at least 20, at least 21, at least 22) consecutive nucleotides of any one of SEQ ID NO: 63-66. In some embodiments, the complementary 5′ sequence has a sequence consisting essentially of SEQ ID NOs: 63-66. In certain embodiments, the complementary 5′ sequence has a sequence consisting of SEQ ID NO: 63-66.

In some embodiments, the method comprises synthesizing (e.g., chemically synthesizing) a cancer cell-binding strand comprising a nucleic acid sequence that is at least 60% identical (e.g., at least 65% identical, at least 70% identical, at least 75% identical, at least 80% identical, at least 85% identical, at least 90% identical, at least 92% identical, at least 94% identical, at least 96% identical, at least 98% identical) to any one of SEQ ID NOs: 43-62 or 107-115. In some embodiments, the method comprises synthesizing a cancer cell-binding strand comprising a nucleic acid sequence of any one of SEQ ID NOs: 43-62 or 107-115. In certain embodiments, the method comprises synthesizing a cancer cell-binding strand comprising a nucleic acid sequence that comprises at least 30 (e.g., at least 35, at least 40, at least 45, at least 50, at least 55, at least 60) consecutive nucleotides of any one of SEQ ID NO: 43-62 or 107-115. In some embodiments, the method comprises synthesizing a cancer cell-binding strand having a sequence consisting essentially of SEQ ID NOs: 43-62 or 107-115. In certain embodiments, the method comprises a cancer cell-binding strand having a sequence consisting of SEQ ID NO: 43-62 or 107-115.

In some embodiments, the method comprises synthesizing (e.g., chemically synthesizing) an immune effector cell-binding strand comprising a nucleic acid sequence that is at least 60% identical (e.g., at least 65% identical, at least 70% identical, at least 75% identical, at least 80% identical, at least 85% identical, at least 90% identical, at least 92% identical, at least 94% identical, at least 96% identical, at least 98% identical) to any one of SEQ ID NOs: 1-42, 88-106 or 116. In some embodiments, the method comprises synthesizing an immune effector cell-binding strand comprising a nucleic acid sequence of any one of SEQ ID NOs: 1-42, 88-106 or 116. In certain embodiments, the method comprises synthesizing an immune effector cell-binding strand comprising a nucleic acid sequence that comprises at least at least 20 (e.g., at least 25, at least 30, at least 35, at least 40, at least 45, at least 50) consecutive nucleotides of any one of SEQ ID NO: 1-42, 88-106 or 116. In some embodiments, the method comprises synthesizing an immune effector cell-binding strand having a sequence consisting essentially of SEQ ID NOs: 1-42, 88-106 or 116. In certain embodiments, the method comprises synthesizing a nucleic acid having a sequence consisting of SEQ ID NOs: 1-42, 88-106 or 116.

### EXAMPLES

### Example 1- Bispecific Personalized Aptamers

### A. Representative Structures of Bispecific Personalized Aptamers

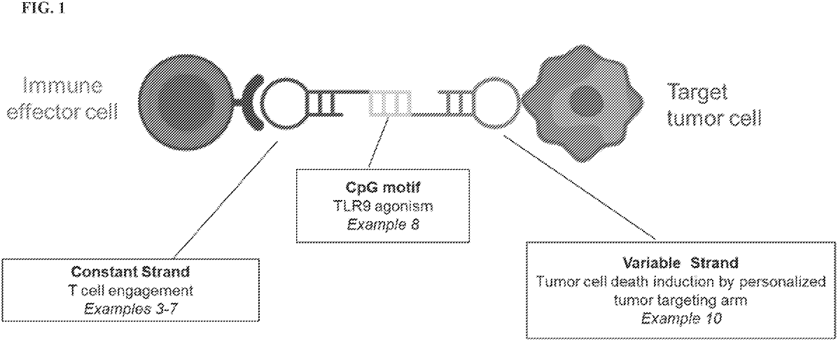

In some aspects, personalized cancer therapeutics described herein are composed of a heterodimeric structure with three separate domains (FIG. 1).

In certain aspects, the platform described herein is designed to yield patient-tailored cancer therapeutics to treat patients with individualized solutions optimized for the unique set of conditions and potential drug targets presented by each patient as reflected by fresh sample tissues of their tumors. In some embodiments, bispecific personalized aptamers are designed to target specific neoantigens and surface molecules displayed by cancer cells of patients and to facilitate both direct lethality of cancer cells as well as immune-associated responses. In some embodiments, efficacy is achieved through three separate modes-of-actions (MoAs) incorporated into a single therapeutic entity, as described below:

### 1. Personalized Strand: Direct Killing of Cancer Cells by Personalized Aptamer

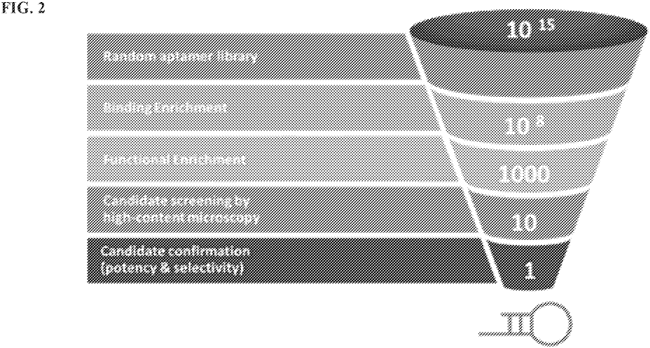

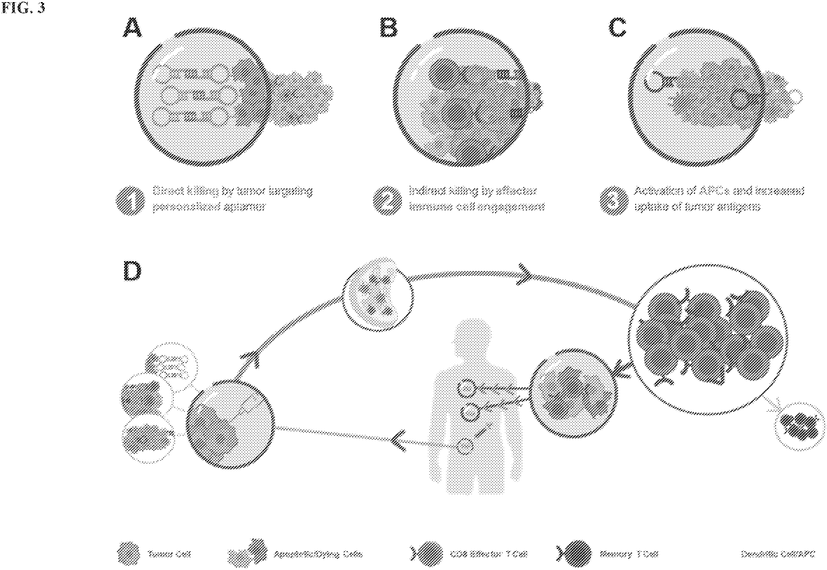

In some embodiments, this moiety is selected through a process initiating from a random pool of 10¹⁵ potential leads and is described in detail in the PCT Application No. PCT/IB19/01082. Briefly, the personalized process is designed to identify aptamers that best facilitate targeted killing of cancer cells while not harming healthy cells. The patient -specific strand is identified by conducting Binding and Functional Enrichment Processes (Cell and Functional SELEX), screening candidates with high-throughput microscopy, and confirming the activity and specificity of top candidates, while including selectivity tests and attempting to rule out off-target effects. (FIG. 2 and FIG. 3A).

### 2. Immune-Modulating Strand: Cancer Cell Lysis Through T or NK Cell-Mediated Cytotoxicity

In some embodiments, this aptamer arm is a CD3 binding aptamer disclosed herein (e.g., comprising a sequence of any one of SEQ ID NO. 88-106 or 116) (FIG. 3B). This immune-modulating arm could potentially be designed to be shared across different patients.

### 3. CpG Motif With TLR9-Agonistic Activity

In some embodiments, the two aptamer arms of the bispecific structure are bridged together by nucleic-base hybridization of single stranded overhangs of complementary sequences. This hybridization domain is CpG rich and designed to induce TLR9-mediated antigen presenting cell (APCs) stimulation and increased uptake of tumor antigens (FIG. 3C). Stimulated APCs would subsequently migrate to the tumor draining lymph nodes and cross-present the engulfed tumor antigens to cytotoxic T lymphocytes, resulting in an adaptive, systemic, anti-tumor immune response (FIG. 3D).

### B. Personalized Process for Each Patient

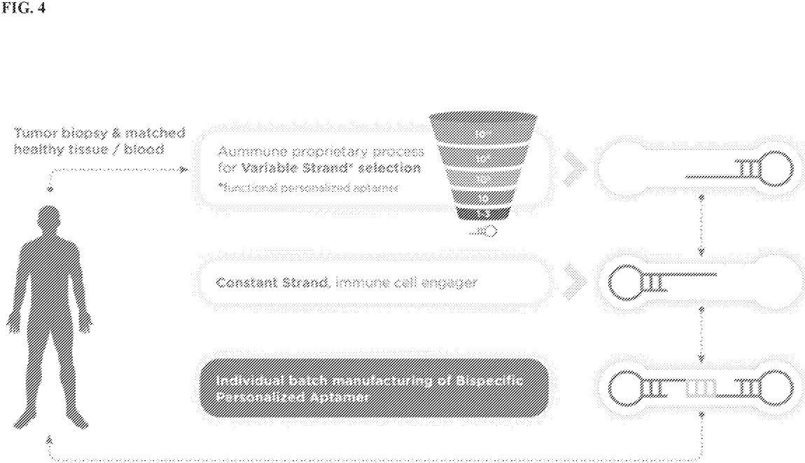

In some embodiments, as a cancer therapeutic platform, the personalized process contains several critical steps (FIG. 4):
1. Receipt of two types of primary matched samples from the subject
   a. Tumor biopsy
   b. Healthy tissue to be used as a negative control which will consist of either normal tissue from the site of biopsy or Peripheral Blood Mononuclear Cells (PBMCs).
2. Implementation of the selection process described herein to identify a personalized aptamer which induces tumor cell death while leaving healthy cell intact;
3. Manufacturing and hybridization of both strands to yield bispecific personalized aptamers;
4. Bispecific personalized aptamer is administered to the respective individual subj ect.

### Example 2 —Materials and Methods for Examples 3-4

### A. Materials

### A. Random Library

Random library 2.6 was purchased from IDT. The library contains a vast repertoire of approximately 10¹⁵ different 50 nt-longrandom sequences flanked by two unique sequences at the 3′ and 5′ acting as primers for PCR amplification during the SELEX procedure. The lyophilized library (“Lib 2.6”) was reconstituted in ultra-pure water (UPW) to a final concentration of 1 mM.

The random library sequence was:

5’TATCCGTCTGCTCTCGCTATNNNNNNNNNNNNNNNNNNNNNNNNNNNNNNN
NNNNNNNNNNNNNNNNNNACGCACCTAATGTCCTACTG-3’ (SEQ ID NO: 71),

where N represents a random oligonucleotide selected from a mixture of equally represented T, A, C and G nucleotides.

### B. Pre-SELEX Preparation

Library 2.6 (Lib 2.6) underwent QC validation using HPLC gel filtration column.

### C. Library Primers and Caps

A set of 20 nt primers and caps were purchased from IDT. Caps were used to hybridize to the Library’s primer sites during incubation with cells in order to refrain from the possibility of primer sequences interacting with the random 50 nt sequence site. A mixture of 3′ and 5′ caps in each SELEX round was used in a 3:1 caps-to-library ratio.

The forward primer was purchased from IDT labelled with Cy-5 at the 5′ site for sequence amplification that was detected in a fluorescence assay. The lyophilized primers were reconstituted in ultra-pure water (UPW) to a final concentration of 100 µM.

TABLE 4

Random library, primers and caps sequences

| Aptamer name | SEQ ID NO: | Sequence 5′ to 3′ |
|---|---|---|
| Random Library | 71 | TATCCGTCTGCTCTCGCTATNNNNNNNNNNNNNNNNNNNNNNNNNNNNNNNNNNNNNN NNNNNNNNNNNACGCACCTAATGTCCTACTG |
| Forward Primer | 72 | TATCCGTCTGCTCTCGCTAT |
| Reverse/3′ cap | 73 | CAGTAGGACATTAGGTGCGT |
| 5′ cap | 74 | ATAGCGAGAGCAGACGGATA |
| Forward labelled Cy-5 | 75 | /5Cy5/TATCCGTCTGCTCTCGCTAT |

### D. Aptamer Folding Buffer

Phosphate-buffered saline (minus Magnesium and Calcium) was supplemented with 1 mM Magnesium Chloride (MgCl₂). The folding buffer was sterilized with PVDF membrane filter unit 0.22 µm and kept at 4° C.

### E. Fresh PBMC

Blood samples were obtained from Tel Hashomer medical center blood bank and PBMC were isolated using Ficoll (Lymphoprep, Axis-Shield) density gradient centrifugation following the manufacturer’s protocol.

### F. Human CD8 T Cell Isolation

Isolation of human CD8 cells was performed via CD8+ T cells isolation Kit (Miltenyi Biotec, 130-096-495) following the manufacturer’s protocol.

### G. Aptamers List

Each aptamer was diluted to the desired concentration with the folding buffer. The aptamers were heated for 5 minutes at 95° C., followed by a rapid cooling for 10 minutes on ice, and room temperature (RT) incubation for 10 minutes. Folded aptamer was then added to the medium-suspended cells.

The following aptamers were used:

TABLE 5

Sequences related to CTL3 identification as T cell engager

| Aptamer name | SEQ ID NO: | Sequence 5′ to 3′ |
|---|---|---|
| Poly T 5′-Cy5-labelled | 82 | /5Cy5/TTTTTTTTTTTTTTTTTTTTTTTTTTTTTTTTTTTTTTTTTTTTTTTTTT |
| RND aptamer 5′-Cy5-labelled: | 83 | /5Cy5/CCGCGTCCGGACACCTAATTTGGTTCAAGAGCCGCCCGTAATTTCAGGT TCTCC |
| CTL3 5′-Cy5-labelled | 84 | /5Cy5/GCATACCTTTCGTATGCCTTTTTGACCCGTATTTTTGCCCTACCCTTCGG |
| Scrambled-CTL3 -A 5′-Cy5-labelled: | 85 | /5Cy5/GTTCTTATAATCGCCTCTGCGCTATGTTCTTGCTCGCCTTCCATATCGCT |
| Scrambled- CTL3-A | 86 | GTTCTTATAATCGCCTCTGCGCTATGTTCTTGCTCGCCTTCCATATCGCT |
| Scrambled-CTL3-B | 87 | TCTTTCGTTAGCGCTTCTCTCTTGCGATTCCGACCGCATATTCACGTCTT |

Lyophilized aptamers were kept in dark at RT until reconstituted in PBS-supplemented with 1 mM MgCl₂ to a concentration of 100 µM and stored at -20° C. in the dark.

### B. Experimental Methods

### A. Binding SELEX Protocol

The binding SELEX was conducted for 7 sequential rounds using CD8⁺ cells isolated from three healthy donors including two negative selections rounds (after rounds 3 and 4). The binding SELEX was performed as follows:

### Isolation and Preparation of CD8 T Cells for Individual SELEX Round

Prior to each round, CD8 cells were isolated, and recovered for 1 hour in a warm RPMI1640 (ATCC) at 37° C. Subsequently, cells were counted and seeded in a 1.5 mL Eppendorf tube at the following concentration:

TABLE 6

Amount of CD8 cells and negative selection cells in each binding SELEX round

| | | | | | | | | | |
|---|---|---|---|---|---|---|---|---|---|
| | Round 1 | Round 2 | Round 3 | Round 3 negative | Round 4 | Round 4 negative | Round 5 | Round 6 | Roun d 7 |
| Amount of CD8 cells | 10x10⁶ | 7x10⁶ | 4x10⁶ | | 3x10⁶ | | 2x10⁶ | 1x10⁶ | 1x10⁶ |
| Amount of CD8 negative cells | | | | 10x10⁶ | | 3x10⁶ | | | |

### Initial Library and Round-Enriched Library Preparation and Folding Protocol

The library is initially reconstituted to 1 mM. Working concentration in the first round was 14.3 µM, while in rounds 2-7, a concentration of 0.25-0.5 µM of enriched library was used. For each round the following components were used:

TABLE 7

Calculating library concentrations

| | | |
|---|---|---|
| Component | Concentration | Calculation |
| Enriched library | 0.25-0.5 µM | $\begin{matrix}{C}_{\text{μ} \text{} M} = \frac{{C}_{\frac{n g}{\text{μ} l}} \times 1 , 000}{330 g r / m o l e \times \left[90 n t \left(l i b l e n g t h\right)\right]} \\ {C}_{\text{μ} \text{} M} \times {V}_{E l u t i o n} = \left[0.2 u p t o 0.5\right] \text{μ} M \times {V}_{p o o l}\end{matrix}$ |
| Mix caps 5′+3′ | 50 uM | $\frac{{V}_{E l u t i o n} \times {C}_{E l u t i o n}}{50 μ \text{} M} \times 3 = {V}_{m i x c a p s}$ |
| Folding buffer | X10 | $\frac{{V}_{E l u t i o n} + {V}_{m i x c a p s}}{9} = {V}_{F B X 10}$ |

The libraries underwent DNA folding per the following protocol: were heated for 5 minutes at 95° C., followed by a rapid cooling for 10 minutes on ice, and room temperature (RT) incubation for 10 minutes. After folding, the following components were added in order to avoid non-specific nucleotide absorption and adjusted to a final volume as in Table 8:

TABLE 8

Calculating supplements

| | | | |
|---|---|---|---|
| Component | Concentration | Volume | Final concentration |
| tRNA | 10 mg/ml | 3.5 µl | 0.1 mg/ml |
| NaN₃ | 10% (in PBS) | 3.5 µl | 0. 1% |
| Medium+10% serum | N.A. | Adjust volume to 350 µl | N.A. |

### SELEX Round Duration and Washing Conditions

Once the enriched library round was folded, it was added to the isolated CD8 cells or to the negative cell population for a period of time as follows:

TABLE 9

Incubation time for each binding SELEX round

| | | | | | | | | | |
|---|---|---|---|---|---|---|---|---|---|
| | Round 1 | Round 2 | Round 3 | Round 3 negative | Round 4 | Round 4 negative | Round 5 | Round 6 | Roun d 7 |
| Positive SELEX | 1 h | 50 min | 40 min | | 30 min | | 20 min | 15 min | 15 min |
| Negative SELEX | | | | 1 h | | 1 h | | | |

After incubation, the cells were washed three times and centrifuged at 300 g for 5 min and the supernatant, “unbound to positive” fraction, was removed kept at -20° C. until NGS preparation. Cells were re-suspended with binding buffer and washed again. After the third wash, the cells were re-suspended in UPW, or binding buffer if a negative SELEX round was followed, and cells were lysed by heating for 95° C. for 10 min and centrifuged at full speed for 5 min at RT. The supernatant, “bound to positive” fraction, was removed, and used as a template for PCR reaction. If a negative SELEX round was followed, then the bound fraction was applied on CD8 negative cells for 1 hour at the same conditions described above and the collected fractions were called “unbound to negative” and “bound to negative”, respectively. After a negative SELEX round, the faction that was used for PCR amplification was the “unbound to negative” one.

### PCR Amplification Protocol

The “bound to positive” or the “unbound to negative” fraction was used as a template for asymmetrical PCR amplification. The PCR reaction was modulated for each round. The PCR components and the amplification protocol are shown in table 10 and table 11, respectively.

TABLE 10

PCR components

| Reagent | Stock | Volume |
|---|---|---|
| UPW | | Adjust to reaction final volume |
| Buffer | x5 | Adjust to reaction final volume |
| dNTPs mix | 10 mM | |
| Forward primer | 10 µM | |
| Reverse primer | 10 µM | |
| Template | | 10%-20% |
| DNA polymerase enzyme | | 1% |

TABLE 11

CR amplification protocol for enriched library

| Number of cycles | Temperature | Duration |
|---|---|---|
| 1 | 95° C. | 3 min |
| 30-36 | 95° C. | 30 sec |
| | Primer Tm-5° C. | 30 sec |
| | 68° C. | 30 sec |
| 1 | 68° C. | 4 min |

### PCR ssDNA Purification

The PCR products were purified using HPLC or by PCR ssDNA gel extraction kit (QIAEX II) followed by the manufacturer’s protocol. After purification, the DNA concentration was measured using NanoDrop, and the DNA was diluted for a new SELEX round.

### B. SELEX Libraries Binding Assay Protocol

Isolated CD8 cells or CD8-negative cell fraction (negative control) were counted, and 1x10⁶ cells were divided each into 1.5 mL eppendorf tube. Cells were centrifuge and washed once with binding buffer. The cells were re-suspended in 225 µL binding buffer supplemented with 0.01%Azide and 0.1% tRNA, and 25 µL folded Cy5-labelled aptamers were added to each treatment, followed by 1 hour incubations at 37° C. in the dark. Cells were washed 4 with binding buffer supplemented with 0.01%Azide and 0.1% tRNA, and fluorescence intensity was measured after each wash using flow cytometry (CytoFlex).

### C. Individual Aptamers Binding Assay

Isolated CD8 cells or Pan T cells, PBMCs or cell-line were counted, and 1x10⁶ cells were divided into each 1.5 mL eppendorf tube. Cells were centrifuge and washed once with binding buffer. The cells were re-suspended in 225 µL RPMI1640 supplemented with 10% human serum, and the folded Cy5-labelled aptamers were added to each treatment, followed by 1 hour incubations on ice in the dark. Cells were washed 4 times with cold medium and fluorescence intensity was measured using flow cytometry (CytoFlex).

### D. Thermofluorimetric Analysis (TFA)

TFA was used to determine the binding of CTL3 with its putative target Notch2.100 nM CTL3, 1 uM SYBR green I (sigma), Fc-Notch2 human (R&D Systems) or Fc-CD160 (abcam) at 20, 40, 80, 160, and 320 nM were mixed together and SYBR green and fluorescence was measured from Temp=25° C. to Temp=95° C. at 1 degree/min using RT-PCR, in triplicates. The subsequent experiment was done with 50 nM of either CTL3 (SEQ ID NO: 3), scrambled-CTL3-A (SEQ ID NO: 86), or scrambled-CTL3-B (SEQ ID NO: 87); 1 µM SYBR green I (sigma); Fc-Notch2 human (R&D Systems), Fc-Notch2 mouse (R&D Systems) or Fc-Notch2 rat at 25, 50, 100 and 200 nM similar to the former experiment.

### Example 3 - Identification of T Cell Engager Candidates via Binding SELEX

T cells have been established as core effectors for cancer immunotherapy, especially owing to their abundance, killing efficacy, and capacity to proliferate. T-cell engagers are bispecific molecules directed against a constant-component of the T-cell/CD3 complex on one end and a tumor-expressed ligand or antigen on the other end. This structure allows a bispecific T cell engager to physically link a T cell to a tumor cell, ultimately stimulating T cell activation and subsequent tumor killing (Huehls et al. (2015) Immunol. Cell Biol. 93:290-296; Ellerman D. (2019) Methods 154:102-117).

Selection of the Cytotoxic T Lymphocyte engaging aptamers was described herein. The cytotoxic T-lymphocyte arm was generated via Binding Cell-Selex using samples from multiple blood donors. The final lead was characterized for its binding to the target CD8⁺ T-Cells, its putative protein target identified via membrane protein array assay and was validated via thermofluorimetirc analysis.

This disclosure describes the identification and characterization of the cytotoxic T lymphocyte (CTL) engaging aptamers from a random library of 10¹⁵ potential aptamers using the Cell-SELEX methodology in a novel application.

In SELEX protocol, CTLs isolated from multiple healthy donors were used, sequentially in iterative selection rounds, to increase the likelihood of identifying aptamers that target widespread ligands, as oppose to individually-unique isoforms/mutants. To increase the specificity of the aptamer pool towards CTLs, negative selection was added in the form of CD8-negative PBMCs. In the final round of Cell-Selex, washing stringency of bound aptamer population was increased both in duration and in number of washes, in order to increase the affinity of potential aptamers in the final pool. After sequencing via next generation sequencing (NGS) and statistical analysis of enriched libraries throughout the selection process, putative binders were screened individually for their ability to bind primary CTLs. Top leads were tested for their capacity to promote target cancer cell cytotoxicity in the assembled structure of the bispecific aptamer, carrying a cancer-targeting aptameric arm. Concomitantly, Membrane Protein Array (MPA) platform (Tucker et al. (2018) Proc. Natl. Acad. Sci. U.S.A. 115:E4990-E4999) was used to deconvolute the putative targets of top leads, and the target of one leading aptamer “CTL3” was further validated, using thermofluorimteric analysis (Hu, Kim, & Easley (2015) Anal. Methods 7:7358-7362). The target of CTL3 was shown to be Notch-2, a membrane signaling receptor implicated in T-Cell-Mediated anti-tumor immunity and T-cell-based immunotherapy (Janghorban et al. (2018) Frontiers in Immunology 9:1649; Duval et al. (2015) Oncotarget 6:21787-21788; Ferrandino et al. (2018) Frontiers in Immunology 9:2165; Kelliher and Roderick (2018) Frontiers in immunology 9:1718; Weerkamp et al. (2006) Leukemia 20:1967-1977).

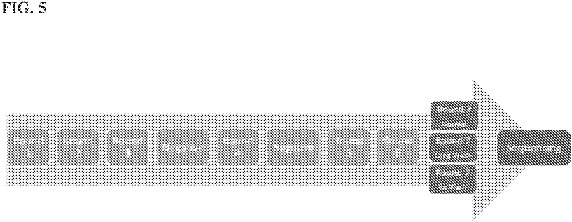

Binding Cell-SELEX was conducted using three healthy PBMCs donors for a total of seven rounds, as shown in FIG. 5. The use of multi PBMCs donors was carried out to ensure robustness of the aptamer-binding ability across different potential patients and not target a unique epitope expressed only in PBMCs of a single donor. Rounds 3 and 4 were followed by a negative selection round using CD8-negative PBMCs from donor 1 and 2.

### A. SELEX Rounds Comparative Assay

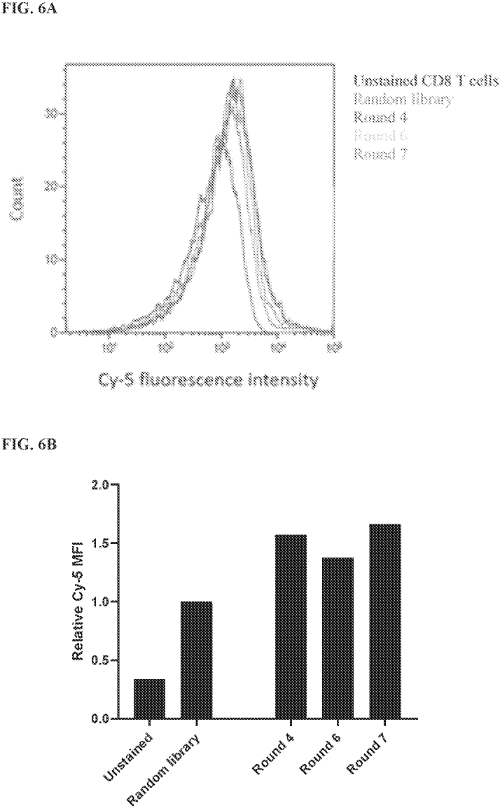

Libraries eluted from Rounds 4, 6 and 7 were tested for their binding affinity with isolated CD8 cells. Each round was amplified using 5′ primer labelled with Cy-5 followed by incubation with CD8 isolated cells for 1 hour. As shown in FIGS. 6A and 6B, the affinity of libraries from rounds 4, 6 and 7 was much higher than the random initial library used in the binding SELEX.

### B. NGS Results

The final round of Binding Cell-SELEX was repeated two more times with increased wash stringencies, once doubling the number of washing of unbound sequences (“6x Wash”, relative to the baseline 3x Wash), and a second time with increased incubation time after the final wash to allow aptamers with high K_{off} to be released into the medium and washed out (“long wash”) (see Table 12).

Enriched libraries for the 2^{nd}, 5^{th}, 6^{th} and three conditions of the 7^{th} round were sequenced (“bound”), as well as the supernatant of each round (“unbound”), via high-throughput sequencing using NGS Illumina NextSeq500.

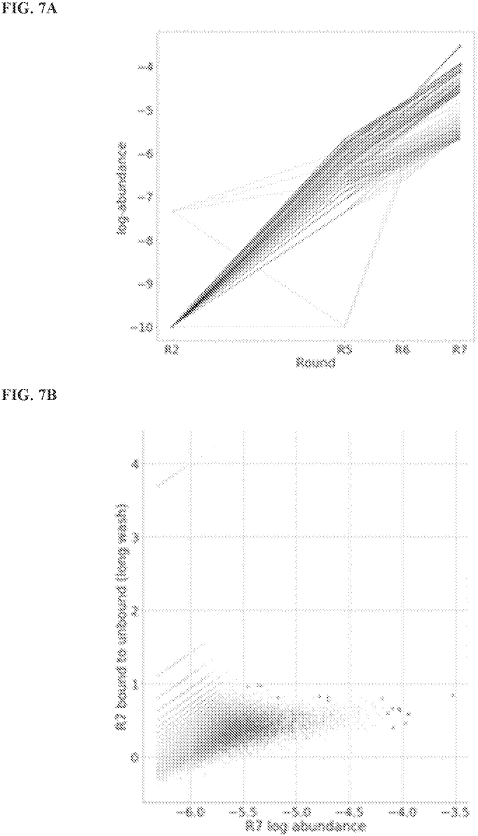

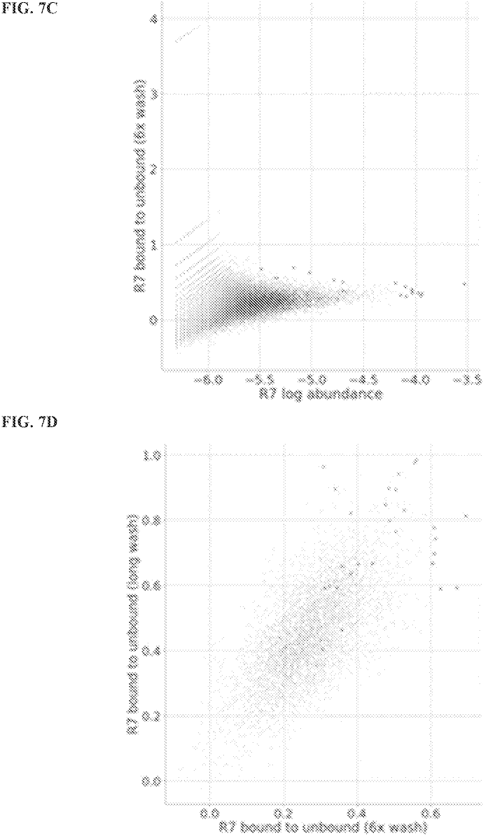

FIG. 7A shows the relative abundance of the most abundant sequences - the 10 most abundant in color and the rest in black (a total of 100 sequences). The results in FIG. 7A show increased abundance of top aptamers in the final enriched library, consistent with the increased binding results in FIGS. 6A and 6B.

Other than relative abundance, two additional measurements were calculated for each sequence in the final round 7 enriched library: the fraction of the sequence found in the cell bound population relative to the unbound population (supernatant) for the increased number of washes (6x Wash). The fraction of the sequence found in the cell bound population relative to the unbound population (supernatant) for the increased duration of the final wash (Long Wash).

The three measurements for each sequence in the final enriched library were plotted against each other (FIGS. 7B-7D) and 27 sequences were picked to be synthesized and tested individually for their binding affinity to CTLs (Table 13).

TABLE 12

Final Round permutations: wash stringency

| | | | |
|---|---|---|---|
| | Normal | 6 washes | Long wash |
| SELEX round duration | 15 min binding | 15 min binding | 15 min binding |
| Washes | 90 sec washX3 | 90 sec washX6 | 1. 90 sec washX2. 2. 30 min wash at 37° C. |
| Dissociation | 95° C. for 10 min | 95° C. for 10 min | 95° C. for 10 min |

TABLE 13

Tested CD8+ binding aptamers

| Aptamer name | SEQ ID NO: | Sequence 5′ to 3′ |
|---|---|---|
| CTL1 | 1 | TACGCGCAATTCGCCTTGTCGGTGATCTTCCTTTGAACTTGGGCAGTCTG |
| CTL2 | 2 | TGGCCTGGCCGTGTCGTCTGCTTTATAGTCGGTGATCCCTTGTGTTAATT |
| CTL3 | 3 | GCATACCTTTCGTATGCCTTTTTGACCCGTATTTTTGCCCTACCCTTCGG |
| CTL4 | 4 | TTTTTCGCTATCCAACCCTTCTTTCCAGCCTGCCAATCAGTCGGTGATCA |
| CTL5 | 5 | AGGGCAGTCCTGTATCTTAACATTCTCCTACATCCGTAAGTCGGTGATCC |
| CTL6 | 6 | GGGCTAAGAGTCTCTATTGTCGGCAGTCGTCTAATATTTCCCGTCCAATT |
| CTL7 | 7 | ACTTCCGGTGATTTGATTTCACTTCCTGGGCAGTCAATGTGATTCTCTAC |
| CTL8 | 8 | GGTCAGTCGCCTTTGTCGGTGATGTACTCGCGCAGTCGGGTTCCCCTTAA |
| CTL9 | 9 | GGGTCTGTTGGTCCTAGGGCAGTCGTACTTCTAATTCTTGTCCCGATGAT |
| CTL10 | 10 | CTTGTCGGTGATCTATAGTCGGTGATATATTTTGTCCTATGGTAGTCGAT |
| CTL11 | 11 | GGGCTCATGGGCAGTCTTTTTACTACCTCCTATTTACGTATCCCGCTCCT |
| CTL12 | 12 | CACCCGCGCATTTCCCCCCAGTCGGTGATTCTTATATGTACCTGTTCCTC |
| CTL13 | 13 | GGGCACGTCCATTCGCGTTTTTGTTCCGTTTCTCCCTTTTTGGATTTTGC |
| CTL14 | 14 | CAGTCGGTGTCACTCCAGCGGTCGGTTCACTCCACATTCTCCCATCTGTC |
| CTL15 | 15 | GGCAGTCACCATTCTCTTTGGGCAGATTGTCTCTCATCCATATGTCTCCT |
| CTL16 | 16 | CTACCTCCTTAGTCGGTGATTCGATCTATGGGCCTAACTGCCTTCTCTGT |
| CTL17 | 17 | GGGATGCGGGGCCCCGTTCTTTTTGTCTCTCATTTTGTCACTTTTTTTGT |
| CTL18 | 18 | GGTCAGTCCCTTCGGCATGTCGGGATTCCCTCTTTTCGCCTCGTTTCTTT |
| CTL19 | 19 | GGCTGTCGAACTTTCTCCCTCCCACCGCAGTCGGCCCCTCATCAGTCGTA |
| CTL20 | 20 | ACTTCCGGTGATTTGATTTCACTTCCTGGGCAGTCAATGTGATTCTCTAC |
| CTL21 | 21 | ACGTCTGTCGGTGACCTGTAATAGTTTATGTCGGTGATACAGCTTTCCCT |
| CTL22 | 22 | CTGTCGGTGATCATATAACGCAGTCGGTGTAGTTTAATCCCACTCCCCTA |
| CTL23 | 23 | GGCCAGTGTCCCAGTCGTGATTGTAATATTAGATTCTTTGTGGCAGTCGT |
| CTL24 | 24 | ACTCGTCGGTGATTTTAGACCTTTCTCGGTGATCAACACGTCATGCTATT |
| CTL25 | 25 | GCCTCGATATCCTCAGGAGTCGGTGTTTCATTCAATCGTCGGTGATAAAT |
| CTL26 | 26 | GGTCAGTCCGTATACCGCCAATCCGAACCGCAGTCGGTGTCCGCTTTTAC |
| CTL27 | 27 | TCGGGTTAGATGTCGGTCCCACTATATGTCGGTGATCTAATATTGAACTT |

### C. Individual Aptamers Validation

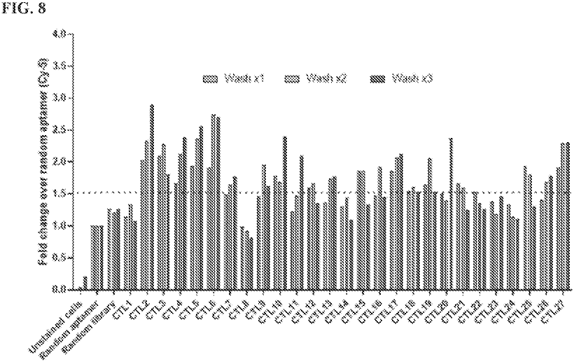

Aptamers selected from the statistical analysis were synthesized with a 5′ Cy5 fluorescence label and screened for their binding to isolated CD8 cells. A positive binding threshold was determined as above 1.5 folds over random aptamer sequence (FIG. 8).

### Example 4 - T Cell Engager Characterization (of Example 3)

### A. CTL3 Sequence and Structure

CTL3 sequence:

5’-GCATACCTTTCGTATGCCTTTTTGACCCGTATTTTTGCCCTACCCTTCGG-3’
(SEQ ID NO: 3)

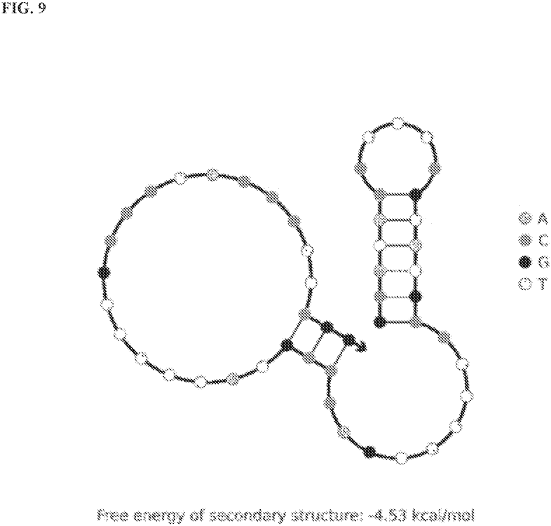

The predicted structure of CTL3 by Nupack software is shown in FIG. 9.

### B. CTL3 Binding Assay via Flow Cytometry

### 1. CTL3- Binding to Human PBMCs

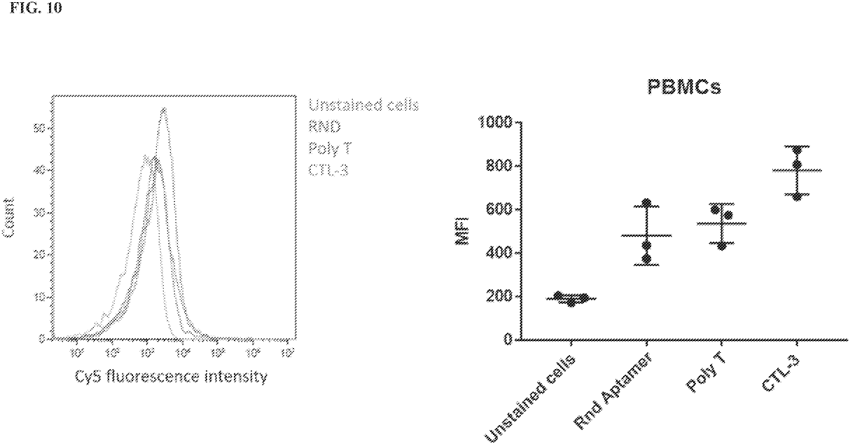

To visually demonstrate the binding of the selected aptamer to its target cell type and to better understand its specificity, human frozen PBMCs from several different donors were thawed and stained with CTL3 Cy5-labelled aptamer as well as Cy5-labelled negative controls, Poly-T and random (RND) aptamer sequence. CTL3 aptamer exhibited higher binding to total PBMCs compared with random aptamer control and Poly T aptamers (FIG. 10).

To better understand the specificity of CTL3 aptamer, CD8-staining was used together with SSC/FSC to differentiate between PBMC subpopulations.

Human PBMCs from three different healthy donors were tested for binding with Cy5-labelled aptamers (250 nM) followed by CD8 antibody staining.

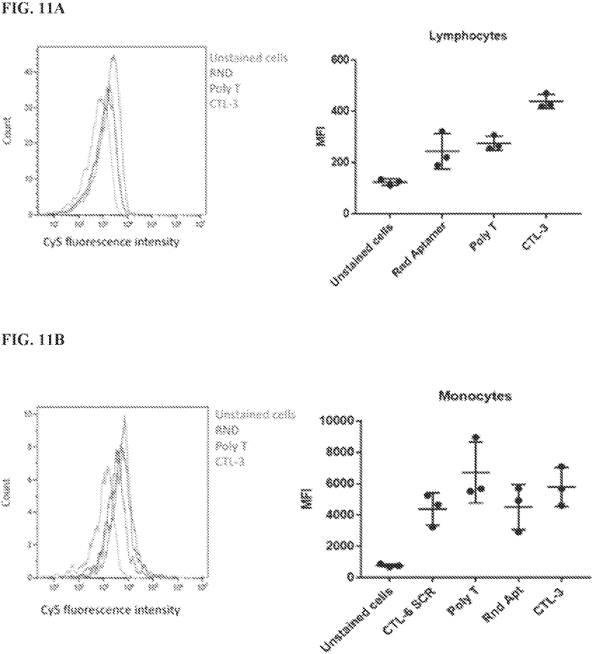

Binding of CTL3 to lymphocyte population was greater compared to RND control and Poly T aptamers, while no significant binding differences between CTL3, RND control and poly T aptamers to monocytes cells were observed (FIGS. 11A and 11B). Within the lymphocytic population however, CTL3 was found to bind both CD8-positive and CD8-negative lymphocytes (FIGS. 11C and 11D).

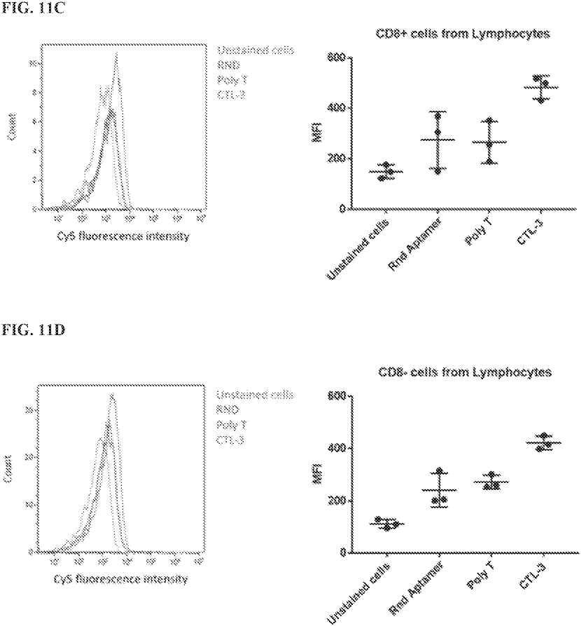

A scramble sequence (SCR) containing the same nucleotides ratio as CTL3 was designed. CTL3 demonstrated binding even in comparison with this stringent control (FIGS. 12A and 12B).

### 2. Binding Assay With Isolated CD8 Cells

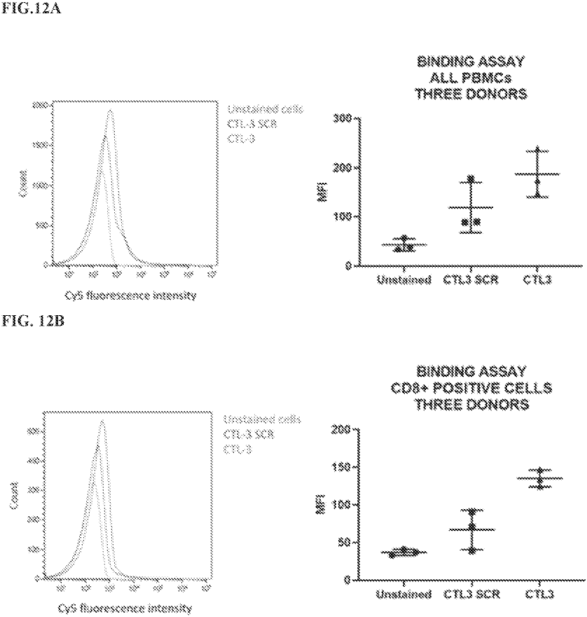

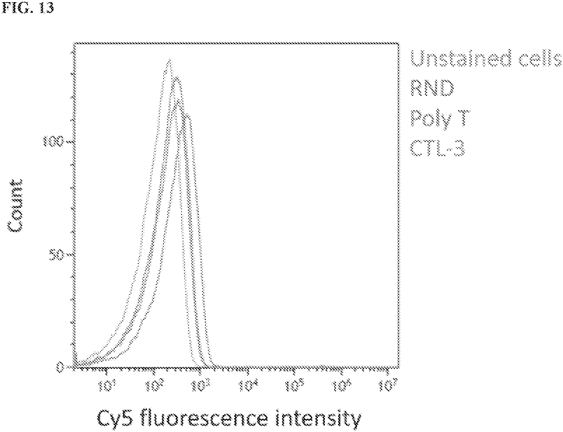

In order to rule out reduced signal due to a mixed PBMC population, CD8 T cells were isolated prior to the assay and CTL3 binding was measured directly on this subpopulation. FIG. 13 displayed representative results from a single experiment. The results nevertheless, were consistent with the PBMCs binding results.

### 3. CTL3 Binding to Expanded and Stimulated T Cells

CTL3 aptamer was subjected to target de-orphaning described herein, and Notch2 was identified and validated as the aptamer’s target. Notch2 surface expression is dynamically regulated during T cell development and activation (Duval et al. (2015) Oncotarget 6:21787-21788; Ferrandino et al. (2018) Frontiers in Immunology 9:2165; Kelliher and Roderick (2018) Frontiers in immunology 9:1718; Weerkamp et al. (2006) Leukemia 20:1967-1977).

To measure the dependency of CTL3 binding on the active state of the target cells, an exploratory experiment was performed in which T cells were isolated from one donor’s PBMCs, via pan-T isolation kit, and activated via a combination of anti-CD3 (1 µg/µL) & anti-CD28 (1 µg/µL) antibodies for 48 hr followed by IL-2 (300 Unit) for 9 days. Binding was measured 11 days after the initial activation. Under these conditions, no significant increase in CTL3 binding ability was observed compared to binding with all hPBMCs or isolated CD8 T cells (FIGS. 14A and 14B)

### C. Target Deconvolution of CTL3 by Membrane Proteome Array

The Membrane Proteome Array (MPA) is a platform developed by Integral Molecular Inc (Philadelphia, PA, US) for profiling the specificity of antibodies and other ligands that target human membrane proteins. The MPA can be used to determine target specificity and deconvolute orphan ligand targets (Tucker et al. (2018) Proc. Natl. Acad. Sci. U.S.A. 115:E4990-E4999).

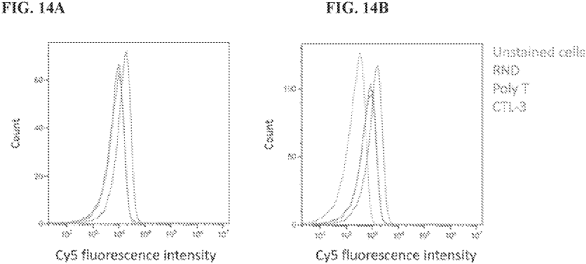

The platform uses flow cytometry to directly detect ligand binding to membrane proteins expressed in unfixed cells (see FIG. 15). Consequently, all target proteins have native conformations and appropriate post-translational modifications.

CTL3 aptamer was tested for reactivity against a library of over 5,300 human membrane proteins, including 94% of all single-pass, multi-pass and GPI-anchored proteins. Identified targets were validated in secondary screens to confirm reactivity.

A high-throughput cell-based platform is used to identify the membrane protein targets of ligands. Membrane proteins are expressed in human cells within 384-well microplates, and ligand binding is detected by flow cytometry, allowing sensitive detection of both specific and off-target binding.

Each well on the matrix plate contains 48 different overexpressed protein constituents. Each protein is represented in a unique combination of two different wells of the matrix plate, as it is contained within a “row” pool and a “column” pool. Test CS aptamer was added to MPA matrix plates at predetermined concentrations, washed in 1× PBS, and detected by flow cytometry.

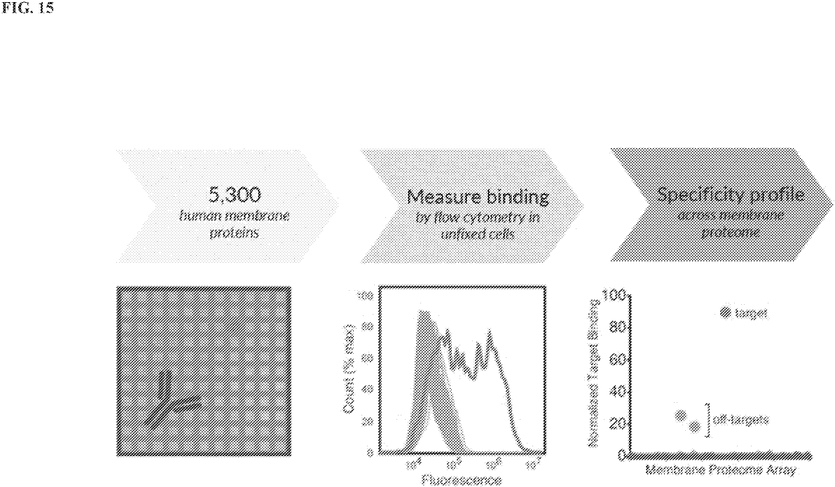

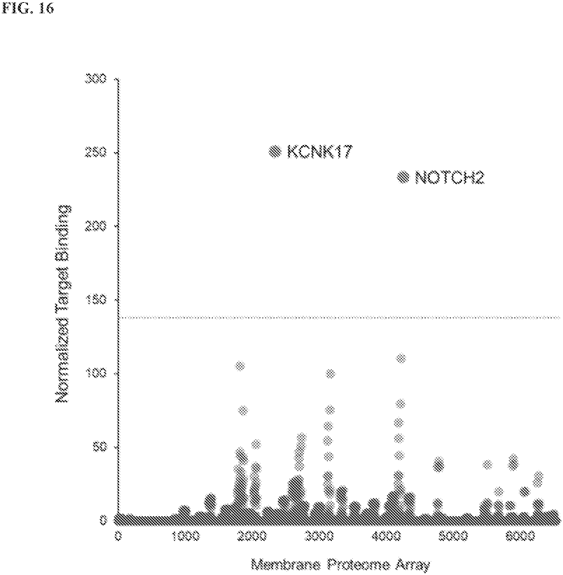

CTL3 aptamer target hits were then identified by detecting binding to overlapping pooled matrix wells emanating from the same transfection plate, thereby allowing specific deconvolution. The screening yielded two potential hits: KCNK17 and Notch2 (FIG. 16).

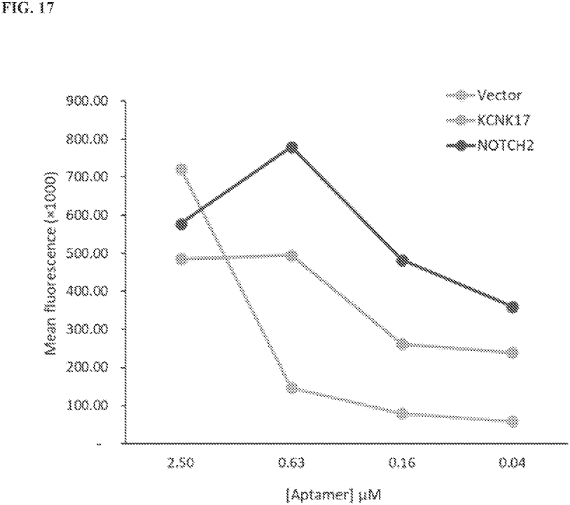

To validate protein targets identified using the MPA, HEK 293T cells were transfected with plasmids encoding the respective targets, or vector alone (pUC; negative control) in 384-well format. After incubation for 36 hours, four 4-fold dilutions of CTL3 were added to transfected cells followed by detection of aptamer binding using a high-throughput immunofluorescence flow cytometry assay. Average mean fluorescence intensity (MFI) values were determined for each aptamer dilution (FIG. 17). Notch2 and KCNK17 (a potassium channel subfamily K member 17) have been validated to generate a concentration-dependent binding curve substantially higher than the negative control vector’s.

### D. Binding of CTL3 to Recombinant Notch2 by Thermofluorimetric Analysis

While no T cell related literature was found for KCNK17, the Notch pathway regulates CD8 T cells in multiple ways. CD8-specific deletion of Notch2, but not Notch1 for example, led to increased tumor size and decreased survival after tumor-inoculation into mice, implying a potential contribution of this receptor to an antitumor immune response (Sugimoto et al (2010) J immunol ; Mathieu et al (2012) Immunol. Cell Biol. 82-88; Tsukomo and Yasutomo (2018) Front. Immunol. 9, 1-7).

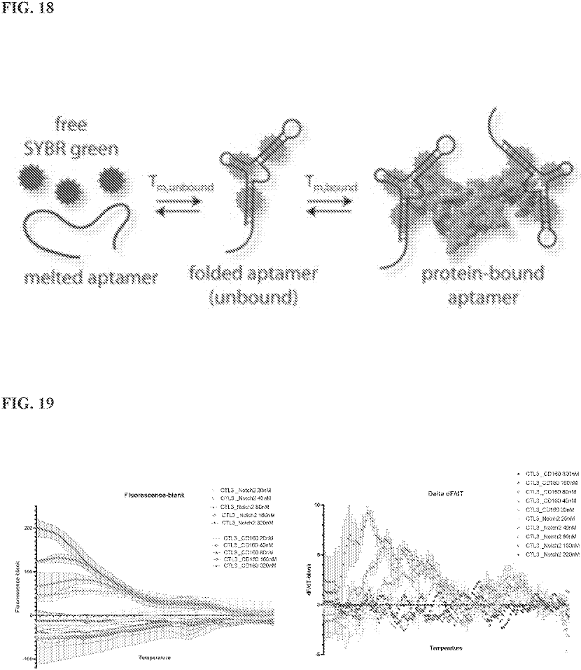

In order to provide direct biochemical evidence that Notch2 is the binding target of CTL3, Thermofluorimetric Analysis (TFA) assay was used. In TFA, DNA-intercalating dyes were used to determine binding constants between DNA-aptamers and target proteins by measuring the temperature-dependent fluorescence of aptamers labeled with SYBR, an intercalating dye, with and without their prospective protein binding partners (Hu, Kim and Easley (2016) HHS Public Access. 7:7358-7362). Upon gradual heating of the aptamer-dye solution, the duplex parts in the aptamer were denatured and the dye was released back to the solution, which highly reduced its fluorescence. Since the aptamer 3D conformation was greatly stabilized upon binding to its respective target protein, the temperature-dependent fluorescence of aptamer-dye complexes varied greatly with and without the putative protein binding partner (FIG. 18).

A Tₘ melting curve profile was generated by measuring SYBR green fluorescence during temperature gradient, to monitor aptamer-protein complexes in the presence of different concentrations of either Notch2 or the non-specific control (CD 160 protein). Only upon the addition of increasing concentrations of Notch2, and not CD160, a dose-dependent change in CTL3-associated fluorescence was measured (FIG. 19). When looking at the total fluorescence graph, high fluorescence intensity can be seen at 25° C., however, when examining the derivative rate of change of frequency (dF/dT) curves, the temperature-dependent intensity reached a maximum at 37° C.

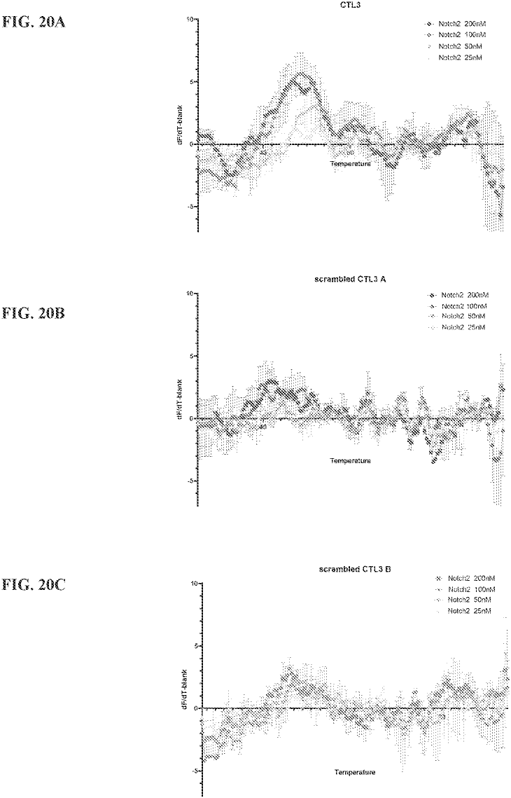

CTL3-Notch2 binding was compared with two scrambled sequences (named scrambled CTL3- A and scrambled CTL3-B) which contain the same base composition. It can be seen from FIG. 20A that CTL3 exhibits a dose-response curve by increasing the concentration of Notch2. This phenomenon is not seen with the scrambled strands, suggesting specific reaction between CTL3 and Notch2 that reaches saturation between 100-200 nM of protein.

In conclusion, in the presence of the DNA intercalating dye, Notch2 protein-bound CTL3 aptamer exhibits a change in fluorescence intensity compared to the intercalated, unbound aptamer. This intensity change does not occur when CD160 is added instead of Notch2, or when scrambled sequences are added.

In contrast to human recombinant Notch2 for which CTL3 aptamer has demonstrated a clear concentration-dependent binding (FIG. 21A), no such pattern was clearly demonstrated for mouse or for rat Notch2, implying less specific binding by CTL3 (FIG. 21B and FIG. 21C).

### Example 5 —Materials and Methods for Examples 6-7

### A. Materials

### A. Random Library

Random library 9.0 (“Lib 9.0”) was purchased from IDT. The library contains a vast repertoire of approximately 10¹⁵ different 40nt-long random sequences flanked by two 20 nt unique sequences at the 3′ and 5′ acting as a primer for PCR amplification during the SELEX procedure. The lyophilized library was reconstituted in ultra-pure water (UPW) to a final concentration of 1 mM. The random library sequence was: 5′-TCACTATCGGTCCAGACGTA-40N-TATTGCGCCGAGGTTCTTAC-3′ (SEQ ID NO.117), where N represents a random oligonucleotide selected from a mixture of equally represented T, A, C, and G nucleotides (1:1:1:1 ratio).

### Pre-SELEX Preparation

Following reconstitution, the library underwent QC validation for size exclusion using HPLC ProSEC 300S column (Agilent).

### B. Library Primers and Caps

A set of 20 nt primers and caps were purchased from IDT (Table 14). Caps were used to hybridize to the Library’s primer sites during incubation with cells in order to refrain from the possibility of primer sequences interacting with the random 40 nt sequence site. A mixture of 3′ and 5′ caps (Table 14) in each SELEX round was used in a 3:1 caps-to-library ratio.

The forward primer was purchased from IDT labelled with Cy-5 at the 5′ site for sequence amplification that was detected in a fluorescence assay. The lyophilized primers were reconstituted in ultra-pure water (UPW) to a concentration of 100 µM.

TABLE 14

Random library, primers and caps sequences

| Auxiliary sequences | SEQ ID NO: | Sequence 5′ to 3′ |
|---|---|---|
| Random Library | 117 | TCACTATCGGTCCAGACGTA-40N-TATTGCGCCGAGGTTCTTAC |
| Forward Primer | 118 | TCACTATCGGTCCAGACGTA |
| Forward labeled Cy-5 | 119 | /Cy5/TCACTATCGGTCCAGACGTA |
| Reverse/3′ cap | 120 | GTAAGAACCTCGGCGCAATA |
| 5′ cap | 121 | TACGTCTGGACCGATAGTGA |

### C. Aptamer Folding Buffer

Phosphate-buffered saline (minus Magnesium and Calcium) was supplemented with 1 mM Magnesium Chloride (MgCl₂). The folding buffer was sterilized with PVDF membrane filter unit 0.22 µm and kept at 4° C.

### D. PBMC

PBMC were isolated using Ficoll (Lymphoprep, Axis-Shield) density gradient centrifugation following the manufacturer’s protocol.

Frozen Cynomolgus Monkey PBMCs (NHP-PC001) were purchased from Creative Biolabs.

### E. Human PanT and B Cell Isolation

Isolation of human Pan T cells was performed by using Pan T cells isolation kit (Miltenyi Biotec, 130-096-535) following the manufacturer’s protocol. Isolation of human Pan B cells was performed by using Pan B cells isolation kit (Miltenyi Biotec, 130-101-638) following the manufacturer’s protocol

### F. Antibodies, Proteins and Enzymes

αCD3ε-FITC (Cat. #130-113-690) /APC (Cat. #130-113-687) /VioBlue (Cat. #130-114-519) /APC-Vio770 (Cat. #130-113-688), αCD4-FITC(Cat. #130-114-531) , αCD8-FITC(Cat. #130-113-719) / PE-Vio770 (Cat. #130-113-159) and matching isotype controls were purchased from Miltenyi Biotech. αCD3ε, OKT3 clone (Cat. #317302) was purchased from BioLegend.

Recombinant Human CD3 epsilon protein (Fc Chimera His Tag) (ab220590), Recombinant Cynomolgus CD3 epsilon protein (Fc Chimera His Tag) (ab220531), and Recombinant Mouse CD3 epsilon protein (His tag) (ab240841) where purchased from Abcam. Human IgG1 isotype was used as a negative counter selection (InVivoMAb, BE0297).

Protein G magnetic beads purchased from ThermoFisher ( 88847).

Herculase II Fusion DNA Polymerase (600675) that is used for Asymetric PCR (A-PCR) purchased from Agilent and real-time-PCR iTaq Universal SYBRGreen Supermix (1725124) purchased from BIO-RAD.

### G. Cell-Lines

Jurkat, Daudi and Kasumi-1 cell-lines were purchased from ATCC. Jurkat cell (ATCC TIB-152), Daudi cells (ATCC CCL-213) and Kasumi-1 (ATCC CRL-2724) were grown in RPMI-1640 supplemented with 10% fetal calf serum (FCS) and 1% Penicillin and streptomycin (Pen/Strep). All cells were cultured at 37° C. and 5% CO2.

### H. Aptamers

Each aptamer was diluted to the desired concentration with the folding buffer. The aptamers were heated for 5 minutes at 95° C., followed by a rapid cooling for 10 minutes on ice, and room temperature (RT) incubation for 10 minutes. Folded aptamer was then added to the medium-suspended cells.

Lyophilized aptamers were kept in dark at RT until reconstituted in PBS-supplemented with 1 mM MgCl₂ to a concentration of 100 µM and stored at -20° C. in the dark.

### B. Experimental Methods

### A. Binding SELEX Protocol

The binding SELEX was conducted for 11 sequential rounds using CD3ε-Fc protein coupled to protein G magnetic beads (Positive selection ), IgG1 protein coupled to protein G magnetic beads or with beads only (Negative selections, starting from round 3 onwards).

### I. Beads-Protein Complex Preparation

Magnetic protein G beads were vortexed and washed once with PBS and then mixed with 100ul of protein for 10 min at RT under gentle shaking condition. Then, the beads were separated by a magnet, the supernatant was discarded and the beads re-suspended with 350 ul of Folding buffer x1 containing 2% BSA.

For verification of the beads-protein complex formation, a small sample (before DNA added) was treated with FC-blocker (Miltenyi), stained with αCD3ε and analysed by flow cytometry

### II. Initial Library and Enriched Round Library Preparation and Folding Protocol

The library is initially reconstituted to 1 mM. Working concentration in the first round was 14.3 µM, while in rounds 2-11, a concentration of 0.25-0.5 µM of enriched library was used. For each round the following components were used:

TABLE 15

Calculating library concentrations

| | | |
|---|---|---|
| Component | Concentration | Calculation |
| Enriched library | 0.25-0.5 µM | $\begin{matrix}{C}_{\text{μ} \text{} M} = \frac{{C}_{\frac{n g}{\text{μ} l}} \times 1 , 000}{330 g r / m o l e \times \left[90 n t \left(l i b l e n g t h\right)\right]} \\ {C}_{\text{μ} \text{} M} \times {V}_{E l u t i o n} = \left[0.2 u p t o 0.5\right] \text{μ} \text{} M \times {V}_{p o o l}\end{matrix}$ |
| Mix caps 5′+3′ | 50 uM | $\frac{{V}_{E l u t i o n} \times {C}_{E l u t i o n}}{50 μ \text{} M} \times 3 = {V}_{m i x c a p s}$ |
| Folding buffer | X10 | $\frac{{V}_{E l u t i o n} + {V}_{m i x c a p s}}{9} = {V}_{F B X 10}$ |
| Folding buffer | X1 | Adjust volume to 350ul |

The libraries underwent DNA folding per the following protocol: were heated for 5 minutes at 95° C., followed by a rapid cooling for 10 minutes on ice, and maintained until use at 4° C.

### III. SELEX

Once the enriched library was folded, 350ul of enriched library rounds was added to 350ul of CD3ε-FC-bead (positive selection rounds 1-11) or to Beads only /IgG₁-beads complex (counter selection, rounds 3-11). Incubation time, protein amount and wash steps varied by the SELEX rounds.

In positive selection, the supernatant, “unbound to positive” fraction, was removed kept at -20° C. until NGS preparation. For washes, the beads were precipitated with a magnet, the supernatant was discarded and the beads were re-suspended with 1ml of folding buffer x1. After the washing step, the beads suspend in 300 ul ultra-pure water (UPW) and the DNA eluted at 95° C. for 10 min. Finally, the beads precipitated with magnet, and supernatant “bound to positive” was collected for the PCR stage.

If a negative SELEX round was implemented, than the 350 ul of enriched library rounds was added to 350 ul of beads only / IgG beads complex and the supernatant collected fractions proceeded to positive selection stage. The binding fraction to the negative samples, called “bound to negative”, were eluted and kept at -20° C. until NGS preparation.

### Iv. PCR Amplification Protocol

The eluted DNA fractions (“bound” and “unbound” )were used, each, as a template for Asymmetrical PCR (A-PCR) amplification. The PCR reaction was modulated for each round. The PCR components and the amplification protocol are shown in table 16 and table 17, respectively.

TABLE 16

PCR components

| Reagent | Stock | Volume |
|---|---|---|
| UPW | | Adjust to reaction final volume |
| Buffer | x5 | x1 |
| dNTPs mix | 10 mM | 0.8 mM |
| Forward primer | 10 µM | 2.5 uM |
| Reverse primer | 10 µM | 0.25 uM |
| Template | | 15% |
| DNA polymerase enzyme | | 1% |

TABLE 17

PCR amplification protocol for enriched library

| | | |
|---|---|---|
| Number of cycles | Temperature | Duration |
| 1 | 95° C. | 3 min |
| 18-36 | 95° C. | 30 sec |
| | 58° C. | 30 sec |
| | 72° C. | 30 sec |
| Final | 4° C. | ∞ |

### V. PCR ssDNA Purification

The PCR products were concentrated with 10 K Amicon (Millipore, UFC5010BK) and purified using HLPC ProSEC 300S size exclusion column (Agilent). After purification, the DNA underwent buffer exchange with ssDNA clean kit (ZYMO, D7011), concentration was measured using NanoDrop and the DNA was diluted for a new SELEX round.

### B. Assessment of Librarypools Binding to Target Protein by Real-Time-PCR

Magnetic protein G beads were vortexed and washed once with PBS and then re-suspended with protein (CD3ε or IgG₁) for 10 min at RT under gentle shaking condition. Then, the beads were precipitated under the magnetic field, the supernatant was discarded and the beads re-suspended with 125 ul of Folding buffer x1 and 2% BSA. Next, the library pools from rounds 3, 6, 9, 11, and the initial random library were folded (95℃ 5 min, ice 10 min, and maintenance at 4℃). 125 ul of each of the folded DNA libraries was mixed with the beads-protein complex for 1 hr at 4℃ in a gentle shaking. After incubation, the beads were precipitated with a magnet and washed 3 times with 1 ml folding buffer. Finally, the DNA binding fraction was eluted at 95℃ with 100 ul UPW for 10 min and subsequently used as a template in real-time-PCR with SYBRGreen Supermix (BIO-RAD).

### C. Assessment of Individual Aptamers Binding to Target Protein Protein-Aptamers Binding Assay by HPLC

1 µM of folded Cy5 labeled aptamer was mixed with 5 µM of protein to a final volume of 60 ul and incubated for 1 hr at 4℃ or 37℃. Next, to detect the Cy-labelled aptamers, samples were analyzed at 570 nm absorption via HPLC ProSEC 300S size exclusion column (Agilent).

### D. Assessment of Individual Aptamers Binding to Cells by With Flow Cytometry

0.5-2 x10⁶ cells (isolated Pan T cells, B cells, hPBMCs, Cynomolgus PBMCs, Jurkat, and Daudi) were washed and re-suspended in 0.2-1.ml folding buffer that contains 0.1% BSA and 0.01% tRNA.

0.25-1.25 uM of single DNA candidate were fluorescently labelled by mixing with CpG′-Cy5 tag (1:1 ratio) and folded (95℃ 5 min, ice 10 min, and maintenance at 4℃). Next, the labelled DNA aptamers were incubated with the cells for 1 hr at 4℃ or 37℃ in V shape 96 well plate under gentle shaking conditions (hPBMCs and Cyno PBMCS were added αCD8/ αCD4 in the final 15 min of incubation). After incubation, cells were washed 3 times with folding buffer X1 and analysed after each wash using flow cytometry (CytoFlex).

### E. Competitive CD3 Epsilon Epitopes Binding Assay

0.25x10⁶ Jurkat cells were washed once, re-suspended in folding buffer x1 containing 0.1% BSA and 0.01% tRNA and incubated for 15 min with 1:20 dilution of αCD3 clone OKT3 (BioLegend, 317302) or αCD3 clone REA613 (Miltenyi, 130-114-519) or with buffer. Next, 0.25 µM of folded Cy5 labelled aptamers were incubated with the cells for 1 hr at 37℃ under gentle shaking condition. After incubation, cells were washed 3 times with folding buffer X 1 and analysed after each wash using flow cytometry (CytoFlex).

### F. CS6 Effective Concentration 50 (EC50) Quantification

5 x10⁴ Jurkat cells were washed and re-suspended in x1 folding buffer that contain 0.1% BSA and 0.01% tRNA. 0.1-80 nM of CS6 aptamer were labelled with CpG′-Cy5 tag (1:1 ratio) and folded (95℃ 5 min, ice 10 min, and maintenance at 4℃). Next, the DNA aptamers were mixed with the cells and incubated for 1 hr at 37℃ in V shape 96 well plate under gentle shaking conditions. After incubation, the cells were washed twice with folding buffer X1 and analysed via flow cytometry (CytoFlex).

### Example 6 - Identification of CD3 -Targeting Aptamer Via Binding SELEX

A significant optimization step of the drug candidate was carried out via the replacement of the above-mentioned T cell engager with a novel aptamer targeting CD3 epsilon ligand on the surface of T cells.

Selection of the CD3 binding aptamers was described herein. The T cell targeting aptamers were identified via Binding SELEX and Hybrid Binding Cell-SELEX using recombinant CD3e protein and recombinant protein plus T cells, respectively. The final lead was characterized for its binding to the target protein and T-Cells.

This disclosure describes the identification and characterization of the T cell engaging aptamers from a random library of 10¹⁵ potential aptamers using the SELEX methodology in a novel application. This aptamer moiety, as part of the bispecific therapeutic entity was designed to be constant across different patients.

Binding SELEX was conducted using recombinant Human CD3 epsilon protein Fc chimera for a total of eleven (11) rounds. For counter negative selection, either beads only (rounds 1-6) or beads conjugated to Human IgG₁ (rounds 7-11) were used in order to rid of all aptamers which bind non-specifically to the magnetic beads or to the Fc component of the recombinant protein (FIG. 22). After round 11 of the SELEX, enriched aptamer libraries were subjected to sequencing and analysis via specific algorithm. Single candidates were identified and undergo verification.

FIG. 22B depicts the SELEX stages: counter selection starts with protein G magnetic beads (1) that were conjugated to IgG1 (2) and incubated with DNA aptamer library pool from the previous stage (3). Next, unbound DNA aptamers were collected for positive selection (4) and were incubated with FC-CD3ε-conjugated beads (5) here, the bound fraction (6) underwent PCR amplification and HPLC purification for the next round.

### 1. SELEX Rounds Comparative Assay

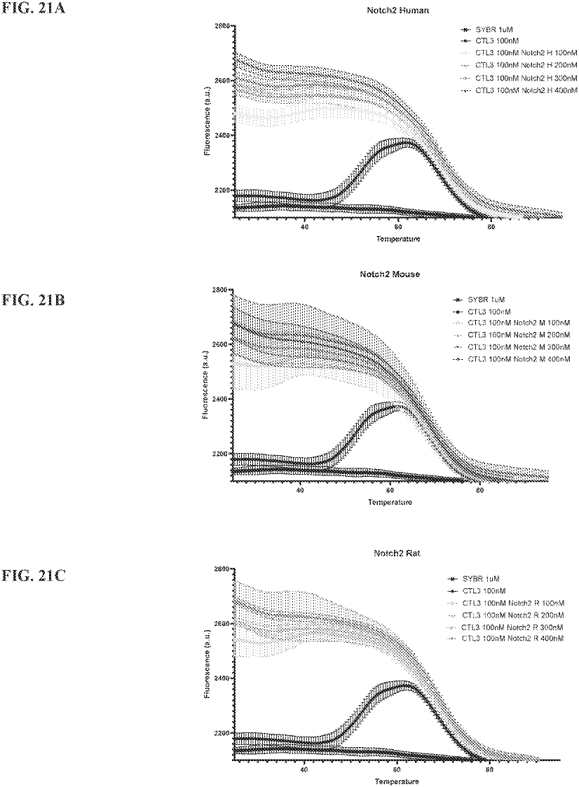

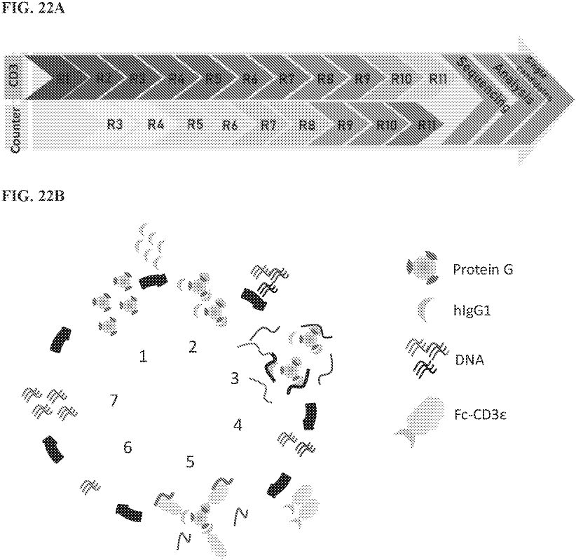

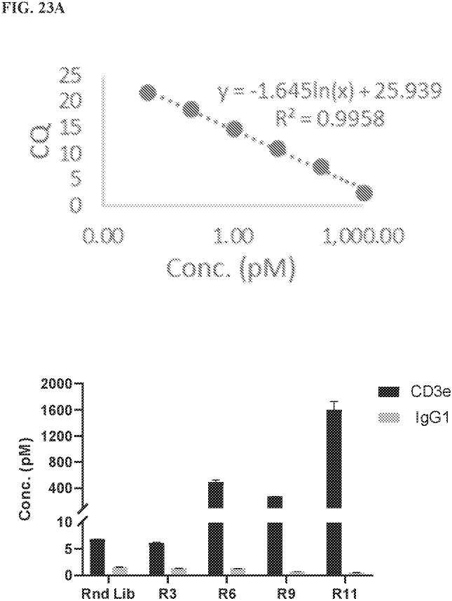

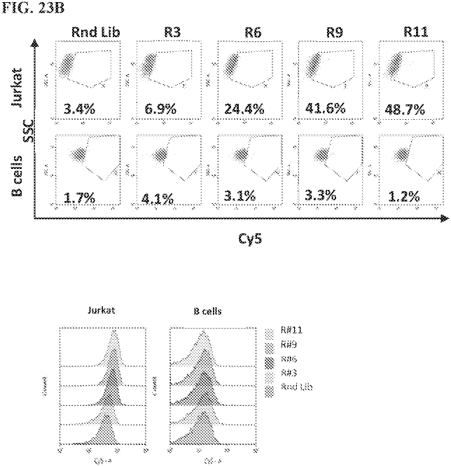

Original random library ‘No.9.0’ and library pools eluted from rounds 3, 6, 9 and 11 were tested for their binding to hCD3ε. Each round was amplified by PCR using 5′ primer labelled with Cy-5 following incubation with Beads-Fc-CD3ε complex for 1 hr at 4℃. As a negative control, the variant pools where incubate with Beads-IgG1 complex (FIG. 23A). The amount of amplified DNA, which was precipitated with the target protein, was found much higher in libraries from rounds 6, 9 and 11 than in the random initial library used in the binding SELEX. The results showed specific and strong enrichment as of round six compared with the initial library. Further, there was another increment in the specific binding observed in round 11.

After demonstrating round-to-round enrichment using the recombinant CD3 protein, we tested whether such enrichment is observed also in a whole-cell context. Jurkat T cells were incubated with the same Cy5 tagged library pools, washed, and analysed by flow cytometry. As a negative control, isolated Pan B cells were used (FIG. 23B).

Similarly to the protein data, a specific and strong round-to-round enrichment for the target cells was demonstrated.

### 2. NGS Results

Enriched libraries eluted in rounds 8,9,10 and 11 (“bound”), as well as the supernatant of positive selection rounds (“unbound”), were subjected to sequencing using the high-throughput NGS Illumina NextSeq500.

Post sequencing, the data was analyzed via an algorithm which allocated single candidates for downstream binding assays. The algorithm utilizes statistical estimators, tests, and metrics.

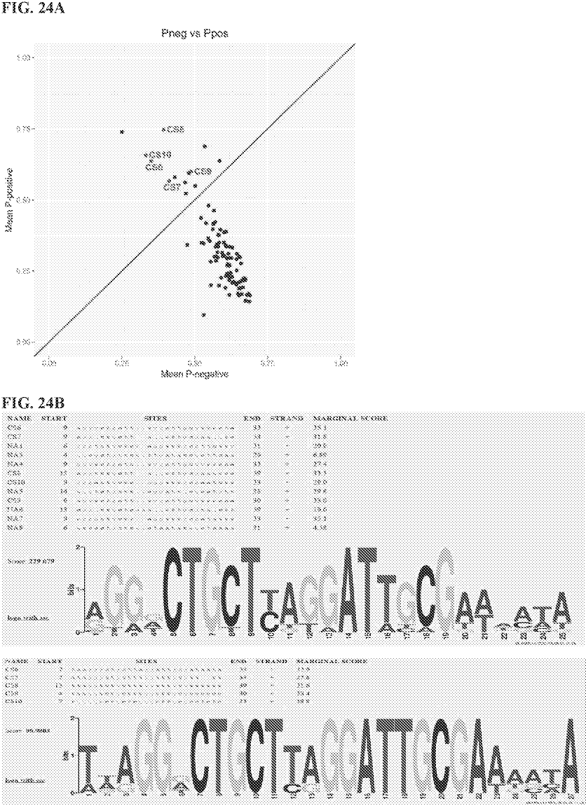

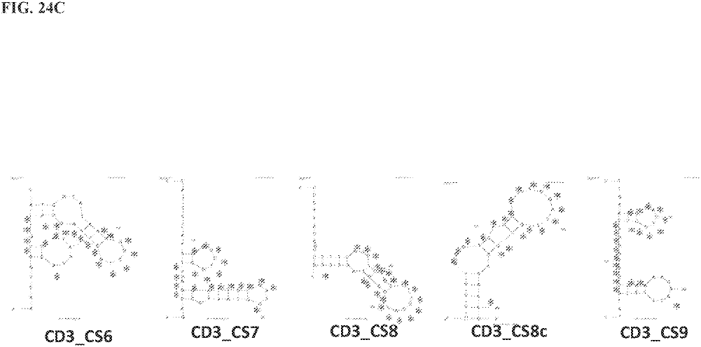

The mean P-positive and P-negative scores of the top 100 most abundant aptamers in the last round, were plotted (FIG. 24A), and aptamers with significant bound to unbound ratio as described above in #6 (p < 0.05; Poisson test, consistent in all rounds) were highlighted and selected for experimental validations (termed CD3-CS6-9, ID SEQ NO 88-91). The additional 9 aptamers with high mean P-positive values (P-positive > 0.5) were assigned an identifier (CD3_Ppos10-18 ID SEQ NO. 93-101)). The identified CD3 binding aptamers are listed in Table 18

TABLE 18

CD3-binding aptamers

| Aptamer name | SEQ ID NO: | Sequence 5′ to 3′ |
|---|---|---|
| CS6 | 88 | ATCGTATAAGGGCTGCTTAGGATTGCGATAATACGGTCAA |
| CS7 | 89 | CATTTCATAGGGCTGCTTAGGATTGCGAAGGTAATGCCAG |
| CS8 | 90 | CCCTTACCCCTTTTAGGTCTGCTTAGGATTGCGAAAAAAG |
| CS9 | 91 | TTGTAAGGACTGCTTAGGATTGCGAAAACAATATTCGTAT |
| CS8c | 92 | CTTTTAGGTCTGCTTAGGATTGCGAAAAAAG |
| Ppos 10 | 93 | TCCATGGGTCTGCTCTAGGATTGCGTTCATGGTCTCCCCG |
| Ppos 11 | 94 | AATTACAACCTTGGATTGCAAAGGGCTGCTGTGTTGTTTA |
| Ppos 12 | 95 | ATCGGAGCTGTTCCTTGATACCGATTCAAAAAGTTCGTAC |
| Ppos 13 | 96 | AATTTGTAGGGACTGCTCAGGATTGCGGATACAAATTAAT |
| Ppos 14 | 97 | AGACATTGGGGACTGCTCGGGATTGCGAATCTATGTCTCC |
| Ppos 15 | 98 | CCCTTTTTTAACTAGGTCTGCTTAGGATTGCGAATGTTAA |
| Ppos 16 | 99 | ACCTCAAAAGCGCGGGCTGCTCAAAGGATTGCGTAGCTTT |
| Ppos 17 | 100 | GGGGGTTAAGGGCTGCTTAGGATTGCGATAATACGGTCAA |
| Ppos 18 | 101 | AACATATAACTGCTCAATAATATAGATAAAATACTCACAA |

Next, the 14 aptamers with high mean P-positive values (P-positive > 0.5) (see Table 18) underwent multiple sequence alignment and a shared motif was found (FIG. 24B upper). In comparison, the highlighted candidates (CS6-9) were also aligned and a more robust motif was discovered (FIG. 24B bottom). In addition, structure prediction analysis was carried by analytic software (mfold, NUPACK) (FIG. 24C). This analysis demonstrated that candidates fold into a complex secondary structure mainly around the motif region. Following this result and in an optimization attempt, CD3_CS8 was further edited by trimming the first 9 nucleotides (denoted CD3_CS8cut) which seemed irrelevant to the formation of the secondary structure around the presented motif in CS_CD8. Top 5 candidates were further confirmed to possess a negative Delta G scores and were selected for individual binding assays.

In addition to the binding SELEX described above, a hybrid methodology was implemented, in which the process included also whole-cell SELEX rounds

TABLE 19

Alternative CD3- binding aptamers

| Aptamer name | SEQ ID NO: | Sequence 5′ to 3′ |
|---|---|---|
| CS1 | 102 | CTCTACCTGACTGTAACCTCTCGCTCCCCCCCATTCGCGC |
| CS2 | 103 | TTGTCCCTCTACGCCGCCCTTTACTACCACTCCTGCGATT |
| CS3 | 104 | TCCAGCACACCGACCGCCCCTCTACATTACCCCCTGGACT |
| CS4 | 105 | CCCCTCCATTCCCCCGCCTCGTCCACCCTACTCCTTAGTC |
| CS5 | 106 | CATCGACGCCCACACACCACTTCCCGTTCCCCTGCATCAT |

### Example 7 - Individual CD3 Binding Aptamers Validation (of Example 6)

### A. Aptamer Candidates Demonstrate Binding to Human CD3ε via HPLC

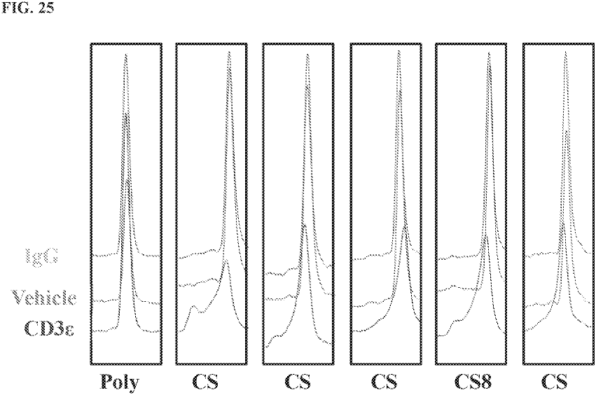

Top five candidates (CS6, CS7, CS8, CS9, and CS8c; SEQ ID NOs: 88-92, respectively) were synthesized with a 5(5′) phosphothioated CpG motif and assayed for Human CD3ε (hCD3ε) binding via the HPLC size exclusion column. In this method, the aptamers were labelled with Cy5 complementary sequence to the CpG site (Cy5-CpG’). Then, the folded-labelled candidates are incubated, each, with the CD3ε- recombinant protein or with negative control IgG1 (1 hr at 37℃ and 4℃) and analyzed by HPLC ProSEC 300S size exclusion column (Agilent) at 570 nm absorption. Upon protein binding, the aptamer-protein complex has a greater mass than a free aptamer and as a result, the retention time (RT) at the column is expected to be shorter. Inversely, in the case of non-binding aptamer, the RT in the presence of protein will be the same as in the absence of the protein. As a control, PolyT sequence was used. All five candidates demonstrated a binding to CD3 epsilon target protein at varying levels (FIG. 25)

### B. Aptamer Candidates Demonstrate Specific Binding to Jurkat T Cell Line And Primary Human Pan T Cell by Flow Cytometry

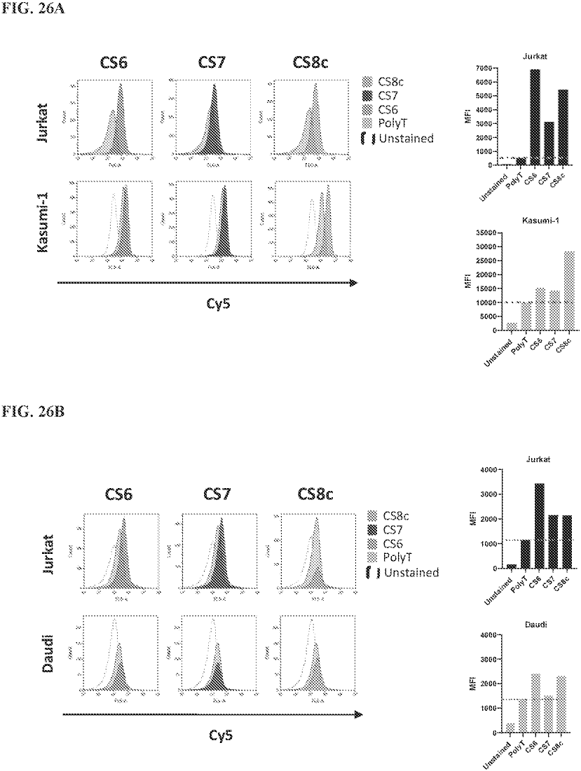

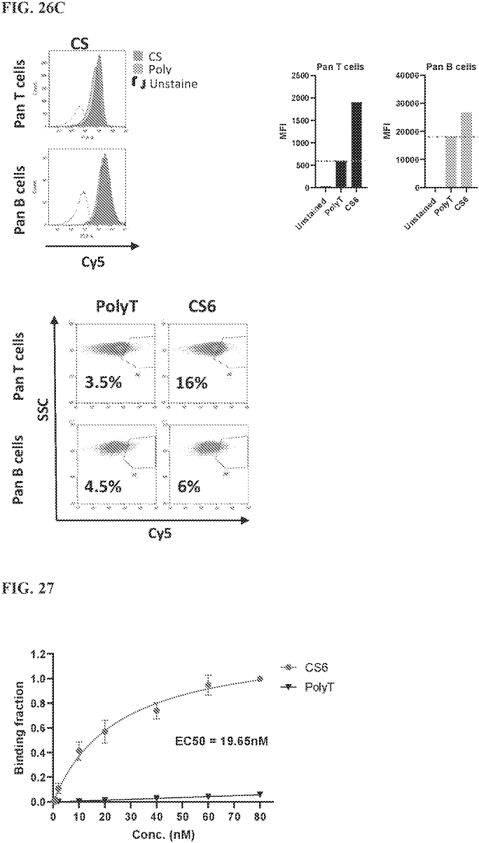

After CS6, CS7 and CS8c candidates demonstrated specific binding to CD3e recombinant protein, they were assayed for binding to their target in the native, whole -cell context, on the surface of T cells by flow cytometry. For this purpose, Jurkat T lymphocyte cell line (Acute T cell leukemia, ATCC TIB-152), previously reported to exhibit TCR expression, were used. The first binding assay with cells conducted at 4° C. for 1 hr. As a negative control, the myeloblast Kasumi-1 cell line was used (Acute myeloblastic leukemia, ATCC CRL-2724) All three candidates were found to differentially bind the target cells as compared with control cells while CS6 and CS7 demonstrated better specificity than CS8c. (FIG. 26A)

Next, to better mimic physiological conditions, the three candidates were assayed for binding Jurkat at 37° C. Here, as a negative control, B lymphoblast Daudi cell line was used (lymphoblast, ATCC CCL-213) (FIG. 26B). In this experiment, the three candidates bound the target cells when CS6 showed the highest binding level.

CS6 was selected for further exploring and characterization. It was found to bind normal primary Pan T cells and not Pan B cells at 37° C. under blocking conditions (FIG. 26C).

Subsequently CS6 effective concentration 50 (EC₅₀) was evaluated. A serial dilution of -Cy5 labelled aptamer was incubated with Jurkat cells for 1 hr at 37℃ and assessed for binding via flow cytometry (FIG. 27). The calculated EC₅₀ value was 19.65 nM.

Further, CS6 affinity towards CD3ε was tested by surface plasmon resonance (SPR) and its dissociation constant was calculated to be K_{d} = 31 nM (FIG. 28).

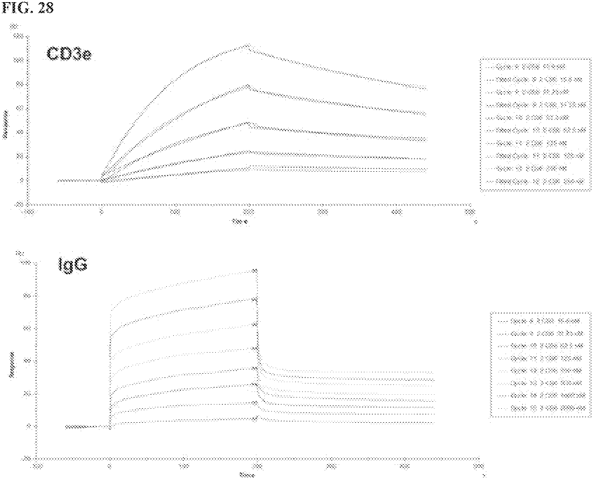

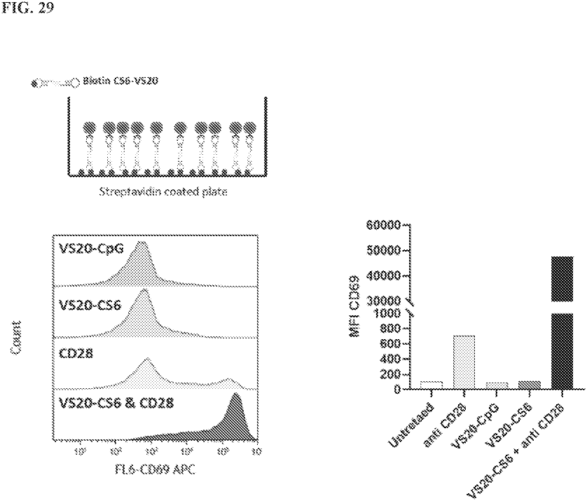

When hybridized to a Variable Strand exemplary sequence VS20 (SEQ ID NO.: 110) to form a bispecific T cell engager structure, CS6 has led to the stimulation of T cells, as demonstrated by elevation of CD69 markers (FIG. 29).

### Example 8 - TLR9 Agonistic Sequence Designed Into the Bispecific Personalized Aptamer Structure

### A. CpG Motif of the Bispecific Personalized Aptamer Modulate the Immune Response

TLR9 recently emerged as a potential therapeutic target for its ability to promote the presentation of tumorigenic antigens to adaptive immune cells and to stimulate the production of mediators with a direct antitumor activity. Class C CpG ODNs are potent inducers of IFN-α from plasmacytoid dendritic cell (pDC) and strong B cell activators (Marshall (2003), J Leukoc Biol 73(6):781-92) and in vivo studies have demonstrated that type C ODNs which combine the effects of types A and B ODNs, such as ODN 2395, are very potent Th1 adjuvant (Vollmer (2004) Eur. J. Immunol. 34, 251-262.)

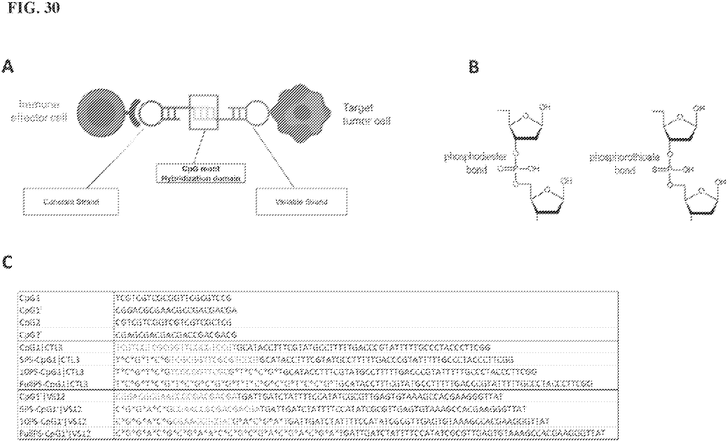

A novel CpG sequence was introduced into the bispecific personalized aptamer structure as a dimerization domain linking the two arms together (FIG. 30A). The dimerization sequence was 22 nt in length and rich in CpG dioligonucleotides (FIG. 30C).

It was first verified that the introduction of the new hybridization domain did not reduce target-lethality associated with the bispecific personalized aptamers’ primary mode of action. In a co-culture of PBMCs from healthy donors and HCT116 colorectal cancer cell line, the bispecific personalized aptamer was administered daily for 72 hrs, followed by a Live/Dead® dye and a flow cytometry analysis. No reduction in cytotoxic effect was observed using the new designed bispecific personalized aptamers and no significant differences were observed between the four tested CpG ODN-bearing bispecific personalized aptamers (FIG. 31A).

Since ODNs comprising phosphodiester backbones are degraded by nucleases, nuclease-resistant ODNs with phosphorothioate (PS) backbones have been developed (Eckstein (2014) Nucleic Acid Therapeutics 24:374-387; Pohar et al. (2017) Sci. Rep. 7). The replacement of the non-bridging oxygen with sulfur atoms (FIG. 30B) is a common chemical modification in the backbone of therapeutic oligonucleotides. and synthetic ODNs may consist of a partial or a complete phosphorothioate (PS) backbone for vaccine adjuvants and in cancer therapies (Pohar et al. (2017) Sci. Rep. 7). Next, four different variations of PS modifications were tested to rule out interference with the bispecific aptamer primary mode of action (sequences in FIG. 30C): (i) noPS — none of the 22nt comprising the dimerization domain was modified; (ii) 5PS — only the first five 5′ nucleotides of the dimerization domain were modified; (iii) 10PS - the first five nucleotides and the last five nucleotides of the dimerization domain were modified; (iv) 22PS - all 22nt comprising the dimerization domain were modified.

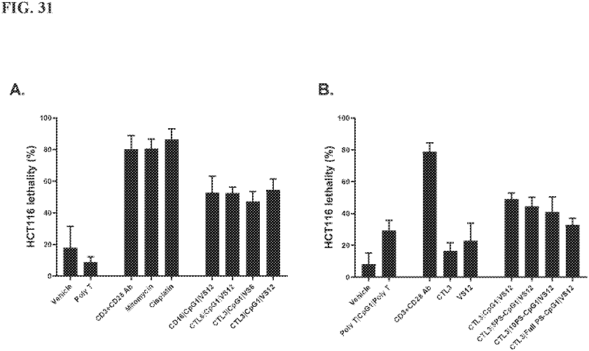

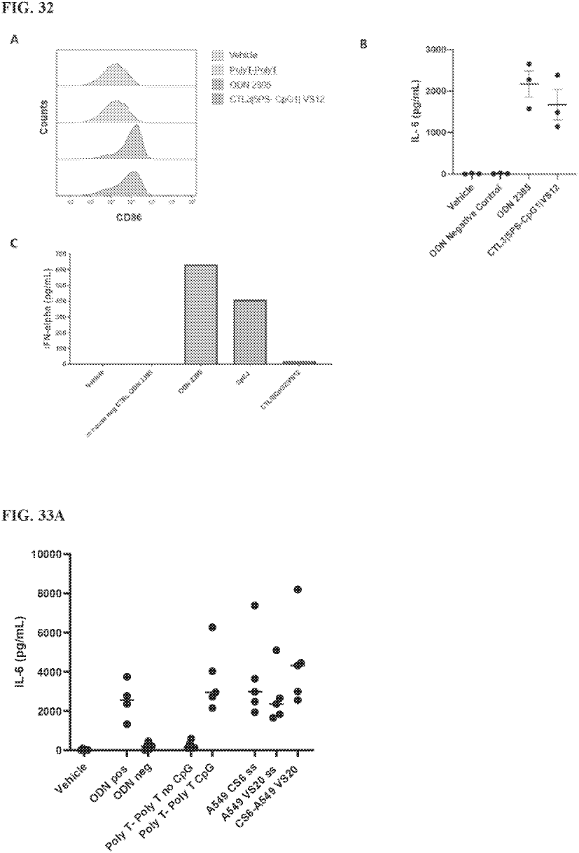

CTL3|CpG1|VS12 bispecific personalized aptamers with the different PS variants were examined for HCT116 cytotoxicity. As shown in FIG. 31B, full PS (i.e., 22PS) has demonstrated abrogated cytotoxicity. 5PS and 10PS on each monomer resulted in equivalent results, comparable to the initial bispecific personalized aptamer containing no PS, with the 10PS causing a slight decrease which was not significantly different. Hence, the 5PS modification has been selected for further studies. Two unique variants of the CpG bridge, CpG1 and CpG2, were generated and tested as TLR9 agonists (see FIG. 30C for specific sequences). Bispecific personalized aptamer CTL3|CpG1|VS12, in which the first five 5′ nucleotides of the dimerization domain were PS modified were tested for their immune-stimulation capacity. and compared with ODN2395 a canonical type C TLR9-activating oligo; (Roda et al. (2005) J. Immunol. 175:1619-1627; Abel et al. (2005) Clin. Diagn Lab Immunol. 12:606-621). Isolated human B cells were cultured with 50 µM CTL3|CpG1|VS12 bispecific aptamer, and the expression of the co-stimulation surface marker CD86 was assessed by flow cytometry. To rule out a non-specific effect induced by the presence of any DNA, a dimer of PolyT (50 µM), not containing the CpG motif was used as a control. Similar to the established TLR9 agonist ODN 2395, CTL3|CpG1|VS12 treatment has led to upregulation of CD86 on B cells (FIG. 32A). Splenocytes from BALB/c mice were isolated (n=3) and seeded in 96 wells plate (500,000 cells/well). Cells were treated with Vehicle, ODN negative control (5 µM), ODN 2395 (5 µM) as positive control and with bispecific aptamer CTL3|CpG1|VS12 (50 µM) for 48h. Forty-eight hrs post-treatment, cells were centrifuged and supernatant were collected and analyzed for IL-6 secretion using IL-6 ELISA kit (FIG. 32B). CpG2 sequence has also demonstrated TLR9 agonistic effect by inducing IFN-alpha secretion from PBMCs, yet, this function seemed to ne abrogated in the context of bispecific aptamer (FIG. 32C)

To ensure that the identity of the Constant Strand does not affect the previously introduced CpG function, a bispecific aptamer was formed using the CD3ε-targeting moiety CS6 (SEQ ID NO.: 116) and VS20 as the variable moiety (SEQ ID NO.: 110). IL-6 secretion was not affected by replacement of the aptameric Constant and Variable arms. Moreover, the CpG motif was active if the two arms were replaced with non-specific Poly T sequences.Interestingly, the CpG exerted a function even as a single strand DNA, albeit not as strong as in the double-strand structure (FIG. 33A). Additional data were generated to re-inforce the function of the novel CpG in driving antigen presentation and it was demonstrated to increase expression of CD86, CD80 and CD58 in human B cells (FIG. 33B). Titration plots for these markers were generated and demonstrated the bispecific aptamer, TLR9 agonistic activity EC₅₀ to be approximately 20 µM (FIG. 34).

### Example 9—Materials and Methods for Examples 10-12

### A. Materials

### A. Aptamers

Newly -identified, cancer-targeting tumoricidal aptamer arms were derived from a functional enrichment process as described in PCT Application No. PCT/IB19/01082 using the following target cells / organoids : HCT-116 colon carcinoma cell line (Variable Strands HCT116-VS6 and-VS12; SEQ ID NOs: 43 and 44, respectively), MCF7 breast cancer cells (MCF7-VS13, -VS16 and -VS19, SEQ ID NOs: 45, 46 and 47, respectively), A5449 adenocarcinomic human alveolar basal epithelial cells (A549-VS3 and VS20 , SEQ ID NOs: 107 and 108, respectively), colorectal carcinoma (CRC) - derived organoid #13 CRC-13 VS31, VS48 and VS81, SEQ ID NOs: 113-115, respectively).

T cell engager sequences (CTL3, CTL5 and CTL6, SEQ ID NOs: 3, 5, and 6, respectively) were derived from Cell-SELEX binding process as described in Examples 7-9. CD16 aptamer sequence was taken from the literature (Boltz et al. (2011) J. Biol. Chem. 286:21896-21905; Li et al. (2019)Molecules doi:10.3390/molecules24030478). CD3e-binding aptamer (CS6 SEQ ID NO: 88) were derived from a SELEX binding process, using human recombinant CD3e as described in Examples 10-11.

Aptamers were synthesized by standard solid phase synthesis on CPG resin, followed by either AEX column purification and ultrafiltration or standard desalting. Tumor-targeting, immune engager and CpG motif sequences are founds in Table 1. Table 20 below lists additional control and auxiliary sequences used in the different experiments.

TABLE 20

A List of control and auxiliary sequences

| Aptamer name | SEQ ID NO: | Sequence 5′ to 3′ |
|---|---|---|
| Non CpG 22b bridge | 67 | CTTAATCAGACATTATACAAAT |
| Non CpG 22b′ | 68 | ATTTGTATAATGTCTGATTAAG |
| Non CpG 18b bridge | 69 | GAATTAACAATTATAACG |
| Non CpG 18b′ | 70 | CGTTATAATTGTTAATTC |
| Non CpG 18b \|Poly T | 76 | GAATTAACAATTATAACGTTTTTTTTTTTTTTTTTTTTTTTTTTTTTTTTTTTTTTTTTTTTTTTTTTT |
| Non CpG 18b′ \|Poly T | 77 | CGTTATAATTGTTAATTCTTTTTTTTTTTTTTTTTTTTTTTTTTTTTTTTTTTTTTTTTTTTTTTTTTT |
| Non CpG \| Poly T | 78 | CTTAATCAGACATTATACAAATTTTTTTTTTTTTTTTTTTTTTTTTTTTTTTTTTTTTTTTTTTTTTTTTTTT |
| Non CpG′ \| Poly T | 79 | ATTTGTATAATGTCTGATTAAGTTTTTTTTTTTTTTTTTTTTTTTTTTTTTTTTTTTTTTTTTTTTTTTTTTTT |
| CpG1\| Poly T | 80 | TCGTCGTCGCGGTTCGCGTCCGTTTTTTTTTTTTTTTTTTTTTTTTTTTTTTTTTTTTTTTTTTTTTTTTTTTT |
| CpG1′ \| Poly T | 81 | CGGACGCGAACGCCGACGACGATTTTTTTTTTTTTTTTTTTTTTTTTTTTTTTTTTTTTTTTTTTTTTTT |

### B. Antibodies and Reagents

Leaf® purified anti-human CD3 and Leaf® purified anti-human CD28 antibodies, used for the stimulation of human PBMCs, and were purchased from Biolegend (ENCO). CD45-FITC antibody, used for leukocytes staining, was purchased from Miltenyi Biotec (Almog diagnostic). Mitomycin C, used as a positive control, was purchased from Sigma. Live/Dead® Fixable Violet Dead Cell Stain kit, for 405 nm excitation, was purchased from Thermo Fisher (Rhenium).

### C. Cell Lines and PBMCs Isolation

HCT-116 human colorectal cell line (ATCC® CCL-247®) were cultured in McCoy’s 5A supplemented with 10% fetal calf serum (FCS) and 1% Penicillin and streptomycin (Pen/Strep).

MCF10a non-tumorigenic cell line (ATCC® CRL-10317®) were cultured in DMEM/F12 supplemented with 5% horse serum, 1% Pen/Strep, 20 ng/ml EGF, 0.5 mg/ml Hydrocortisone, 100 ng/ml Cholera toxin and 10 µg/ml Insulin. All cells were cultured at 37° C. and 5% CO₂.

PBMCs were isolated by Ficoll density gradient centrifugation from peripheral blood from healthy donors (MDA Israel, Sheba hospital) using Lymphoprep® (Axis-Shield) following the manufacturer’s protocol. Isolated PBMCs were maintained in RPMI1640 from ATCC and supplemented with 10% fetal calf serum (FCS) and 1% Penicillin and streptomycin (Pen/Strep).

### D. Formulation Buffer / Vehicle

Phosphate-buffered saline (minus Magnesium and Calcium) supplemented with 1 mM Magnesium Chloride (MgCl₂). The folding buffer is sterilized with PVDF membrane filter unit 0.22 µm and kept at RT.

### E. Animals

Female NSG mice, 7-8 weeks’ old were purchased from Jackson Labs.

### B. Experimental Methods

### A. Bispecific Personalized Aptamer Formulation

Formulation procedure includes the following steps:
1. Reconstitution
   Each strand is diluted / reconstituted (if lyophilized) to the desired concentration in the formulation buffer.
2. Aptamer folding:
   a. Strands are heated for 5 minutes at 95° C.
   b. Rapid cooling for 10 minutes on ice.
   c. Incubation for 10 minutes at RT.
3. Bispecific entity formation

The two strands (cancer -targeting variable strand and the immune engager strand) are then mixed together and incubated in a rotator for 30 minutes at RT.

### B. Cytotoxicity Assay

HCT116 cells were seeded in a 96-wells plate 24 hours pre addition of PBMCs and treatments were added daily for 72 hours. Following the 72 hr treatment stage, cell media was removed and kept, while 30 µL trypsin was added to each well for 5 mins at 37° C. followed by 5 mins at 300xg spinning at 4° C. After centrifugation, cells were resuspended with 100 µl LIVE/DEAD® Fixable Violet Dead Cell Stain (Thermo Fisher) (1:1,000 in PBS) and incubated for 30 minutes on ice in the dark. Cells were washed once in washing buffer (PBS containing 1% BSA and 2 mM EDTA) and resuspended with 50 µL CD45-FITC antibody solution for on ice in the dark. The cells were washed once following analysis by flow cytometry.

### C. Gating Strategy

A Dead/Live® dye was used combined with the CD45 antibody staining for discriminating between immune cells and target HCT116 cells. The lethality of the target cells was determined by the percentage of cells stained positively for Live/Dead® dye.

### D. Animals

Female NSG® mice, 7-8 week old, were purchased from Jackson Labs. All animal procedures were performed in the facilities of Tel Aviv Sourasky medical center under ethical approval.

### E. Xenograft Models Induction and Interventions

### (I) HCT116 Early Intervention Model

Female NSG® mice were injected subcutaneous (SC) into the mouse right flank with 2x10⁶ HCT116 tumor cells admixed with 0.5x10⁶ fresh human PBMC in a 1:4 ratio with Cultrex® (Basement Membrane Matrix, Type 3), 0.2 ml/mouse. Regimen of SC interventions is detailed per experiment.

### (II) MCF7 Established Tumor Model

Female NSG® mice were injected SC into the mouse right flank with 2x10⁶ MCF7 tumor cells. Water with Estradiol was supplemented one week prior to MCF-7 implementation. When an established tumor was measured (50-100 mm³), 15x10⁶ fresh human PBMC were administered intravenously (IV). Four days post PBMCs injection, randomization was performed based on tumor volume and intratumoral (IT) interventions began, 3 times a week, for a total of 8 doses.

### F. Tumor Volume Method of Evaluation

Change in tumor volume was monitored by calipers three times per week. Tumor volume was estimated as follows: Tumor Volume (mm3) = length × width²/2

### G. Statistical Methods

All quantitative data are expressed as the mean ± SEM. Either ANOVA or Student t-test were used, when appropriate, in order to evaluate significance of difference between groups.

### Example 10 - Tumoricidal Aptamers Identified by Aummune’s Platform Were Found Efficacious in Vitro in Cancer Cell Lines and Tumor-Derived Organoids

Newly -identified, cancer-targeting tumoricidal aptamer arms were derived from a functional enrichment process as described in PCT Application No. PCT/IB19/01082.

In order to provide a proof-of-concept for Aummune’s platform ability to identify specific aptamer sequences to serve as the VS, HCT116 colon carcinoma cell line was used. These targeted cells, together with the negative control of human PBMCs from healthy donors as representative non-tumorigenic cells, were subjected to Aummune’s proprietary innovative aptamer selection platform and a potent and selective VS was isolated.

### A. Identifying the Functional Aptamer “Variable Strand 12” via Aummune’s SELEX Process

Aummune’s proprietary technology of functional SELEX was implemented using the human colon carcinoma cell line HCT116.

As per the funnel scheme describing the selection process (FIG. 2), the enrichment procedure has commenced with a random library of aptamers with a vast repertoire of 10¹⁵ individual sequences. As shown in FIG. 35, the aptamer populations indeed demonstrated a relative enrichment between rounds of enrichment with the eighth round of functional selection (F3.8) inducing 37.4% apoptotic cells, which is a 1.5-fold increase over the 25% apoptotic cells (the sample of clustered bead population) after the first round of functional selection (F3.1).

The DNA library underwent enrichment for apoptosis-inducing sequences in HCT116 cells during Functional Cell-SELEX. There was a 1.5-fold increase in Caspase 3/7 activation in Cycle 8 (F3.8) (37%) compared to Cycle 1 (F3.1) (25%).

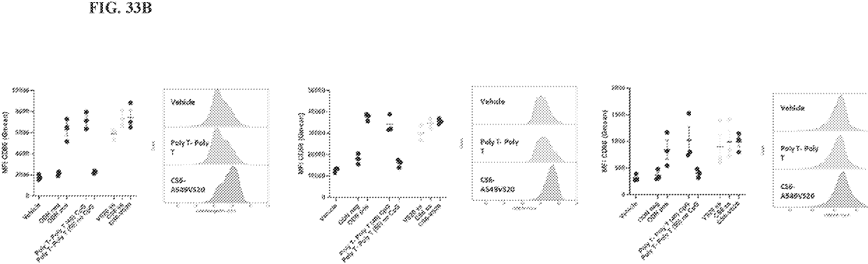

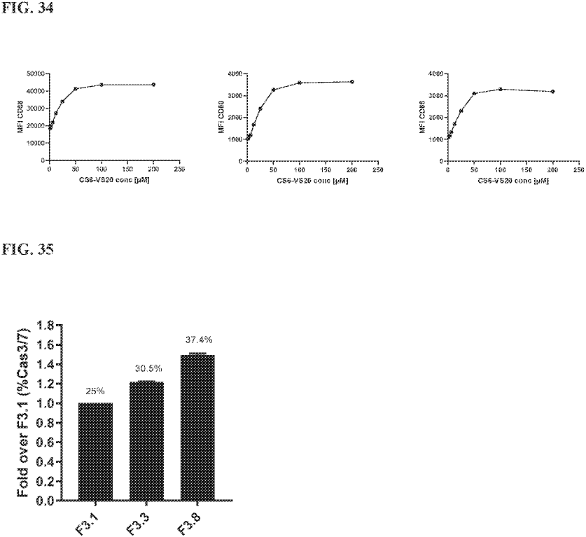

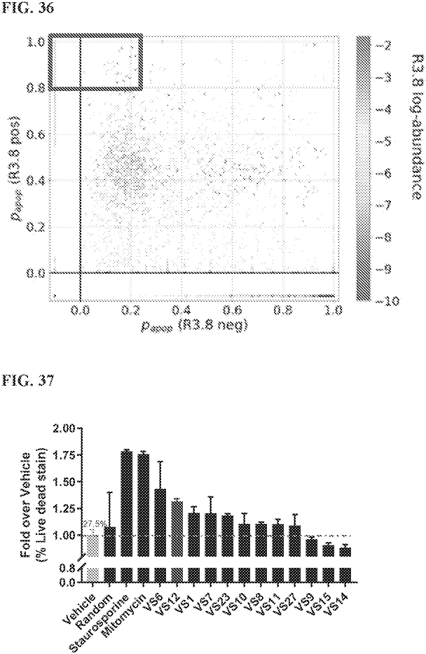

In the final round of functional selection, the clustered library was incubated with both target (“positive” HCT-116) cells and negative selection (“negative” PBMCs from a healthy donor). Positive and negative events were sorted from each cell population. Finally, libraries from final rounds for both target cells and negative cells were sequenced via NextSeq 500, followed by a bioinformatic analysis for each putative aptamer. Each aptamer was given two scores; one was the sequence propensity to induce apoptosis on the target cells (Y-axis, FIG. 36), and the second was the sequence propensity to induce apoptosis on the negative selection cells (X-axis, FIG. 36). The top 44 sequences, which had the highest Y-axis to X-axis score ratios, were screened individually via high-content fluorescence microscopy for their apoptosis-inducing ability.

The subsequent individual sequences screen was performed using high-content time-lapsed fluorescent microscopy. Target cells were incubated with a candidate aptamer for 24 hours and time-lapse imaging was applied to find putative sequences which successfully induced apoptosis on target cells.

Variable Strand 6 (VS6) and VS12 (SEQ ID NOs: 43 and 44, respectively) were selected to be further tested for their abilities to induce target cell death (FIG. 37), while VS12 was further assessed in a range of concentrations and displayed a dose-dependent cytotoxic effect. The data collectively show that VS12 was able to (i) induce Caspase activity, (ii) lead to increased target cell death as measured by flow cytometry, and (iii) substantially decrease viability of target cells.

### B. Identifying the Functional Aptamers Variable Strand 13 (VS13), VS16, and VS19 via Aummune’s SELEX Process

Functional Cell-SELEX was implemented using MCF7 human breast cancer cell line designed to obtain functional target-specific cytotoxic aptamers (using again process described herein as well as in PCT Application No: PCT/IB2019/001082).

During each round of the functional enrichment, the aptamer library was incubated with the target cell (MCF7) and stained with Annexin V as a cell death marker. PBMCs from a healthy donor were used for negative selection.

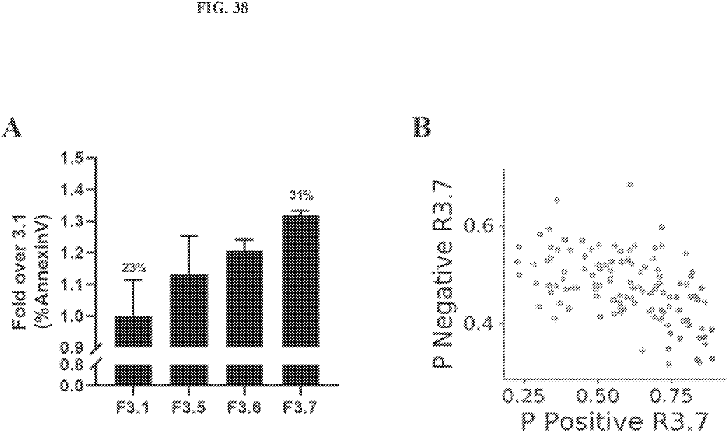

As shown in FIG. 38A, the aptamer library populations demonstrated a relative functional enrichment, increasing with each rounds of SELEX iteration. In the final round of Functional enrichment, the library was incubated with both target (“positive”; MCF7) cells and counter selection (“negative”; PBMCs from a healthy donor). Positive and negative events were sorted and sequenced. Each aptamer sequence was given two scores: (i) the sequence propensity to induce cell death on the target tumor cells (X-axis, FIG. 38B) and (ii) the sequence propensity to induce non-specific cell death on the counter selection PBMCs (Y-axis, FIG. 38B). Top 45 sequences which had the highest X-axis to Y-axis score ratio were screened individually via high-content fluorescence microscopy for their apoptosis-inducing ability.

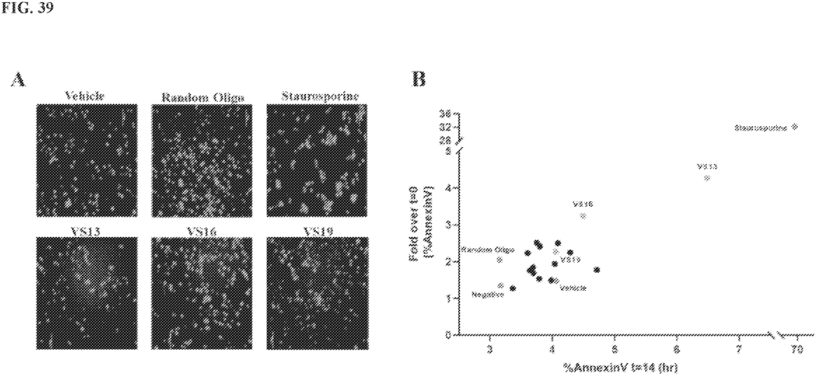

The subsequent individual sequences screen was performed using high-content time-lapsed fluorescence microscopy. MCF7 cells were cultured with a candidate aptamer for 24-hours and time-lapse imaging was applied to find putative sequences which successfully induced apoptosis on target cells. As negative controls, Vehicle (1xPBS-/-supplemented with 1 mM MgCl₂) and random sequences were used; as a positive control, Staurosporine was used. Three aptamer sequences, variable strands (VS13, VS16 and VS19), exhibited their ability to induce MCF7 cell death as individual aptamers (FIGS. 39A and 39B).

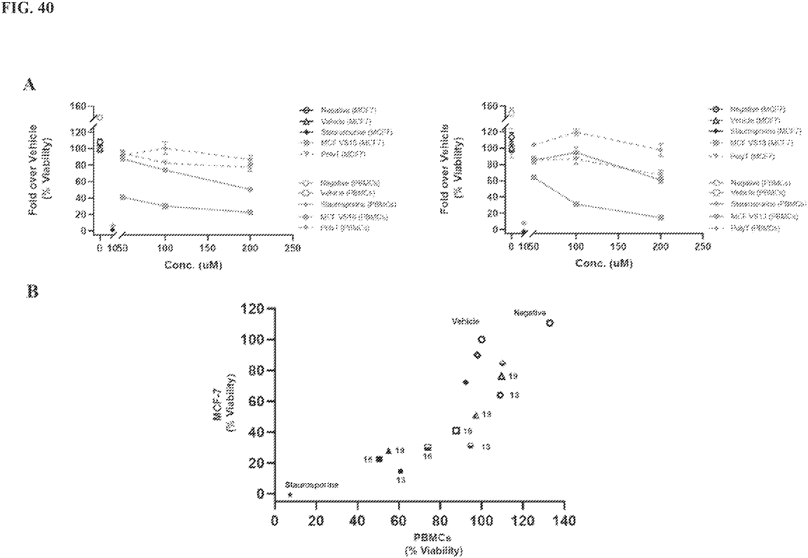

Top six candidates (VS4, VS11, VS13, VS16, VS19 and VS43) were further tested for their ability to affect MCF7 viability in a dose-dependent manner. The VS aptamers were concomitantly added to PBMCs culture to assess specificity of respective candidates. Viability of both MCF7 and PBMCs was determined using XTT assay. Culture with either VS13 or VS16 aptamers resulted in a significant decrease in viability of MCF7 target cells as compared with the non-specific, same-length, DNA sequence comprised of poly-thimidine nucleotides (PolyT)(FIG. 40A). VS13 and VS16 exhibited the desired features and fulfilled the criteria of promising VS candidates by inducing substantial cell death on the target cell population while having a minimal effect on the negative healthy PBMCs (FIGS. 40A and 40B).

Scatter plot summary shows MCF7 viability (Y-axis) versus PBMCs′ viability (X-axis) for lead aptamers tested (FIG. 40B) compared with the positive control (Staurosporine) and negative controls (Vehicle and Untreated). 6 lead aptamers and Poly T are indicated in hexagon for 200 µM dose, diamonds for 100 µM dose, and triangles for 50 µM dose level. VS13 and VS16 are indicated by “13” and “16” (FIG. 40B).

### C. Identifying the Functional Aptamers Variable Strand 3 (VS3) and VS20 via Aummune’s SELEX Process

Aummune’s proprietary technology was next implemented using the human adenocarcinomic alveolar basal epithelial lung cell, A549.

Similarly to HCT116 and MCF7, A549 functional round-to-round enrichment was demonstrated with the library of the eighth round of functional selection (F3.8) inducing 39% apoptotic cell death (FIG. 41), a 1.3-fold increase compared with 30% of apoptosis induction by the first round library pool (F3.1). As detailed in the two examples above, NGS sequencing followed by bioinformatic analysis of the final enriched library (F3.8) was performed and 90 individual aptamer sequences were further assessed by high-content microscopy.

Five top candidates (including VS3 and VS20) were assayed for their cytotoxic effects following a single dose administered at 50, 100, and 200 µM concentrations, culminating in measuring the cell viability ratio via the XTT assay (FIG. 42).

### D. Aummune’s SELEX Process Applied to Colorectal Cancer (CRC)-Derived Organoids

The robustness of the platform was demonstrated by providing data generated from a SELEX process performed on organoids derived and propagated from human primary tumor tissue.

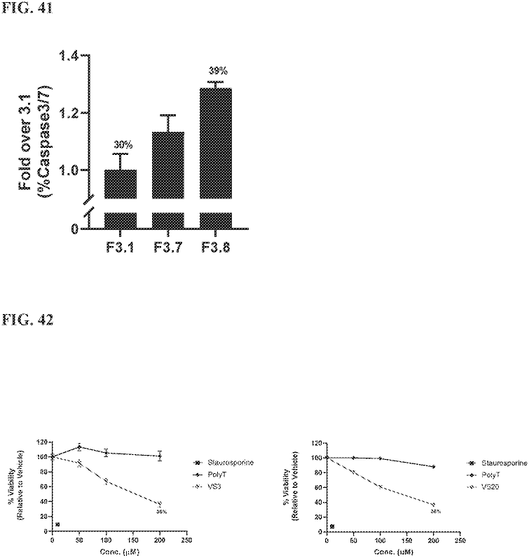

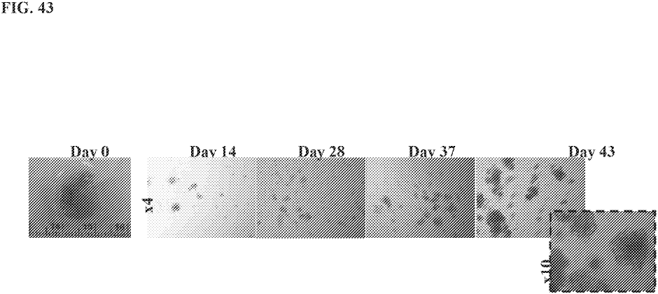

Fresh CRC tissue was removed from the patient during a surgical procedure, collected in a dedicated medium, and kept at 4° C. until processing. Next, the tissue underwent initialprocessing that combined mechanical and enzymatic dissociation with collagenase until fragments smaller than 0.1 mm were observed. The tissue fragments were mixed with a basement membrane extract (BME) and placed in an incubator to allow the BME to solidify. Then, CRC culture medium was added to the cells. After two weeks, a few organoid structures began to form and after three additional weeks, the number of organoids reached a critical mass for SELEX process initiation (FIG. 43).

As shown in FIG. 44A, the aptamer population pools showed relative functional increase with the seventh round of functional selection (F3.7) resulting in 31.8% of apoptotic cells, which is a 3.6-fold increase over the 8.7% of apoptotic cells observed with the second-round pool (F3.2).

In the final round of functional selection, the enriched library was incubated with both target cells and counter/ negative cell population (PBMCs from a healthy donor). Positive and negative events were sorted from each cell population. Enriched libraries from the final round, for both target cells and negative cells, were then sequenced via NextSeq 500 followed by a bioinformatics analysis in order to identify promising individual aptameric sequences. Sequencing data were analyzed via Aummune’s algorithm which allocated candidates for individual sequences functional confirmation. The algorithm utilized statistical estimators, tests, and metrics. Aummune has successfully implemented a high-content microscope screen for organoids in their assembled 3D configuration and within an extracellular supportive environment (BME) without having to dissociate the cells into a single-cell suspension. This setup enabled a long screen (up to 24 hours) and supported tumor cell viability over time. Aummune has calibrated the quantification of both active caspase and Annexin V using this assembled multi-cellular organoid method.

3 Variable Strands (VS31, VS48 and VS81, SEQ ID NOs: 113-115, respectively) identified by the abovementioned microscopy screen were tested individually for their abilities to induce tumor cell death using the CRC13 organoids as target and a luminescence-based viability assay. The Variable Strands were compared with a random sequence of 50% GC content (FIG. 44B).

### Example 77 - in Vitro Proof -of-Concept (POC) for Novel Bispecific Personalized Aptamers Efficacy

In some aspects, personalized cancer therapeutics described herein are composed of a heterodimeric structure with three separate domains (FIG. 1).

After a Functional Cell-SELEX designed to obtain functional apoptosis-inducing aptamers targeting HCT116 cell line (see Example 10a), two candidates were chosen (i.e., VS6 and VS12) to generate bispecific leads.

T-cell engagers, which were generated and characterized using a process described herein (see Example 3), were used as exemplars as the “constant” immune-engaging arms, in addition to a previously characterized CD16-binding Natural Killer (NK)-engager (Boltz et al. (2011) J. Biol. Chem. 286:21896-21905). Potentially other immune modulating aptamers can be also used (Soldevilla et al. (2016) Journal of Immunology Research 2016:1083738; Soldevilla et al. (2017) Immunotherapy - Myths, Reality, Ideas, Future doi:10.5772/66964).

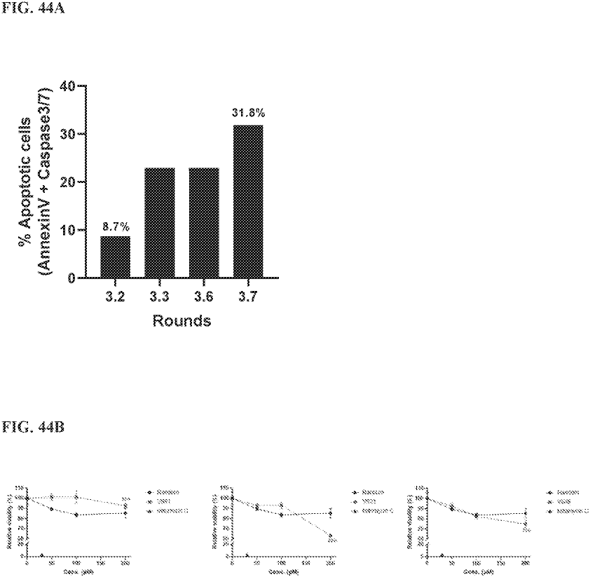

Five candidate bispecific personalized aptamers were generated (see FIG. 45) and listed in Table 21 below:

TABLE 21

Bispecific candidates

| | Immune effector cell engagers (T cell / NK cells) | HCT116 human colon carcinoma variable strands |
|---|---|---|
| 1 | CTL3 (T cells) | VS6 |
| 2 | CTL5 (T cells) | VS12 |
| 3 | CTL6 (T cells) | VS12 |
| 4 | CD16 aptamer (NK cells) | VS6 |
| 5 | CD16 aptamer (NK cells) | VS12 |

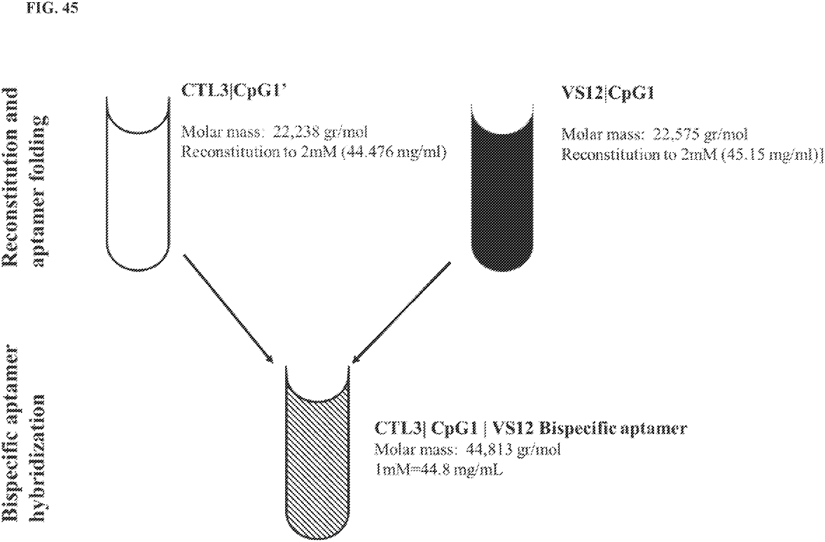

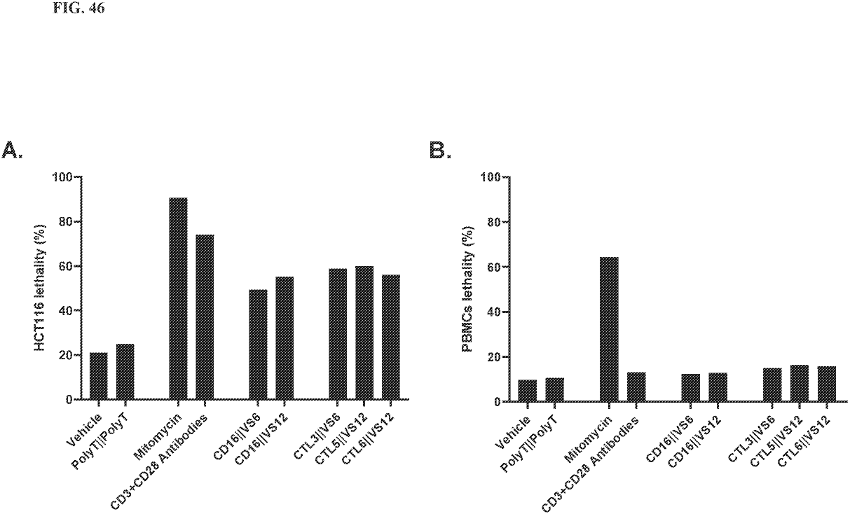

The NK and CTL bispecific personalized aptamers were assessed for their cytotoxic effects on HCT116 target cell line in a co-culture setting containing effector PBMCs from healthy donors, in an Effector-to-Target (E:T) ratio of 80:1. Unless otherwise specified, all treatments were administered daily, at 100 µM, for total duration of 72 hours (hrs). Tumor cell viability was subsequently analyzed by flow cytometry using LIVE/DEAD (Thermo Fisher) staining while gating on target cells only. Bispecifc aptamers were compared with the Vehicle negative control (1xPBS supplemented with 1 mM MgCl₂) and a non-specific DNA dimer comprised of two poly-thimidine (Poly T) arms, each of similar oligomer length as the bispecific strands. The results show high levels of lethality by all five bispecific personalized aptamers targeting HCT116 cells (~55%) and low effect on PBMCs (~17%), which is similar to the negative controls (10-12%) (FIGS. 46A and 46B). PBMCs lethality data reflects the specificity of bispecific personalized aptamers over mitomycin, a clinically approved chemotherapeutic drug which is highly promiscuous in its cytotoxic effect.

### A. Dose-Dependent Effect of Bispecific Personalized Aptamers Targeting HCT116 Cell Line

Next, the ability of bispecific personalized aptamers to target HCT116 cells in a dose-dependent manner was examined. Bispecific personalized aptamers CTL3||VS6, CTL5||VS12, CTL6||VS12 and control PolyT dimer (Poly T ||Poly T) were tested in four concentrations in a co-culture of PBMCs with HCT116 cells. Dose-dependency was exhibited for each of the tested bispecific personalized aptamers, but not with the negative control polyT||polyT dimer (FIG. 47).

### B. Bispecific Personalized Aptamers Are Target-Cell Specific in Their Cytotoxic Effect

MCF10a is a non-tumorigenic epithelial cell line used as a negative selection, along with PBMCs from healthy donors, during the Functional Cell-SELEX to identify VS12 aptamer and to increase the specificity of aptamers targeting HCT116 cell line (shown in PCT application no. PCT/IB2019/001082, incorporated herein by reference).

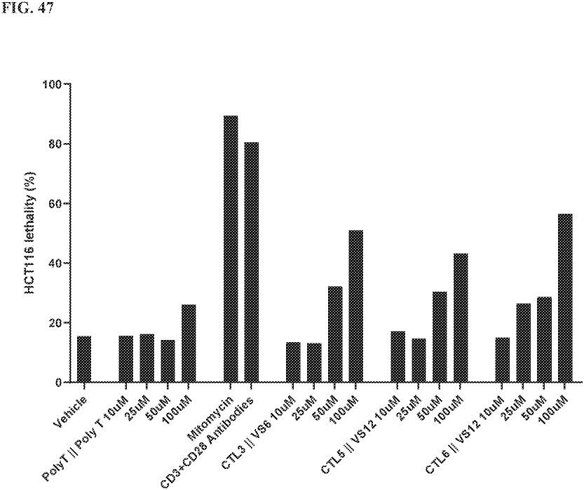

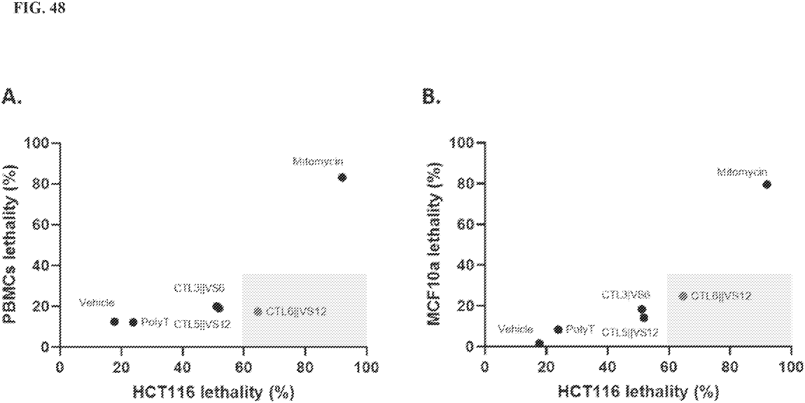

To demonstrate that the bispecific personalized aptamers attain selectivity while being potent towards the desired target, their ability to induce cell death was evaluated using PBMCs from healthy donors and MCF10a cells. (FIGS. 48A and 48B). CTL3 || VS12 displayed a favorable profile of >60% target-cell lethality and <30% off-target lethality (marked by a rectangle)

### C. Bispecific Personalized Aptamers Are Superior to the Cancer-Targeting Aptamer Moiety Alone

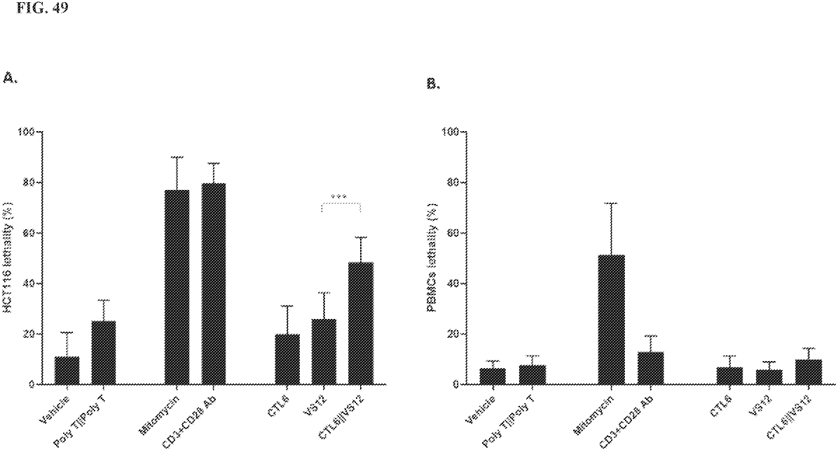

The target-cytotoxic potency of the bispecific personalized aptamer was compared with that of either monomers alone. Either CTL6||VS12 bispecific personalized aptamer, or one of its monomer strands were tested, each, for their ability to induce HCT116 tumor cell death at equivalent concentrations of 100 µM. CTL6||VS12 bispecific personalized aptamer was significantly superior to either monomer as well as to the polyT||polyT negative control (FIG. 49A). Both bispecific personalized aptamer and monomers did not induce PBMCs lethality (FIG. 49B).

### D. CTL3||VS12 and CTL6||VS12 Induced a Similar Cytotoxic Effect

An additional promising bispecific personalized aptamer lead CTL3||VS12, which was not previously tested was compared alongside with CTL6||VS12 for its cytotoxic effect on HCT-116 target cells in a co-culture assay with PBMCs. Both bispecific personalized aptamers proved to demonstrate similar cytotoxic effects on the target cells (rectangle FIG. 50) which was significantly higher than either monomer alone (FIG. 50).

### E. POC of CD3-targeting Bispecific Aptamer Conjugate

VS12 was hybridized to the T cell engager moiety (the CS) to form the bispecific, dual-acting aptamer CS6-VS12. CS6-VS12 Bispecific Aptamer was assessed for its ability to induce target cell cytotoxicity.

CS6-VS12 was tested for a cytotoxic effect on the HCT116 colon carcinoma cell line in a co-culture setting containing effector PBMCs from healthy donors in an Effector-to-Target (E:T) ratio of 10:1. Tumor cell viability was subsequently analyzed by luminescence-based cell viability assay. CS6-VS12 was compared with the Vehicle negative control (1 x PBS supplemented with 1 mM MgCl₂) and a non-specific DNA dimer comprised of two poly-thimidine (PolyT) arms, each of similar oligomer length as the bispecific strands (FIG. 51).

### F. Bispecific Aptamer Targeting MCF7 Breast Cancer Cells

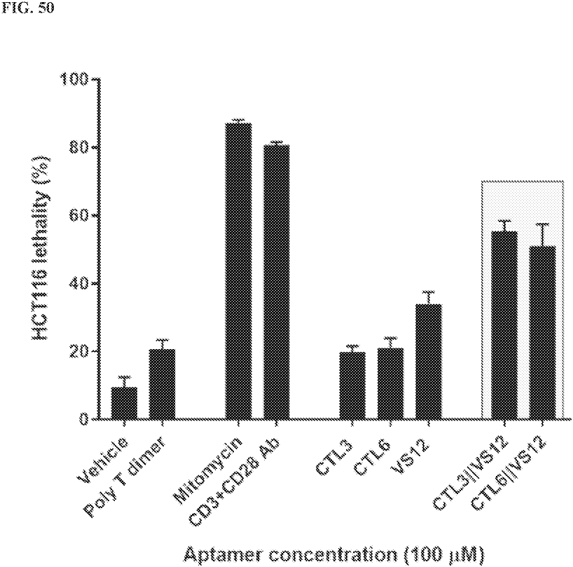

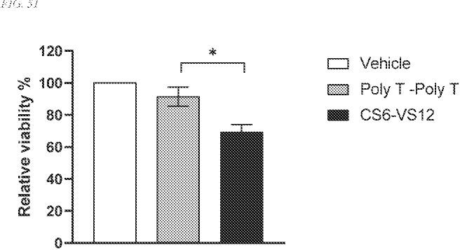

CTL3 comprising the T cell engager moiety of the bispecific aptamer, stemmed from a selection process targeting human CD8 T cells, performed with multiple donors, and its characterization is detailed in the Examples 2-4. VS13, VS16 and VS19 were each hybridized to CTL3 to form bispecific aptamers. These VS-CTL3 Bispecific Aptamers were assessed for their cytotoxic effect on MCF7 target cells in a co-culture setting with PBMCs from healthy donors. Tumor cells lethality was subsequently analyzed by flow cytometry and to have the complementary information, viability by XTT. Bispecific aptamers (CTL3||VS13, CTL3||VS16 and CTL3||VS19) were compared with Vehicle and a dimer comprised of two PolyT arms. . All three bispecific entities were found to have a significant cytotoxic activity in comparison with the Vehicle and PolyT controls (FIGS. 52A and 52B).

### Example 12 - in Vivo POC of Bispecific Personalized Aptamers in HCT116 and MCF7 Tumor Xenograft Model

The in vitro validated Bispecific Personalized Aptamer was tested for its ability to destroy target tumor cells in an in vivo setting.

### A. NK Cell Engager CD16 || VS12 in Vivo Efficacy

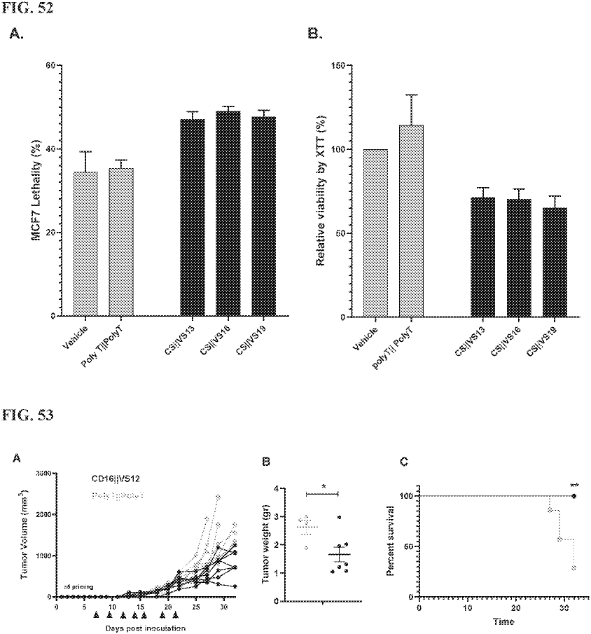

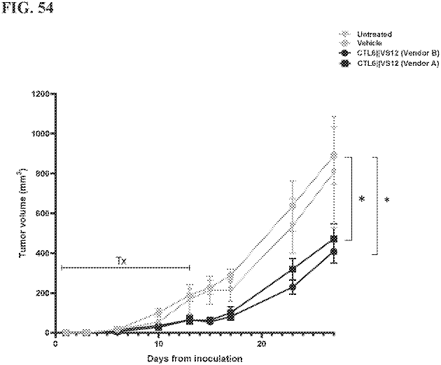

Female NSG® mice were injected SC into the mouse right flank with 2x10⁶ HCT116 tumor cells admixed with 0.5x10⁶ fresh human PBMC in a 1:4 ratio with Cultrex® (Basement Membrane Matrix, Type 3), 0.2 ml/mouse and treated either with NK engager CD16||VS12 or with the polyT dimer (PolyT||PolyT) as control. FIG. 53 shows the efficacy of the treatment compared to PolyT administration after 12 interventions during a 32-day study. All 7 treated mice showed inhibition in tumor growth compared to the polyT. Further, CD16||VS12 associated tumor growth attenuation has conferred a better survival rate.

### B. T Cell Engager CTL6||VS12 in Vivo Efficacy

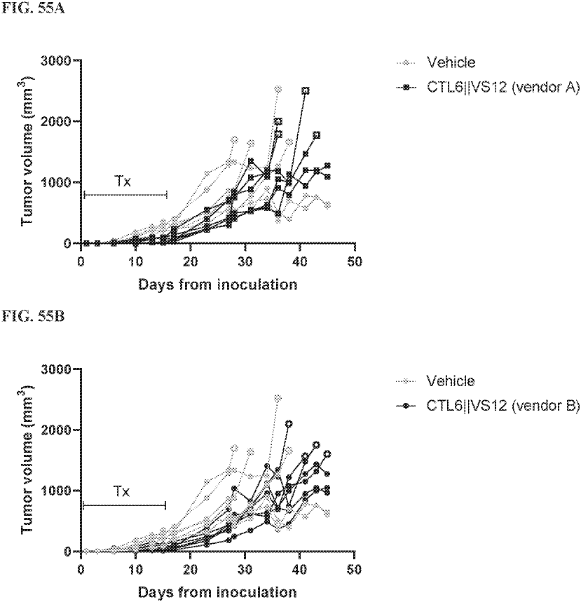

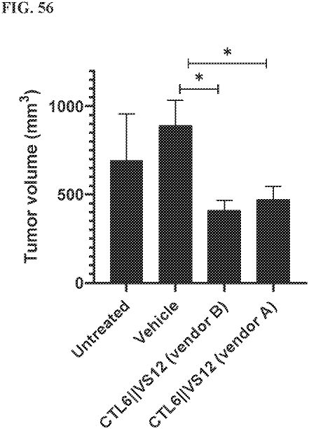

As above, female NSG® mice were inoculated with 2x10⁶ HCT116 tumor cells admixed with 0.5x10⁶ fresh human PBMC in a 1:4 ratio and treated with either Vehicle, CTL6||VS12 from vendor A or CTL6||VS12 synthesized by vendor B. While vendor A provided the aptamers without any modifications and with a purification method of standard desalting, vendor B provided the aptamer with inverted dT in both the 3′ and 5′ flanks and as a product of column purification. FIG. 54 shows the efficacy of the treatment compared to vehicle and untreated groups after 10 interventions during the first 27 days of the study (following Day 27, mice began to be scarified due to ethical volume for endpoint). Both groups of CTL6||VS12-treated mice demonstrated significant inhibition in tumor growth. Comparing tumor volume on Day 27 showed significant difference with both bispecific personalized aptamers (FIG. 56) compared to Vehicle. Individual mice tumor volume is presented till the end of the study (30 days after last intervention) for each bispecific personalized aptamer treatment compared to vehicle (FIGS. 55A and 55B).

### C. T Cell Engager CTL3||VS12 in Vivo Efficacy

HCT116 colon carcinoma cells were co-implanted with fresh human PBMC from healthy donors in an immune-deficient female NOD scid gamma (NSG®) mice, followed by administration of Vehicle, PolyT dimer or CTL3||VS12 as detailed in Table 22.

TABLE 22

in vivo treatments schedule

| Treatment | Dose (mg/kg) | Route of Administration | Number of interventions | Days of treatment |
|---|---|---|---|---|
| Untreated | N/A | N/A | N/A | N/A |
| Vehicle | N/A | SC | 10 | 0,1,2,3,4,6,7,8,9,10 |
| PolyT\|\|PolyT | 100 | SC | 10 | 0,1,2,3,4,6,7,8,9,10 |
| CTL3\|\|VS12 | 100 | SC | 10 | 0,1,2,3,4,6,7,8,9,10 |

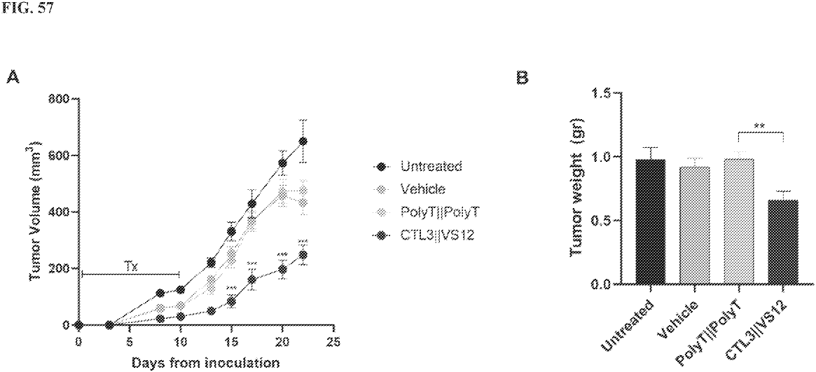

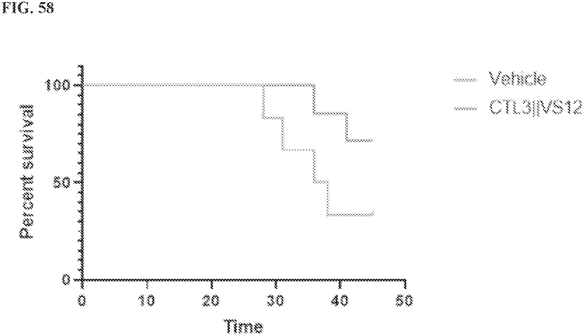

FIGS. 56A and 56B describe HCT116 tumor growth kinetics. Treatment with the Bispecific Aptamer CTL3||VS12 but not with the non-specific PolyT||PolyT dimer, has significantly attenuated the growth of HCT116 tumors (FIG. 57A), resulting in an average tumor size which is approximately 30% smaller, in weight, than the control groups on Day 22 (FIG. 57B). FIG. 58 depicts the survival curve of this experiment, suggesting a benefit for the treated group.

### D. CS6-VS12 Bispecific Aptamer Attenuates Tumor Growth In Vivo

In the xenograft model, HCT116 colon carcinoma cells were co-implanted with fresh human PBMC from healthy donors in an admix manner (E:T 1:4 ratio), in immune-deficient female NSG mice, and were administered with Bispecific Personalized Aptamer (CS6-VS12, SEQ ID NOs: 116 and 50), PolyT duplex, or vehicle.

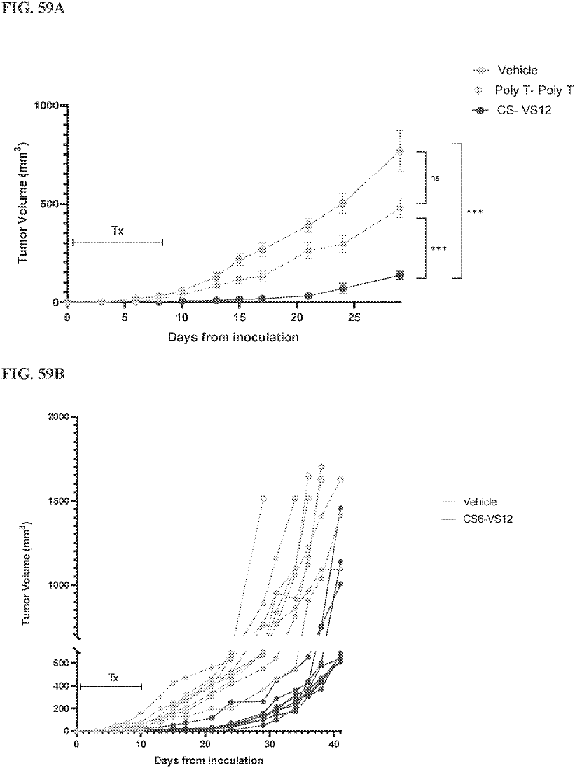

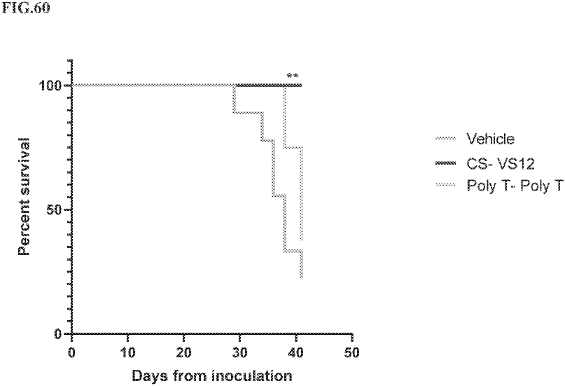

FIGS. 59A and 59B describe HCT116 tumor growth kinetics. Treatment with the Bispecific aptamer CS6-VS12, but not with the non-specific oligonucleotide PolyT, significantly attenuated the growth of HCT116 tumors after a total of 10 interventions. As of Day 30, mice began to be scarified due to ethical volume for endpoint. Individual mice tumor volume were presented until Day 41 (31 days after last intervention). Inhibition in tumor growth was demonstrated in all CS6-VS12 treated mice (FIG. 59B). Tumor growth reduction was translated to a benefit in survival for the bispecific-treated group, as compared to Vehicle (FIG. 60).

### E. MCF7-Targeting Bispecific Aptamer CTL3||VS16 Efficacy in Vivo, in An Established Tumor Model

The translatability of CTL3||VS16 cytotoxic effect from in vitro setting to in vivo, was assessed in an established MCF7 tumor xenograft model.

TABLE 23

in vivo treatments schedule

| Group | Intervention | ROA | Dose (mg/mouse) | Regiment |
|---|---|---|---|---|
| 1 | Untreated | N/A | N/A | N/A |
| 2 | Vehicle | IT | Equal Vol | Q3W |
| 3 | CTL3-VS16 | IT | 1.8 mg | Q3W |

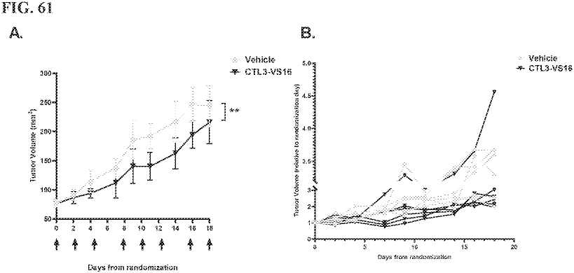

A significant inhibition in tumor growth was demonstrated in CTL3-VS16 treated mice, compared with Vehicle treated mice (FIGS. 61A and 61B).

### F. Murine 4T1-Targeting Bispecific Aptamer CS6-VS32 Efficacy in Vivo, In Combination with Immune Checkpoint Inhibitor

In order to enable efficacy in vivo animals in immunocompetent animals (in addition to the above-mentioned xenograft models), the murine breast cancer cell line 4T1 was subjected to the functional enrichment platform (similarly to other examples in Example 10) and VS32 was identified. VS32 was hybridized to CS6 to form the bispecific aptamer and was assessed in a dual-flank 4T1 tumor model.

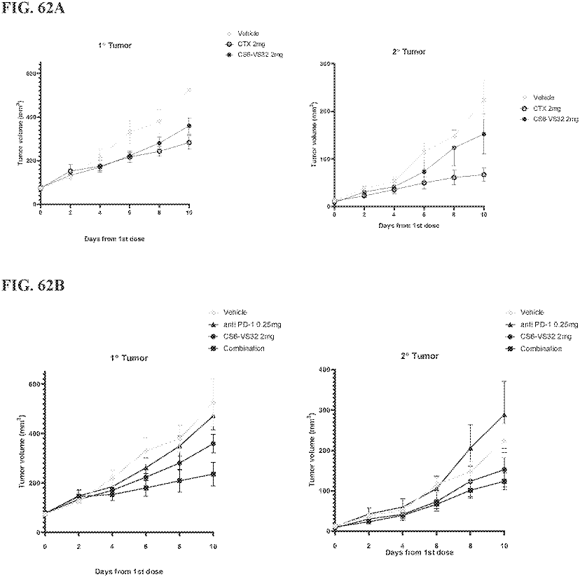

A trend of hindered growth of both the primary and secondary tumors was demonstrated by intratumoral administration of CS6-VS32 into the primary established tumor (FIG. 62A). Cyclophosphamide (CTX) chemotherapy was used as a positive control, in an equivalent dose.

When administration of CS6-VS32 was combined with the immune checkpoint inhibitor anti-PD1, a synergistic effect was demonstrated, leading to a significant tumor growth reduction, both at the injected tumor and in the secondary, non-injected one (FIG. 62B).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A bispecific personalized aptamer comprising:
(a) a cancer cell-binding strand that specifically binds to an antigen expressed on a cancer cell;
(b) a CpG motif sequence; and
(c) an immune effector cell-binding strand that specifically binds to an antigen expressed by an immune effector cell,
wherein the cancer cell-binding strand is linked to the immune effector cell-binding strand by the CpG motif sequence.

2. The bispecific personalized aptamer of claim 1, wherein the cancer cell-binding strand induces cell death when contacted to a cancer cell.

3. The bispecific personalized aptamer of claim 1 or 2, wherein the cell death is apoptosis, necrosis, immunological cell death, autophagy or necroptosis.

4. The bispecific personalized aptamer of any one of claims 1-3, wherein the cancer cell is a patient-derived cancer cell.

5. The bispecific personalized aptamer of any one of claims 1-4, wherein the cancer cell is a solid tumor cell.

6. The bispecific personalized aptamer of claim 5, wherein the cancer cell is a carcinoma cell.

7. The bispecific personalized aptamer of claim 6, wherein the carcinoma cell is a breast cancer cell, a head and neck cancer cell, a bladder cancer cell, or a colorectal carcinoma cell.

8. The bispecific personalized aptamer of any one of claims 1-4, wherein the cancer cell is a sarcoma cell.

9. The bispecific personalized aptamer of any one of claims 1-4, wherein the cancer cell is a hematologic cancer cell.

10. The bispecific personalized aptamer of any one of claims 1-9, wherein the cancer cell-binding strand induces cell death when contacted to the cancer cell in vitro.

11. The bispecific personalized aptamer of any one of claims 1-10, wherein the cancer cell-binding strand induces cell death when contacted to the cancer cell in vivo.

12. The bispecific personalized aptamer of any one of claims 1-11, wherein the immune effector cell-binding strand mediates lysis of the cancer cell through T cell or NK cell-mediated cytotoxicity.

13. The bispecific personalized aptamer of any one of claims 1-12, wherein the cancer cell-binding strand and the immune effector cell-binding strand are linked together by hybridization of a 5′ sequence of the cancer cell-binding strand to a 5′ sequence of the immune effector cell-binding strand.

14. The bispecific personalized aptamer of any one of claims 1-13, wherein the 5′ sequence of the cancer cell-binding strand hybridizes to the 5′ sequence of the immune effector cell-binding strand to form the double-stranded CpG motif sequence.

15. The bispecific personalized aptamer of claim 14, wherein the CpG motif sequence acts as a TLR agonist, and induces TLR9-mediated antigen presenting cell (APCs) stimulation and/or increased uptake of tumor antigens.

16. The bispecific personalized aptamer of any one of claims 1-15, wherein the CpG motif sequence induces an anti-tumor immune response.

17. The bispecific personalized aptamer of any one of claims 1-16, wherein the CpG motif sequence induces IL6 secretion, IFNα secretion, and/or B-cell activation.

18. The bispecific personalized aptamer of any one of claims 1-17, wherein the CpG motif sequence is a double-stranded nucleic acid sequence comprising a sequence that is at least 80% identical to any one of SEQ ID NOs: 63-66.

19. The bispecific personalized aptamer of any one of claims 1-18, wherein the CpG motif sequence is a double-stranded nucleic acid sequence comprising a sequence that is at least 90% identical to any one of SEQ ID NOs: 63-66.

20. The bispecific personalized aptamer of any one of claims 1-19, wherein the CpG motif sequence is a double-stranded nucleic acid sequence comprising a sequence that is at least 95% identical to any one of SEQ ID NOs: 63-66.

21. The bispecific personalized aptamer of any one of claims 1-20, wherein the CpG motif sequence is a double-stranded nucleic acid sequence comprising a sequence that is at least 98% identical to any one of SEQ ID NOs: 63-66.

22. The bispecific personalized aptamer of any one of claims 1-21, wherein the CpG motif sequence is a double-stranded nucleic acid sequence comprising a sequence of any one of SEQ ID NOs: 63-66, optionally wherein the CpG motif sequence is a double-stranded nucleic acid sequence comprising a sequence of SEQ ID NOs: 63 and 64.

23. The bispecific personalized aptamer of any one of claims 1-22, wherein the CpG motif sequence is a double-stranded nucleic acid sequence comprising at least 15 consecutive nucleotides of any one of SEQ ID NO: 63-66.

24. The bispecific personalized aptamer of any one of claims 1-23, wherein the CpG motif sequence has a length of no more than 30 nucleotides.

25. The bispecific personalized aptamer of any one of claims 1-24, wherein the cancer cell-binding strand binds to a cancer antigen selected from Prostate Membrane Antigen (PSMA), Cancer antigen 15-3 (CA-15-3), Carcinoembryonic antigen (CEA), Cancer antigen 125 (CA-125), Tyrosinase, gp100, MART-⅟melan-A, HSP70-2-m, HLA-A2-R17OJ, HPV16-E7, MUC-1, HER-2/neu, Mammaglobin-A or MHC-TAA peptide complexes.

26. The bispecific personalized aptamer of any one of claims 1-25, wherein the cancer cell-binding strand comprises a nucleic acid sequence that is at least 80% identical to any one of SEQ ID NOs: 43-62 or 107-115.

27. The bispecific personalized aptamer of any one of claims 1-26, wherein the cancer cell-binding strand comprises a nucleic acid sequence that is at least 90% identical to any one of SEQ ID NOs: 43-62 or 107-115.

28. The bispecific personalized aptamer of any one of claims 1-27, wherein the cancer cell-binding strand comprises a nucleic acid sequence that is at least 95% identical to any one of SEQ ID NOs: 43-62 or 107-115.

29. The bispecific personalized aptamer of any one of claims 1-28, wherein the cancer cell-binding strand comprises a nucleic acid sequence that is at least 98% identical to any one of SEQ ID NOs: 43-62 or 107-115.

30. The bispecific personalized aptamer of any one of claims 1-29, wherein the cancer cell-binding strand comprises a nucleic acid sequence of any one of SEQ ID NOs: 43-62 or 107-115.

31. The bispecific personalized aptamer of any one of claims 1-30, wherein the cancer cell-binding strand comprises at least 30 consecutive nucleotides of any one of SEQ ID NOs: 43-62 or 107-115.

32. The bispecific personalized aptamer of any one of claims 1-31, wherein the cancer cell-binding strand comprises at least 40 consecutive nucleotides of any one of SEQ ID NOs: 43-62 or 107-115.

33. The bispecific personalized aptamer of any one of claims 1-32, wherein the cancer cell-binding strand comprises at least 50 consecutive nucleotides of any one of SEQ ID NOs: 43-62 or 107-115.

34. The bispecific personalized aptamer of any one of claims 1-33, wherein the cancer cell-binding strand comprises at least 60 consecutive nucleotides of any one of SEQ ID NOs: 43-62 or 107-115.

35. The bispecific personalized aptamer of any one of claims 1-34, wherein the cancer cell-binding strand is no more than 120 nucleotides in length.

36. The bispecific personalized aptamer of any one of claims 1-35, wherein the cancer cell-binding strand is no more than 90 nucleotides in length.

37. The bispecific personalized aptamer of any one of claims 1-36, wherein the cancer cell-binding strand is no more than 80 nucleotides in length.

38. The bispecific personalized aptamer of any one of claims 1-37, wherein the cancer cell-binding strand is no more than 63 nucleotides in length, optionally wherein the cancer cell-binding strand is 63 nucleotides in length.

39. The bispecific personalized aptamer of any one of claims 1-38, wherein the immune effector cell-binding strand binds to an antigen expressed by T cells, NK cells, B cells, macrophages, dendritic cells, neutrophils, Basophils or Eosinophils.

40. The bispecific personalized aptamer of any one of claims 1-39, wherein the immune effector cell-binding strand binds to an immune effector cell antigen selected from CD16, Notch-2, other Notch family members, KCNK17, CD3, CD28, 4-1BB, CTLA-4, ICOS, CD40L, PD-1, OX40, LFA-1, CD27, PARP16, IGSF9, SLC15A3, WRB and GALR2.

41. The bispecific personalized aptamer of any one of claims 1-40, wherein the immune effector cell-binding strand comprises a nucleic acid sequence that is at least 80% identical to any one of SEQ ID NOs: 1-42, 88-106 or 116.

42. The bispecific personalized aptamer of any one of claims 1-41, wherein the immune effector cell-binding strand comprises a nucleic acid sequence that is at least 90% identical to any one of SEQ ID NOs: 1-42, 88-106 or 116.

43. The bispecific personalized aptamer of any one of claims 1-42, wherein the immune effector cell-binding strand comprises a nucleic acid sequence that is at least 95% identical to any one of SEQ ID NOs: 1-42, 88-106 or 116.

44. The bispecific personalized aptamer of any one of claims 1-43, wherein the immune effector cell-binding strand comprises a nucleic acid sequence that is at least 98% identical to any one of SEQ ID NOs: 1-42, 88-106 or 116.

45. The bispecific personalized aptamer of any one of claims 1-44, wherein the immune effector cell-binding strand comprises a nucleic acid sequence of any one of SEQ ID NOs: 1-42, 88-106 or 116.

46. The bispecific personalized aptamer of any one of claims 1-45, wherein the immune effector cell-binding strand comprises at least 20 consecutive nucleotides of any one of SEQ ID NOs: 1-42, 88-106 or 116.

47. The bispecific personalized aptamer of any one of claims 1-46, wherein the immune effector cell-binding strand comprises at least 30 consecutive nucleotides of any one of SEQ ID NOs: 1-42, 88-106 or 116.

48. The bispecific personalized aptamer of any one of claims 1-47, wherein the immune effector cell-binding strand comprises at least 40 consecutive nucleotides of any one of SEQ ID NOs: 1-42, 88-106 or 116.

49. The bispecific personalized aptamer of any one of claims 1-48, wherein the immune effector cell-binding strand comprises at least 50 consecutive nucleotides of any one of SEQ ID NOs: 1-42, 88-106 or 116.

50. The bispecific personalized aptamer of any one of claims 1-49, wherein the immune effector cell-binding strand is no more than 120 nucleotides in length.

51. The bispecific personalized aptamer of any one of claims 1-50, wherein the immune effector cell-binding strand is no more than 90 nucleotides in length.

52. The bispecific personalized aptamer of any one of claims 1-51, wherein the immune effector cell-binding strand is no more than 80 nucleotides in length.

53. The bispecific personalized aptamer of any one of claims 1-52, wherein the immune effector cell-binding strand is no more than 73 nucleotides in length.

54. The bispecific personalized aptamer of any one of claims 1-53, wherein the bispecific personalized aptamer comprises a combination of a cancer cell-binding strand selected from SEQ ID NOs: 43-62 or 107-115 and an immune effector cell-binding strand selected from SEQ ID NOs: 1-42, 88-106 or 116.

55. The bispecific personalized aptamer of any one of claims 1 to 54, wherein the aptamer comprises a chemical modification.

56. The bispecific personalized aptamer of claim 55, wherein the aptamer is chemically modified with poly-ethylene glycol (PEG).

57. The bispecific personalized aptamer of claim 56, wherein the PEG is attached to the 5′ end or the 3′ end of the aptamer.

58. The bispecific personalized aptamer of any one of claims 55 to 57, wherein the aptamer comprises a 5′ end cap.

59. The bispecific personalized aptamer of any one of claims 55 to 58, wherein the aptamer comprises a 3′ end cap.

60. The bispecific personalized aptamer of claim 59, wherein the 3′ end cap is an inverted thymidine.

61. The bispecific personalized aptamer of claim 59, wherein the 3′ end cap comprises biotin.

62. The bispecific personalized aptamer of any one of claims 55 to 61, wherein the aptamer comprises a 2′ sugar substitution.

63. The bispecific personalized aptamer of claims 62, wherein the 2′ sugar substitution is a 2′-fluoro, a 2′-amino, or a 2′-O-methyl substitution.

64. The bispecific personalized aptamer of any one of claims 55 to 63, wherein the aptamer comprises a locked nucleic acid (LNA), unlocked nucleic acid (UNA) and/or 2′deozy-2′fluoro-D-arabinonucleic acid (2′-F ANA) sugars in its backbone.

65. The bispecific personalized aptamer of any one of claims 55 to 64, wherein the aptamer comprises a methylphosphonate internucleotide bond and/or a phosphorothioate (PS) internucleotide bond.

66. The bispecific personalized aptamer of any one of claims 55 to 65, wherein the double-stranded CpG motif sequence comprises a partial PS modification.

67. The bispecific personalized aptamer of any one of claims 55 to 66, wherein 5 nucleotides from 5′ ends of the double-stranded CpG motif sequence are modified.

68. The bispecific personalized aptamer of any one of claims 55 to 67, wherein 5 nucleotides from both 5′ and 3′ ends of the double-stranded CpG motif sequence are modified.

69. The bispecific personalized aptamer of any one of claims 55 to 68, wherein the double-stranded CpG motif sequence comprises a complete PS modification.

70. The bispecific personalized aptamer of any one of claims 55 to 69, wherein the aptamer comprises a triazole internucleotide bond.

71. The bispecific personalized aptamer of any one of claims 55 to 70, wherein the aptamer is modified with a cholesterol or a dialkyl lipid.

72. The bispecific personalized aptamer of claim 71, wherein the cholesterol or diakyl lipid is linked to the 5′ end of the aptamer.

73. The bispecific personalized aptamer of any one of claims 55 to 72, wherein the aptamer comprises a modified base.

74. The bispecific personalized aptamer of any one of claims 1 to 73, wherein the aptamer is a DNA aptamer.

75. The bispecific personalized aptamer of claim 74, wherein the aptamer is a D-DNA aptamer.

76. The bispecific personalized aptamer of claim 75, wherein the aptamer is an R-DNA aptamer.

77. The bispecific personalized aptamer of any one of claims 1 to 73, wherein the aptamer is an RNA aptamer.

78. The bispecific personalized aptamer of claim 77, wherein the aptamer is a D-RNA aptamer.

79. The bispecific personalized aptamer of claim 77, wherein the aptamer is an R-RNA aptamer.

80. A pharmaceutical composition, comprising a bispecific personalized aptamer of any one of claims 1-79.

81. The pharmaceutical composition of claim 80, further comprising a pharmaceutically acceptable carrier.

82. The pharmaceutical composition of claim 80 or 81, wherein the pharmaceutical composition is formulated for parenteral administration.

83. The pharmaceutical composition of any one of claims 80 to 82, for use in treating cancer.

84. The pharmaceutical composition of claim 83, wherein the cancer is a solid tumor.

85. The pharmaceutical composition of claim 84, wherein the cancer is a breast cancer.

86. The pharmaceutical composition of claim 83, wherein the cancer is a carcinoma.

87. The pharmaceutical composition of claim 86, wherein the cancer is a colorectal carcinoma.

88. A method of treating cancer, the method comprising administering to a subject a bispecific personalized aptamer of any one of claims 1 to 87.

89. A method of treating cancer, the method comprising administering to a subject a pharmaceutical composition of any one of claims 80 to 88.

90. The method of claim 88 or 89, wherein the administration is parenteral administration.

91. The method of claim 90, wherein the administration is subcutaneous administration.

92. The method of claim 90 or 91, wherein the administration is an intratumoral injection.

93. The method of claim 90 or 91, wherein the administration is a peritumoral injection.

94. The method of any one of claims 88-93, wherein two or more doses are administered.

95. The method of any one of claims 88-94, wherein at least 10 to 12 doses are administered.

96. The method of any one of claims 88-95, wherein the administration to the subject of the two or more doses are separated by at least 1 day.

97. The method of any one of claims 88-96, wherein the cancer is a solid tumor.

98. The method of claim 97, wherein the solid tumor is accessible by intratumoral administration.

99. The method of claim 98, wherein the cancer is a breast cancer, head and neck squamous cell carcinoma, adenoid cystic carcinoma, bladder cancer, pancreatic cancer, hepatocellular carcinoma, melanoma, merkel cell carcinoma, or a colorectal carcinoma.

100. The method of any one of claims 88-97, wherein the cancer is a sarcoma.

101. The method of claim 100, wherein the cancer is a hematologic cancer.

102. The method of any one of claims 88-101, wherein the subject is a subject who has received chemotherapy.

103. The method of any one of claims 88-102, wherein the subject has had a tumor surgically removed.

104. The method of any one of claims 88-103, further comprising administering to the subject an additional cancer therapy.

105. The method of claim 104, wherein the additional cancer therapy comprises chemotherapy.

106. The method of claim 104, wherein the additional cancer therapy comprises radiation therapy.

107. The method of claim 104, wherein the additional cancer therapy comprises surgical removal of a tumor.

108. The method of claim 104, wherein the additional cancer therapy comprises administration of an immune checkpoint inhibitor to the subject.

109. The method of claim 108, wherein the immune checkpoint inhibitor is an anti-PD-1 antibody, an anti-PD-L1 antibody, an anti-PD-L2 antibody, or an anti-CTLA4 antibody.

110. A method of killing a cancer cell, the method comprising contacting the cancer cell with an aptamer of any one of claims 1 to 87.

111. The method of claim 110, wherein the cancer cell is killed by apoptosis, necrosis, immunological cell death, autophagy or necroptosis.

112. The method of claim 110 or 111, wherein the cancer cell is a solid tumor cell.

113. The method of claim 112, wherein the cancer cell is a breast cancer cell or a colorectal carcinoma cell.

114. The method of claim 110 or 111, wherein the cancer cell is a sarcoma cell.

115. The method of claim 110 or 111, wherein the cancer cell is a hematologic cell.

116. A method of making a bispecific personalized aptamer comprises: (1) synthesizing a cancer cell-binding strand; (2) synthesizing an immune effector cell-binding strand; (3) linking both strands to form the bispecific aptamer; optionally wherein the two strands are linked by hybridization, a covalent bond, or a PEG bridge.

117. The method of claim 116, wherein the cancer cell-binding strand is identified via a process comprising:
(a) contacting a cancer cells with a plurality of particles on which are immobilized a library of aptamer clusters (“aptamer cluster particles”), wherein at least a subset of the immobilized aptamer clusters bind to at least a subset of the cancer cell to form cell-aptamer cluster particle complexes;
(b) incubating the cell-aptamer cluster particle complexes for a period of time sufficient for at least some of the cancer cell in the cell-aptamer cluster particle complexes to undergo cell function;
(c) detecting the cell-aptamer cluster particle complexes undergoing the cell function;
(d) separating cell-aptamer cluster particle complexes comprising cancer cell undergoing the cell function detected in step (c) from other cell-aptamer cluster particle complexes;
(e) amplifying the aptamers in the separated cell-aptamer cluster particle complexes to generate a functionally enriched population of aptamers; and
(f) identifying the enriched population of aptamers via sequencing, thereby identifying the cancer cell-binding strand.

118. The method of claim 117, wherein steps (c) and (d) are performed using a flow cytometer.

119. The method of claim 117 or claim 118, further comprising separating the aptamer cluster particles from the target cells in the cell-aptamer cluster particle complexes separated in step (d).

120. The method of claim 119, further comprising the step of dissociating the aptamers from the particles in the separated aptamer cluster particles.

121. The method of any one of claims 117 to 120, further comprising a step (e′) after step (e) and before step (f): (i) forming aptamer cluster particles from the functionally enriched population of aptamers of step (e); and (ii) repeating steps (a) - (e) using the newly formed aptamer cluster particles to generate a further functionally enriched population of aptamers.

122. The method of claim 121, wherein step (e′) is repeated at least 2 times.

123. The method of claim 122, wherein step (e′) is repeated at least 3 times.

124. The method of claim 123, wherein step (e′) is repeated at least 4 times.

125. The method any one of claims 121-124, wherein step (e′) further comprises applying a restrictive condition in the successive rounds of enrichment.

126. The method of claim 125, wherein the restrictive condition is selected from: (i) reducing the total number of particles, (ii) reducing copy number of aptamers per particle, (iii) reducing the total number of target cells, (iv) reducing the incubation period, and (v) introducing errors to the aptamer sequences by amplifying the population of aptamers using error-prone polymerase.

127. The method of any of claims 121-126, wherein the further enriched population of aptamers of step (e′) has decreased sequence diversity compared to the library of aptamer clusters of step (a) by a factor of 2.

128. The method of any one of claims 121-127, wherein each round of step (e′) enriches the population of aptamers for aptamers that modulate the cellular function by a factor of at least 1.1.

129. The method of any one of claims 117-128, wherein the period of time is from about 10 minutes to about 5 days.

130. The method of any one of claims 117-129, wherein the period of time is from about 1.5 hours to about 72 hours.

131. The method of any one of claims 117-130, wherein the period of time is from about 1.5 hours to about 24 hours.

132. The method of any one of claims 117 to 131, wherein the cancer cell is contacted with a reporter of the cell function prior to, during, or after contacting the cancer cell with the aptamer cluster particles.

133. The method of any one of claims 117 to 131, wherein the cancer cell is contacted with the reporter of the cell function prior to, during, or after step (b).

134. The method of any one of claims 117 to 133, wherein the reporter of the cell function is a fluorescent dye.

135. The method of any one of claims 117-134, further comprising the step of isolating the cancer cell from a patient prior to step (a).

136. The method of claim 135, wherein the cancer cell is isolated from a tumor biopsy or resection.

137. The method of any one of claims 117-134, wherein the cell function is cell viability, cell death, or cell proliferation.

138. The method of any one of claims 116-137, wherein the synthesized cancer cell-binding strand and the synthesized immune effector cell-binding strand further comprise complementary 5′ sequences.

139. The method of claim 138, wherein the step (3) comprises hybridizing the synthesized cancer cell-binding strand and the synthesized immune effector cell-binding strand.

140. The method of claim 138, wherein the complementary 5′ sequence comprising a CpG-motif.

141. The method of any one of claims 116-140, wherein the complementary 5′ sequence comprises a nucleic acid sequence that is at least 80% identical to any one of SEQ ID NOs: 63-66.

142. The method of any one of claims 116-141, wherein the complementary 5′ sequence comprises a nucleic acid sequence that is at least 90% identical to any one of SEQ ID NOs: 63-66.

143. The method of any one of claims 116-142, wherein the complementary 5′ sequence comprises a nucleic acid sequence that is at least 95% identical to any one of SEQ ID NOs: 63-66.

144. The method of any one of claims 116-143, wherein the complementary 5′ sequence comprises a nucleic acid sequence that is at least 98% identical to any one of SEQ ID NOs: 63-66.

145. The method of any one of claims 116-144, wherein the complementary 5′ sequence comprises a nucleic acid sequence of any one of SEQ ID NOs: 63-66.

146. The method of any one of claims 116-145, wherein the cancer cell-binding strand comprises a nucleic acid sequence that is at least 80% identical to any one of SEQ ID NOs: 43-62 or 107-115.

147. The method of any one of claims 116-146, wherein the cancer cell-binding strand comprises a nucleic acid sequence that is at least 90% identical to any one of SEQ ID NOs: 43-62 or 107-115.

148. The method of any one of claims 116-147, wherein the cancer cell-binding strand comprises a nucleic acid sequence that is at least 95% identical to any one of SEQ ID NOs: 43-62 or 107-115.

149. The method of any one of claims 116-148, wherein the cancer cell-binding strand comprises a nucleic acid sequence that is at least 98% identical to any one of SEQ ID NOs: 43-62 or 107-115.

150. The method of any one of claims 116-149, wherein the cancer cell-binding strand comprises a nucleic acid sequence of any one of SEQ ID NOs: 43-62 or 107-115.

151. The method of any one of claims 116-150, wherein the immune effector cell-binding strand comprises a nucleic acid sequence that is at least 80% identical to any one of SEQ ID NOs: 1-42, 88-106 or 116.

152. The method of any one of claims 116-151, wherein the immune effector cell-binding strand comprises a nucleic acid sequence that is at least 90% identical to any one of SEQ ID NOs: 1-42, 88-106 or 116.

153. The method of any one of claims 116-152, wherein the immune effector cell-binding strand comprises a nucleic acid sequence that is at least 95% identical to any one of SEQ ID NOs: 1-42, 88-106 or 116.

154. The method of any one of claims 116-153, wherein the immune effector cell-binding strand comprises a nucleic acid sequence that is at least 98% identical to any one of SEQ ID NOs: 1-42, 88-106 or 116.

155. The method of any one of claims 116-154, wherein the immune effector cell-binding strand comprises a nucleic acid sequence of any one of SEQ ID NOs: 1-42, 88-106 or 116.

156. A method of treating cancer in a subject comprising administering to the subject a bispecific personalized aptamer made with the method of claims 116-155.


SEQUENCE LISTING (137 sequences)

SEQ ID NO: 1 - DNA, 50 bp, Artificial Sequence
  source: /note="Description of Artificial Sequence: Synthetic oligonucleotide"
tacgcgcaattcgccttgtcggtgatcttcctttgaacttgggcagtctg

SEQ ID NO: 2 - DNA, 50 bp, Artificial Sequence
  source: /note="Description of Artificial Sequence: Synthetic oligonucleotide"
tggcctggccgtgtcgtctgctttatagtcggtgatcccttgtgttaatt

SEQ ID NO: 3 - DNA, 50 bp, Artificial Sequence
  source: /note="Description of Artificial Sequence: Synthetic oligonucleotide"
gcatacctttcgtatgcctttttgacccgtatttttgccctacccttcgg

SEQ ID NO: 4 - DNA, 50 bp, Artificial Sequence
  source: /note="Description of Artificial Sequence: Synthetic oligonucleotide"
tttttcgctatccaacccttctttccagcctgccaatcagtcggtgatca

SEQ ID NO: 5 - DNA, 50 bp, Artificial Sequence
  source: /note="Description of Artificial Sequence: Synthetic oligonucleotide"
agggcagtcctgtatcttaacattctcctacatccgtaagtcggtgatcc

SEQ ID NO: 6 - DNA, 50 bp, Artificial Sequence
  source: /note="Description of Artificial Sequence: Synthetic oligonucleotide"
gggctaagagtctctattgtcggcagtcgtctaatatttcccgtccaatt

SEQ ID NO: 7 - DNA, 50 bp, Artificial Sequence
  source: /note="Description of Artificial Sequence: Synthetic oligonucleotide"
acttccggtgatttgatttcacttcctgggcagtcaatgtgattctctac

SEQ ID NO: 8 - DNA, 50 bp, Artificial Sequence
  source: /note="Description of Artificial Sequence: Synthetic oligonucleotide"
ggtcagtcgcctttgtcggtgatgtactcgcgcagtcgggttccccttaa

SEQ ID NO: 9 - DNA, 50 bp, Artificial Sequence
  source: /note="Description of Artificial Sequence: Synthetic oligonucleotide"
gggtctgttggtcctagggcagtcgtacttctaattcttgtcccgatgat

SEQ ID NO: 10 - DNA, 50 bp, Artificial Sequence
  source: /note="Description of Artificial Sequence: Synthetic oligonucleotide"
cttgtcggtgatctatagtcggtgatatattttgtcctatggtagtcgat

SEQ ID NO: 11 - DNA, 50 bp, Artificial Sequence
  source: /note="Description of Artificial Sequence: Synthetic oligonucleotide"
gggctcatgggcagtctttttactacctcctatttacgtatcccgctcct

SEQ ID NO: 12 - DNA, 50 bp, Artificial Sequence
  source: /note="Description of Artificial Sequence: Synthetic oligonucleotide"
cacccgcgcatttccccccagtcggtgattcttatatgtacctgttcctc

SEQ ID NO: 13 - DNA, 50 bp, Artificial Sequence
  source: /note="Description of Artificial Sequence: Synthetic oligonucleotide"
gggcacgtccattcgcgtttttgttccgtttctccctttttggattttgc

SEQ ID NO: 14 - DNA, 50 bp, Artificial Sequence
  source: /note="Description of Artificial Sequence: Synthetic oligonucleotide"
cagtcggtgtcactccagcggtcggttcactccacattctcccatctgtc

SEQ ID NO: 15 - DNA, 50 bp, Artificial Sequence
  source: /note="Description of Artificial Sequence: Synthetic oligonucleotide"
ggcagtcaccattctctttgggcagattgtctctcatccatatgtctcct

SEQ ID NO: 16 - DNA, 50 bp, Artificial Sequence
  source: /note="Description of Artificial Sequence: Synthetic oligonucleotide"
ctacctccttagtcggtgattcgatctatgggcctaactgccttctctgt

SEQ ID NO: 17 - DNA, 50 bp, Artificial Sequence
  source: /note="Description of Artificial Sequence: Synthetic oligonucleotide"
gggatgcggggccccgttctttttgtctctcattttgtcactttttttgt

SEQ ID NO: 18 - DNA, 50 bp, Artificial Sequence
  source: /note="Description of Artificial Sequence: Synthetic oligonucleotide"
ggtcagtcccttcggcatgtcgggattccctcttttcgcctcgtttcttt

SEQ ID NO: 19 - DNA, 50 bp, Artificial Sequence
  source: /note="Description of Artificial Sequence: Synthetic oligonucleotide"
ggctgtcgaactttctccctcccaccgcagtcggcccctcatcagtcgta

SEQ ID NO: 20 - DNA, 50 bp, Artificial Sequence; identical to SEQ ID NO: 7
  source: /note="Description of Artificial Sequence: Synthetic oligonucleotide"

SEQ ID NO: 21 - DNA, 50 bp, Artificial Sequence
  source: /note="Description of Artificial Sequence: Synthetic oligonucleotide"
acgtctgtcggtgacctgtaatagtttatgtcggtgatacagctttccct

SEQ ID NO: 22 - DNA, 50 bp, Artificial Sequence
  source: /note="Description of Artificial Sequence: Synthetic oligonucleotide"
ctgtcggtgatcatataacgcagtcggtgtagtttaatcccactccccta

SEQ ID NO: 23 - DNA, 50 bp, Artificial Sequence
  source: /note="Description of Artificial Sequence: Synthetic oligonucleotide"
ggccagtgtcccagtcgtgattgtaatattagattctttgtggcagtcgt

SEQ ID NO: 24 - DNA, 50 bp, Artificial Sequence
  source: /note="Description of Artificial Sequence: Synthetic oligonucleotide"
actcgtcggtgattttagacctttctcggtgatcaacacgtcatgctatt

SEQ ID NO: 25 - DNA, 50 bp, Artificial Sequence
  source: /note="Description of Artificial Sequence: Synthetic oligonucleotide"
gcctcgatatcctcaggagtcggtgtttcattcaatcgtcggtgataaat

SEQ ID NO: 26 - DNA, 50 bp, Artificial Sequence
  source: /note="Description of Artificial Sequence: Synthetic oligonucleotide"
ggtcagtccgtataccgccaatccgaaccgcagtcggtgtccgcttttac

SEQ ID NO: 27 - DNA, 50 bp, Artificial Sequence
  source: /note="Description of Artificial Sequence: Synthetic oligonucleotide"
tcgggttagatgtcggtcccactatatgtcggtgatctaatattgaactt

SEQ ID NO: 28 - DNA, 73 bp, Artificial Sequence
  source: /note="Description of Artificial Sequence: Synthetic oligonucleotide"
tcgtcgtcgcggttcgcgtccgtgcatacctttcgtatgcctttttgacccgtatttttgccctacccttcgg

SEQ ID NO: 29 - DNA, 73 bp, Artificial Sequence; identical to SEQ ID NO: 28
  source: /note="Description of Artificial Sequence: Synthetic oligonucleotide"

SEQ ID NO: 30 - DNA, 73 bp, Artificial Sequence; identical to SEQ ID NO: 28
  source: /note="Description of Artificial Sequence: Synthetic oligonucleotide"

SEQ ID NO: 31 - DNA, 73 bp, Artificial Sequence; identical to SEQ ID NO: 28
  source: /note="Description of Artificial Sequence: Synthetic oligonucleotide"

SEQ ID NO: 32 - DNA, 73 bp, Artificial Sequence
  source: /note="Description of Artificial Sequence: Synthetic oligonucleotide"
tcgtcgtcgcggttcgcgtccgtgggctaagagtctctattgtcggcagtcgtctaatatttcccgtccaatt

SEQ ID NO: 33 - DNA, 73 bp, Artificial Sequence
  source: /note="Description of Artificial Sequence: Synthetic oligonucleotide"
tcgtcgtcgcggttcgcgtccgtagggcagtcctgtatcttaacattctcctacatccgtaagtcggtgatcc

SEQ ID NO: 34 - DNA, 73 bp, Artificial Sequence
  source: /note="Description of Artificial Sequence: Synthetic oligonucleotide"
cttaatcagacattatacaaattgcatacctttcgtatgcctttttgacccgtatttttgccctacccttcgg

SEQ ID NO: 35 - DNA, 73 bp, Artificial Sequence
  source: /note="Description of Artificial Sequence: Synthetic oligonucleotide"
cttaatcagacattatacaaattgggctaagagtctctattgtcggcagtcgtctaatatttcccgtccaatt

SEQ ID NO: 36 - DNA, 73 bp, Artificial Sequence
  source: /note="Description of Artificial Sequence: Synthetic oligonucleotide"
cttaatcagacattatacaaattagggcagtcctgtatcttaacattctcctacatccgtaagtcggtgatcc

SEQ ID NO: 37 - DNA, 71 bp, Artificial Sequence
  source: /note="Description of Artificial Sequence: Synthetic oligonucleotide"
gaattaacaattataacgtttgcatacctttcgtatgcctttttgacccgtatttttgccctacccttcgg

SEQ ID NO: 38 - DNA, 71 bp, Artificial Sequence
  source: /note="Description of Artificial Sequence: Synthetic oligonucleotide"
gaattaacaattataacgtttagggcagtcctgtatcttaacattctcctacatccgtaagtcggtgatcc

SEQ ID NO: 39 - DNA, 69 bp, Artificial Sequence
  source: /note="Description of Artificial Sequence: Synthetic oligonucleotide"
gaattaacaattataacgtgggctaagagtctctattgtcggcagtcgtctaatatttcccgtccaatt

SEQ ID NO: 40 - DNA, 34 bp, Artificial Sequence
  source: /note="Description of Artificial Sequence: Synthetic oligonucleotide"
ccactgcgggggtctatacgtgaggaagaagtgg

SEQ ID NO: 41 - DNA, 57 bp, Artificial Sequence
  source: /note="Description of Artificial Sequence: Synthetic oligonucleotide"
tcgtcgtcggcgttcgcgtccgtccactgcgggggtctatacgtgaggaagaagtgg

SEQ ID NO: 42 - DNA, 53 bp, Artificial Sequence
  source: /note="Description of Artificial Sequence: Synthetic oligonucleotide"
gaattaacaattataacgtccactgcgggggtctatacgtgaggaagaagtgg

SEQ ID NO: 43 - DNA, 50 bp, Artificial Sequence
  source: /note="Description of Artificial Sequence: Synthetic oligonucleotide"
tccttgtcagcactttcagagcactttcccgtagaacttaagggacatgc

SEQ ID NO: 44 - DNA, 50 bp, Artificial Sequence
  source: /note="Description of Artificial Sequence: Synthetic oligonucleotide"
gattgatctattttccatatcgcgttgagtgtaaagccacgaagggttat

SEQ ID NO: 45 - DNA, 40 bp, Artificial Sequence
  source: /note="Description of Artificial Sequence: Synthetic oligonucleotide"
attggagttttccaatcagaaaggattcggtcagctgcac

SEQ ID NO: 46 - DNA, 40 bp, Artificial Sequence
  source: /note="Description of Artificial Sequence: Synthetic oligonucleotide"
tggaaacagctgcaacttttctgggacgtgaatgcctcgc

SEQ ID NO: 47 - DNA, 40 bp, Artificial Sequence
  source: /note="Description of Artificial Sequence: Synthetic oligonucleotide"
actcaaaaattaggcaggtgtaagtataactcgtgcctgc

SEQ ID NO: 48 - DNA, 73 bp, Artificial Sequence
  source: /note="Description of Artificial Sequence: Synthetic oligonucleotide"
cggacgcgaacgccgacgacgattccttgtcagcactttcagagcactttcccgtagaacttaagggacatgc

SEQ ID NO: 49 - DNA, 73 bp, Artificial Sequence
  source: /note="Description of Artificial Sequence: Synthetic oligonucleotide"
cggacgcgaaccgcgacgacgatgattgatctattttccatatcgcgttgagtgtaaagccacgaagggttat

SEQ ID NO: 50 - DNA, 73 bp, Artificial Sequence; identical to SEQ ID NO: 49
  source: /note="Description of Artificial Sequence: Synthetic oligonucleotide"

SEQ ID NO: 51 - DNA, 73 bp, Artificial Sequence; identical to SEQ ID NO: 49
  source: /note="Description of Artificial Sequence: Synthetic oligonucleotide"

SEQ ID NO: 52 - DNA, 73 bp, Artificial Sequence; identical to SEQ ID NO: 49
  source: /note="Description of Artificial Sequence: Synthetic oligonucleotide"

SEQ ID NO: 53 - DNA, 63 bp, Artificial Sequence
  source: /note="Description of Artificial Sequence: Synthetic oligonucleotide"
cggacgcgaaccgcgacgacgatattggagttttccaatcagaaaggattcggtcagctgcac

SEQ ID NO: 54 - DNA, 63 bp, Artificial Sequence
  source: /note="Description of Artificial Sequence: Synthetic oligonucleotide"
cggacgcgaaccgcgacgacgattggaaacagctgcaacttttctgggacgtgaatgcctcgc

SEQ ID NO: 55 - DNA, 63 bp, Artificial Sequence
  source: /note="Description of Artificial Sequence: Synthetic oligonucleotide"
cggacgcgaaccgcgacgacgatactcaaaaattaggcaggtgtaagtataactcgtgcctgc

SEQ ID NO: 56 - DNA, 73 bp, Artificial Sequence
  source: /note="Description of Artificial Sequence: Synthetic oligonucleotide"
atttgtataatgtctgattaagttccttgtcagcactttcagagcactttcccgtagaacttaagggacatgc

SEQ ID NO: 57 - DNA, 73 bp, Artificial Sequence
  source: /note="Description of Artificial Sequence: Synthetic oligonucleotide"
atttgtataatgtctgattaagtgattgatctattttccatatcgcgttgagtgtaaagccacgaagggttat

SEQ ID NO: 58 - DNA, 63 bp, Artificial Sequence
  source: /note="Description of Artificial Sequence: Synthetic oligonucleotide"
atttgtataatgtctgattaagtattggagttttccaatcagaaaggattcggtcagctgcac

SEQ ID NO: 59 - DNA, 63 bp, Artificial Sequence
  source: /note="Description of Artificial Sequence: Synthetic oligonucleotide"
atttgtataatgtctgattaagttggaaacagctgcaacttttctgggacgtgaatgcctcgc

SEQ ID NO: 60 - DNA, 63 bp, Artificial Sequence
  source: /note="Description of Artificial Sequence: Synthetic oligonucleotide"
atttgtataatgtctgattaagtactcaaaaattaggcaggtgtaagtataactcgtgcctgc

SEQ ID NO: 61 - DNA, 69 bp, Artificial Sequence
  source: /note="Description of Artificial Sequence: Synthetic oligonucleotide"
cgttataattgttaattcttccttgtcagcactttcagagcactttcccgtagaacttaagggacatgc

SEQ ID NO: 62 - DNA, 69 bp, Artificial Sequence
  source: /note="Description of Artificial Sequence: Synthetic oligonucleotide"
cgttataattgttaattctgattgatctattttccatatcgcgttgagtgtaaagccacgaagggttat

SEQ ID NO: 63 - DNA, 22 bp, Artificial Sequence
  source: /note="Description of Artificial Sequence: Synthetic oligonucleotide"
tcgtcgtcgcggttcgcgtccg

SEQ ID NO: 64 - DNA, 22 bp, Artificial Sequence
  source: /note="Description of Artificial Sequence: Synthetic oligonucleotide"
cggacgcgaacgccgacgacga

SEQ ID NO: 65 - DNA, 22 bp, Artificial Sequence
  source: /note="Description of Artificial Sequence: Synthetic oligonucleotide"
cgtcgtcggtcgtcgtcgctcg

SEQ ID NO: 66 - DNA, 22 bp, Artificial Sequence
  source: /note="Description of Artificial Sequence: Synthetic oligonucleotide"
cgagcgacgacgaccgacgacg

SEQ ID NO: 67 - DNA, 22 bp, Artificial Sequence
  source: /note="Description of Artificial Sequence: Synthetic oligonucleotide"
cttaatcagacattatacaaat

SEQ ID NO: 68 - DNA, 22 bp, Artificial Sequence
  source: /note="Description of Artificial Sequence: Synthetic oligonucleotide"
atttgtataatgtctgattaag

SEQ ID NO: 69 - DNA, 18 bp, Artificial Sequence
  source: /note="Description of Artificial Sequence: Synthetic oligonucleotide"
gaattaacaattataacg

SEQ ID NO: 70 - DNA, 18 bp, Artificial Sequence
  source: /note="Description of Artificial Sequence: Synthetic oligonucleotide"
cgttataattgttaattc

SEQ ID NO: 71 - DNA, 89 bp, Artificial Sequence
  source: /note="Description of Artificial Sequence: Synthetic oligonucleotide"
  modified_base (21)..(69): a, c, t, g, unknown or other
tatccgtctgctctcgctatnnnnnnnnnnnnnnnnnnnnnnnnnnnnnnnnnnnnnnnnnnnnnnnnnacgcacctaatgtcctactg

SEQ ID NO: 72 - DNA, 20 bp, Artificial Sequence
  source: /note="Description of Artificial Sequence: Synthetic primer"
tatccgtctgctctcgctat

SEQ ID NO: 73 - DNA, 20 bp, Artificial Sequence
  source: /note="Description of Artificial Sequence: Synthetic primer"
cagtaggacattaggtgcgt

SEQ ID NO: 74 - DNA, 20 bp, Artificial Sequence
  source: /note="Description of Artificial Sequence: Synthetic oligonucleotide"
atagcgagagcagacggata

SEQ ID NO: 75 - DNA, 20 bp, Artificial Sequence
  source: /note="Description of Artificial Sequence: Synthetic primer"
tatccgtctgctctcgctat

SEQ ID NO: 76 - DNA, 69 bp, Artificial Sequence
  source: /note="Description of Artificial Sequence: Synthetic oligonucleotide"
gaattaacaattataacgttttttttttttttttttttttttttttttttttttttttttttttttttt

SEQ ID NO: 77 - DNA, 69 bp, Artificial Sequence
  source: /note="Description of Artificial Sequence: Synthetic oligonucleotide"
cgttataattgttaattcttttttttttttttttttttttttttttttttttttttttttttttttttt

SEQ ID NO: 78 - DNA, 73 bp, Artificial Sequence
  source: /note="Description of Artificial Sequence: Synthetic oligonucleotide"
cttaatcagacattatacaaatttttttttttttttttttttttttttttttttttttttttttttttttttt

SEQ ID NO: 79 - DNA, 73 bp, Artificial Sequence
  source: /note="Description of Artificial Sequence: Synthetic oligonucleotide"
atttgtataatgtctgattaagttttttttttttttttttttttttttttttttttttttttttttttttttt

SEQ ID NO: 80 - DNA, 73 bp, Artificial Sequence
  source: /note="Description of Artificial Sequence: Synthetic oligonucleotide"
tcgtcgtcgcggttcgcgtccgttttttttttttttttttttttttttttttttttttttttttttttttttt

SEQ ID NO: 81 - DNA, 73 bp, Artificial Sequence
  source: /note="Description of Artificial Sequence: Synthetic oligonucleotide"
cggacgcgaacgccgacgacgattttttttttttttttttttttttttttttttttttttttttttttttttt

SEQ ID NO: 82 - DNA, 50 bp, Artificial Sequence
  source: /note="Description of Artificial Sequence: Synthetic oligonucleotide"
tttttttttttttttttttttttttttttttttttttttttttttttttt

SEQ ID NO: 83 - DNA, 54 bp, Artificial Sequence
  source: /note="Description of Artificial Sequence: Synthetic oligonucleotide"
ccgcgtccggacacctaatttggttcaagagccgcccgtaatttcaggttctcc

SEQ ID NO: 84 - DNA, 50 bp, Artificial Sequence; identical to SEQ ID NO: 3
  source: /note="Description of Artificial Sequence: Synthetic oligonucleotide"

SEQ ID NO: 85 - DNA, 50 bp, Artificial Sequence
  source: /note="Description of Artificial Sequence: Synthetic oligonucleotide"
gttcttataatcgcctctgcgctatgttcttgctcgccttccatatcgct

SEQ ID NO: 86 - DNA, 50 bp, Artificial Sequence; identical to SEQ ID NO: 85
  source: /note="Description of Artificial Sequence: Synthetic oligonucleotide"

SEQ ID NO: 87 - DNA, 50 bp, Artificial Sequence
  source: /note="Description of Artificial Sequence: Synthetic oligonucleotide"
tctttcgttagcgcttctctcttgcgattccgaccgcatattcacgtctt

SEQ ID NO: 88 - DNA, 40 bp, Artificial Sequence
  source: /note="Description of Artificial Sequence: Synthetic oligonucleotide"
atcgtataagggctgcttaggattgcgataatacggtcaa

SEQ ID NO: 89 - DNA, 40 bp, Artificial Sequence
  source: /note="Description of Artificial Sequence: Synthetic oligonucleotide"
catttcatagggctgcttaggattgcgaaggtaatgccag

SEQ ID NO: 90 - DNA, 40 bp, Artificial Sequence
  source: /note="Description of Artificial Sequence: Synthetic oligonucleotide"
cccttaccccttttaggtctgcttaggattgcgaaaaaag

SEQ ID NO: 91 - DNA, 40 bp, Artificial Sequence
  source: /note="Description of Artificial Sequence: Synthetic oligonucleotide"
ttgtaaggactgcttaggattgcgaaaacaatattcgtat

SEQ ID NO: 92 - DNA, 31 bp, Artificial Sequence
  source: /note="Description of Artificial Sequence: Synthetic oligonucleotide"
cttttaggtctgcttaggattgcgaaaaaag

SEQ ID NO: 93 - DNA, 40 bp, Artificial Sequence
  source: /note="Description of Artificial Sequence: Synthetic oligonucleotide"
tccatgggtctgctctaggattgcgttcatggtctccccg

SEQ ID NO: 94 - DNA, 40 bp, Artificial Sequence
  source: /note="Description of Artificial Sequence: Synthetic oligonucleotide"
aattacaaccttggattgcaaagggctgctgtgttgttta

SEQ ID NO: 95 - DNA, 40 bp, Artificial Sequence
  source: /note="Description of Artificial Sequence: Synthetic oligonucleotide"
atcggagctgttccttgataccgattcaaaaagttcgtac

SEQ ID NO: 96 - DNA, 40 bp, Artificial Sequence
  source: /note="Description of Artificial Sequence: Synthetic oligonucleotide"
aatttgtagggactgctcaggattgcggatacaaattaat

SEQ ID NO: 97 - DNA, 40 bp, Artificial Sequence
  source: /note="Description of Artificial Sequence: Synthetic oligonucleotide"
agacattggggactgctcgggattgcgaatctatgtctcc

SEQ ID NO: 98 - DNA, 40 bp, Artificial Sequence
  source: /note="Description of Artificial Sequence: Synthetic oligonucleotide"
cccttttttaactaggtctgcttaggattgcgaatgttaa

SEQ ID NO: 99 - DNA, 40 bp, Artificial Sequence
  source: /note="Description of Artificial Sequence: Synthetic oligonucleotide"
acctcaaaagcgcgggctgctcaaaggattgcgtagcttt

SEQ ID NO: 100 - DNA, 40 bp, Artificial Sequence
  source: /note="Description of Artificial Sequence: Synthetic oligonucleotide"
gggggttaagggctgcttaggattgcgataatacggtcaa

SEQ ID NO: 101 - DNA, 40 bp, Artificial Sequence
  source: /note="Description of Artificial Sequence: Synthetic oligonucleotide"
aacatataactgctcaataatatagataaaatactcacaa

SEQ ID NO: 102 - DNA, 40 bp, Artificial Sequence
  source: /note="Description of Artificial Sequence: Synthetic oligonucleotide"
ctctacctgactgtaacctctcgctcccccccattcgcgc

SEQ ID NO: 103 - DNA, 40 bp, Artificial Sequence
  source: /note="Description of Artificial Sequence: Synthetic oligonucleotide"
ttgtccctctacgccgccctttactaccactcctgcgatt

SEQ ID NO: 104 - DNA, 40 bp, Artificial Sequence
  source: /note="Description of Artificial Sequence: Synthetic oligonucleotide"
tccagcacaccgaccgcccctctacattaccccctggact

SEQ ID NO: 105 - DNA, 40 bp, Artificial Sequence
  source: /note="Description of Artificial Sequence: Synthetic oligonucleotide"
cccctccattcccccgcctcgtccaccctactccttagtc

SEQ ID NO: 106 - DNA, 40 bp, Artificial Sequence
  source: /note="Description of Artificial Sequence: Synthetic oligonucleotide"
catcgacgcccacacaccacttcccgttcccctgcatcat

SEQ ID NO: 107 - DNA, 40 bp, Artificial Sequence
  source: /note="Description of Artificial Sequence: Synthetic oligonucleotide"
gcaggcggaaaatgtcagggcacgttggtcacgtattttt

SEQ ID NO: 108 - DNA, 40 bp, Artificial Sequence
  source: /note="Description of Artificial Sequence: Synthetic oligonucleotide"
agcaatcatatggctgtgctcatttaataagcaagctggg

SEQ ID NO: 109 - DNA, 63 bp, Artificial Sequence
  source: /note="Description of Artificial Sequence: Synthetic oligonucleotide"
cggacgcgaaccgcgacgacgatgcaggcggaaaatgtcagggcacgttggtcacgtattttt

SEQ ID NO: 110 - DNA, 63 bp, Artificial Sequence
  source: /note="Description of Artificial Sequence: Synthetic oligonucleotide"
cggacgcgaaccgcgacgacgatagcaatcatatggctgtgctcatttaataagcaagctggg

SEQ ID NO: 111 - DNA, 63 bp, Artificial Sequence
  source: /note="Description of Artificial Sequence: Synthetic oligonucleotide"
cggacgcgaaccgcgacgacgataaactctatcgtccagagagaatgtctgcctactgatttg

SEQ ID NO: 112 - DNA, 40 bp, Artificial Sequence
  source: /note="Description of Artificial Sequence: Synthetic oligonucleotide"
gtgttagtgatgcgagctccttaccattagatagaggctg

SEQ ID NO: 113 - DNA, 40 bp, Artificial Sequence
  source: /note="Description of Artificial Sequence: Synthetic oligonucleotide"
gctgcgtcctccattagcgctgagacttacattcctatac

SEQ ID NO: 114 - DNA, 40 bp, Artificial Sequence
  source: /note="Description of Artificial Sequence: Synthetic oligonucleotide"
tccaagcataggacgataccttgcatttccttttcagatc

SEQ ID NO: 115 - DNA, 40 bp, Artificial Sequence
  source: /note="Description of Artificial Sequence: Synthetic oligonucleotide"
ggtatcttttcttcgtccattactatcggtgttcgaactc

SEQ ID NO: 116 - DNA, 63 bp, Artificial Sequence
  source: /note="Description of Artificial Sequence: Synthetic oligonucleotide"
tcgtcgtcgcggttcgcgtccgtatcgtataagggctgcttaggattgcgataatacggtcaa

SEQ ID NO: 117 - DNA, 80 bp, Artificial Sequence
  source: /note="Description of Artificial Sequence: Synthetic oligonucleotide"
  modified_base (21)..(60): a, c, t, g, unknown or other
tcactatcggtccagacgtannnnnnnnnnnnnnnnnnnnnnnnnnnnnnnnnnnnnnnntattgcgccgaggttcttac

SEQ ID NO: 118 - DNA, 20 bp, Artificial Sequence
  source: /note="Description of Artificial Sequence: Synthetic primer"
tcactatcggtccagacgta

SEQ ID NO: 119 - DNA, 20 bp, Artificial Sequence
  source: /note="Description of Artificial Sequence: Synthetic primer"
tcactatcggtccagacgta

SEQ ID NO: 120 - DNA, 20 bp, Artificial Sequence
  source: /note="Description of Artificial Sequence: Synthetic primer"
gtaagaacctcggcgcaata

SEQ ID NO: 121 - DNA, 20 bp, Artificial Sequence
  source: /note="Description of Artificial Sequence: Synthetic oligonucleotide"
tacgtctggaccgatagtga

SEQ ID NO: 122 - DNA, 25 bp, Artificial Sequence
  source: /note="Description of Artificial Sequence: Synthetic oligonucleotide"
agggctgcttaggattgcgataata

SEQ ID NO: 123 - DNA, 25 bp, Artificial Sequence
  source: /note="Description of Artificial Sequence: Synthetic oligonucleotide"
agggctgcttaggattgcgaaggta

SEQ ID NO: 124 - DNA, 26 bp, Artificial Sequence
  source: /note="Description of Artificial Sequence: Synthetic oligonucleotide"
gggtctgctctaggattgcgttcatg

SEQ ID NO: 125 - DNA, 26 bp, Artificial Sequence
  source: /note="Description of Artificial Sequence: Synthetic oligonucleotide"
ggagctgttccttgataccgattcaa

SEQ ID NO: 126 - DNA, 25 bp, Artificial Sequence
  source: /note="Description of Artificial Sequence: Synthetic oligonucleotide"
gggactgctcaggattgcggataca

SEQ ID NO: 127 - DNA, 25 bp, Artificial Sequence
  source: /note="Description of Artificial Sequence: Synthetic oligonucleotide"
aggtctgcttaggattgcgaaaaaa

SEQ ID NO: 128 - DNA, 25 bp, Artificial Sequence
  source: /note="Description of Artificial Sequence: Synthetic oligonucleotide"
gggactgctcgggattgcgaatcta

SEQ ID NO: 129 - DNA, 25 bp, Artificial Sequence
  source: /note="Description of Artificial Sequence: Synthetic oligonucleotide"
aggtctgcttaggattgcgaatgtt

SEQ ID NO: 130 - DNA, 25 bp, Artificial Sequence
  source: /note="Description of Artificial Sequence: Synthetic oligonucleotide"
aggactgcttaggattgcgaaaaca

SEQ ID NO: 131 - DNA, 27 bp, Artificial Sequence
  source: /note="Description of Artificial Sequence: Synthetic oligonucleotide"
cgggctgctcaaaggattgcgtagctt

SEQ ID NO: 132 - DNA, 26 bp, Artificial Sequence
  source: /note="Description of Artificial Sequence: Synthetic oligonucleotide"
ataactgctcaataatatagataaaa

SEQ ID NO: 133 - DNA, 27 bp, Artificial Sequence
  source: /note="Description of Artificial Sequence: Synthetic oligonucleotide"
taagggctgcttaggattgcgataata

SEQ ID NO: 134 - DNA, 27 bp, Artificial Sequence
  source: /note="Description of Artificial Sequence: Synthetic oligonucleotide"
atagggctgcttaggattgcgaaggta

SEQ ID NO: 135 - DNA, 27 bp, Artificial Sequence
  source: /note="Description of Artificial Sequence: Synthetic oligonucleotide"
ttaggtctgcttaggattgcgaaaaaa

SEQ ID NO: 136 - DNA, 27 bp, Artificial Sequence
  source: /note="Description of Artificial Sequence: Synthetic oligonucleotide"
taaggactgcttaggattgcgaaaaca

SEQ ID NO: 137 - DNA, 27 bp, Artificial Sequence
  source: /note="Description of Artificial Sequence: Synthetic oligonucleotide"
tggggactgctcgggattgcgaatcta